(19) United States
(12) Patent Application Publication
(10) Pub. No.: US 2025/0369024 A1
(43) Pub. Date: Dec. 4, 2025

(54) Acyloin Condensation Reactions, Enzymes, and Products Thereof

(71) Applicant: Mojia Biotech Pte. Ltd., Singapore (SG)

(72) Inventors: Ramon GONZALEZ, Tampa, FL (US); Seung Hwan LEE, Tampa, FL (US); Fayin ZHU, Tampa, FL (US)

(21) Appl. No.: 19/241,255

(22) Filed: Jun. 17, 2025

Related U.S. Application Data
(63) Continuation of application No. PCT/IB2024/000021, filed on Jan. 12, 2024.
(60) Provisional application No. 63/479,606, filed on Jan. 12, 2023.

(51) Int. Cl. C12P 7/62; C12N 9/10; C12N 9/88; C12P 7/625

(52) U.S. Cl. CPC C12P 7/62; C12N 9/1025; C12N 9/88; C12P 7/625; C12Y 401/01008

(57) ABSTRACT

The invention includes novel routes of synthesis that involve the use of an acyloin condensation reaction between a ketone and formyl-CoA to form a corresponding branched 2-hydroxyacyl-CoAs and forming branched chain products therefrom. Described are genetically modified microorganisms that produce a branched product from a ketone and formyl-CoA and methods of producing a branched product, including in vivo and in vitro methods.

### RELATED APPLICATIONS

This application is a continuation of International Application No. PCT/IB2024/000021, which designated the United States and was filed on Jan. 12, 2024, published in English, which claims the benefit of U.S. Provisional Application No. 63/479,606, filed on Jan. 12, 2023. The entire teachings of the above applications are incorporated herein by reference.

### SEQUENCE LISTING

The sequence listing submitted via EFS, in compliance with 37 CFR § 1.52(e)(5), is incorporated herein by reference. The sequence listing XML file submitted via EFS contains the file “43673006 US1 REVISED Seq List.xml”, created on Jun. 13, 2025, which is 4,479 bytes in size.

### BACKGROUND OF THE INVENTION

Naturally occurring, canonical metabolism relies mainly on the use of two or three carbon metabolites that serve as the building blocks for diverse biological chemistry. While these pathways have been exploited for the production of numerous chemical products, these approaches face limitations particularly in relation to the use of one-carbon substrates for chemical production. Existing approaches, for example, typically require first the production of two or three carbon metabolites from one-carbon substrates. Thus, there exists an opportunity to develop more direct routes to produce compounds using one carbon substrates.

The use of the enzyme 2-hydroxyacyl-CoA lyase (HACL) to catalyze a one-carbon elongation reaction has recently been reported. Specifically, the condensation of formyl-CoA with certain aldehydes and ketones of varying chain lengths to produce 2-hydroxyacyl-CoAs has been described in WO2016/069929A1, U.S. Pat. App. Pub. No. 20190100741A1, and WO2020/247430. WO2023287814 describes methods preparing the aldehydes and ketones that are condensed with formyl-CoA from carboxylic acid intermediates.

While these one-carbon elongation reactions enables an innovative platform for generating products from one-carbon substrates, it would be advantageous to provide methods for preparing additional products.

### SUMMARY OF THE INVENTION

The present invention encompasses novel routes of synthesis that involve the use of an acyloin condensation reaction between a ketone and formyl-CoA to form a corresponding branched 2-hydroxyacyl-CoAs and forming branched chain products (also referred to here as branched chain products) therefrom.

The invention encompasses genetically modified microorganisms that produce a product (e.g., a branched chain product) from a ketone and formyl-CoA, methods of producing a product (e.g., a branched chain product) from a ketone and formyl-CoA comprising growing a genetically modified microorganism described herein, and methods of producing a product (e.g., a branched chain product) from a ketone and formyl-CoA, including in vivo and in vitro (cell free) methods.

In certain aspects, the invention is a genetically modified microorganism that produces a branched product from a ketone and formyl-CoA, the microorganism comprising:
a. at least one heterologous DNA molecule encoding an enzyme that catalyzes the condensation of the ketone with the formyl-CoA to produce a branched 2-hydroxyacyl-CoA that is one carbon longer than the ketone; in certain aspect, the enzyme is a HACS; and
b. one or more heterologous DNA molecules encoding one or more enzymes that catalyzes the conversion the 2-hydroxyacyl-CoA to the product.
   In further aspects, the branched product is selected from the group consisting of a 2-hydroxyacid, an ab-unsaturated acid, a 1,2-diol (e.g. 1,2-Cₙ₊₁ diol wherein the ketone is a Cₙ-ketone and the 2-hydroxy-acylCoA is a 2-hydroxy-Cₙ₊₁-acylCoA), an alcohol (e.g., a Cₙ₊₁-alchohol wherein the ketone is a Cₙ-ketone), and a 3-hydroxyacid.

The invention also includes a method for producing a branched product from a ketone and formyl-CoA, the method comprising growing the microorganism described herein in the presence of a ketone or a carboxylic acid precursor thereof and in the presence of the formyl-CoA or a 1-carbon precursor thereof. When the microorganism is cultured in the presence of the carboxylic acid precursor (or wherein the culture comprises the carboxylic acid precursor), the microorganism expresses one more enzymes that catalyzes the conversion of the carboxylic acid precursor to the ketone; and when the microorganism is cultured in the presence of the 1-carbon precursor (or wherein the culture comprises the 1-carbon precursor), the microorganism expresses one or more enzymes that catalyze the conversion of the 1-carbon precursor to the formyl-CoA. In certain aspects, the branched product is selected from the group consisting of a 2-hydroxyacid, an ab-unsaturated acid, a 1,2-diol, an alcohol, and a 3-hydroxyacid.

In additional aspects, the invention is a method for producing a branched product from a ketone and formyl-CoA, the method comprising the steps of:
a. growing a genetically modified microorganism in a culture comprising a ketone or a carboxylic acid precursor thereof and comprising formyl-CoA or a 1-carbon precursor thereof;
   wherein the microorganism expresses an enzyme that catalyzes the condensation of the ketone with the formyl-CoA to produce a branched 2-hydroxyacyl-CoA that is one carbon longer than the ketone; and
   further wherein the microorganism expresses one or more enzymes that convert the 2-hydroxyacyl-CoA to the product;
b. condensing the ketone with the formyl-CoA to produce the 2-hydroxyacyl-CoA; and
c. converting the 2-hydroxyacyl-CoA to the product;
wherein when the culture comprises the carboxylic acid precursor, the microorganism expresses one more enzymes that catalyze the conversion of the carboxylic acid precursor to the ketone; and
wherein when the culture comprises the 1-carbon precursor, the microorganism expresses one or more enzymes that catalyze the conversion of the 1-carbon precursor to the formyl-CoA; and
wherein the product is selected from the group consisting of product is selected from the group consisting of a branched 2-hydroxyacid, an ab-unsaturated acid, a 1,2-diol, an alcohol, and a 3-hydroxyacid.

In further aspects, the invention is a method of producing a branched 2-hydroxy acid product from a ketone and formyl-CoA, comprising the steps of:
a. culturing a microorganism that is engineered to express one or more enzymes that catalyze the condensation of a ketone with a formyl-CoA to produce a branched 2-hydroxyacyl-CoA that is one carbon longer than the ketone; and
b. hydrolysis of the 2-hydroxyacyl-CoA to the branched 2-hydroxyacid product,
   wherein the microorganism is cultured in the presence of the ketone or a carboxylic acid precursor thereof and in the presence of the formyl-CoA or a 1-carbon precursor thereof; and
   wherein when the microorganism is cultured in the presence of the carboxylic acid precursor, the microorganism expresses one more enzymes that catalyze the conversion of the carboxylic acid precursor to the ketone; and wherein when the microorganism is cultured in the presence of the 1-carbon precursor, the microorganism expresses one or more enzymes that catalyze the conversion the 1-carbon precursor to the formyl-CoA.

In yet additional aspects, the invention is a method of producing a branched product from a ketone and formyl-CoA, comprising the steps of:
a. condensing a ketone with a formyl-CoA to produce a branched 2-hydroxyacyl-CoA that is one carbon longer than the ketone in the presence of an enzyme that catalyzes the condensation of the ketone with the formyl-CoA; and
b. converting the 2-hydroxyacyl-CoA to the product; optionally in the presence of one or more enzymes that catalyze the conversion, wherein the product is selected from the group consisting of a 2-hydroxyacid, an ab-unsaturated acid, a 1,2-diol, an alcohol, and a 3-hydroxyacid. In certain aspects, the method is a cell-free method. The ketone or carboxylic acid precursor thereof, formyl-CoA and precursor thereof, and enzymes can, for example, be present in a reaction mixture and the product can be isolated from the reaction mixture. In yet further aspects, step a is carried out by a genetically modified microorganism that expresses the enzyme that catalyzes the condensation of the ketone with the formyl-CoA. In additional aspects, step b is carried out by culturing a genetically modified microorganism that expresses the one or more enzymes that catalyze the conversion of the 2-hydroxacyl-CoA to the product. In yet further aspects, step a and/or step b are carried out in the presence of purified enzymes in a cell-free system. In certain embodiments, the product is a 2-hydroxy acid and the branched 2-hydroxyacyl-CoA is converted to the branched 2-hydroxy acid product by hydrolysis.

In the methods and microorganisms described herein, the condensation of the ketone with the formyl-CoA can be catalyzed by a 2-hydroxy-acyl-CoA-synthase (HACS). In certain aspects, the HACS is selected from the group consisting of 2-hydroxyacyl-CoA lyase (HACL), oxalyl-CoA decarboxylase and a benzaldehyde lyase. In certain aspects, the HACL is a mammalian HACL. In yet other aspects, the HACL is a prokaryotic HACL. In certain aspects, the HACL is Homo sapiens hacl1 (Q9UJ83), Rattus norvegicus hacl1 (Q8CHM7), Dictyostelium discoideum hacl1 (Q54DA9), Mus musculus hacl1 (Q9QXE0). In yet additional examples, the HACL is Rhodospirillales bacterium URHD0017 HACL (RuHACL). In yet further aspects, the HACS is selected from those in Table A below.

TABLE A

| | | Abbre- |
|---|---|---|
| Accession Nos. | Protein name | viation |
| GenBank Acc. No. | Actinomycetospora chiangmaiensis | AcHACS |
| WP_018331913.1 | DSM 45062 2-hydroxyacyl-CoA | |
| | lyase | |
| GenBank Acc. No. | Alphaproteobacteria bacterium | ApbHACS |
| HAK63664.1 | Oxalyl-CoA decarboxylase | |
| GenBank Acc. No. | hypothetical protein | AtsHACS |
| XP_012756082.1 | SAMD00019534_041230 | |
| | [Acytostelium subglobosum LB1] | |
| GenBank Acc. No. | MAG TPA: oxalyl-CoA | ApbHACS |
| HAK63664.1 | decarboxylase [Alphaproteobacteria | |
| | bacterium] | |
| GenBank Acc. No. | MAG: hypothetical protein | BtbHACS |
| OGA51379.1 | A3F74_17750 [Betaproteobacteria | |
| | bacterium | |
| | RIFCSPLOWO2_12_FULL_62_58] | |
| GenBank Acc. No. | MAG: oxalyl-CoA decarboxylase | DhcHACS |
| PWB41796.1 | [Dehalococcoidia bacterium] | |
| GenBank Acc. No. | 2-hydroxyacyl-CoA lyase 1 | OxtHACS |
| EJY87672.1 | (macronuclear) [Oxytricha trifallax] | |
| GenBank Acc. No. | thiamine pyrophosphate-binding | RhsHACS |
| WP_095860310.1 | protein [Rhodococcus sp. ACS1] | |
| GenBank Acc. No. | MAG: thiamine pyrophosphate- | RcbHACS |
| MBL8483477.1 | binding protein [Rhodocyclaceae | |
| | bacterium] | |
| GenBank Acc. No. | thiamine pyrophosphate-binding | AspHACS |
| WP_169186431.1 | protein [Actinomycetospora sp. | |
| | TBRC 11914] | |
| GenBank Acc. No. | MAG: oxalyl-CoA decarboxylase | CfbHACS |
| MBI2761137.1 | [Chloroflexi bacterium] | |
| GenBank Acc. No. | 2-hydroxyphytanoyl-CoA lyase | CoHACS |
| XP_004342722.2 | [Capsaspora owczarzaki ATCC | |
| | 30864] | |
| GenBank Acc. No. | oxalyl-CoA decarboxylase | CfhHACS |
| PKN81274.1 | [Chloroflexi bacterium HGW- | |
| | Chloroflexi-9] | |
| GenBank Acc. No. | acetolactate synthase | PspHACS |
| OJY48151.1 | [Pseudonocardia sp. 73-21] | |
| GenBank Acc. No. | thiamine pyrophosphate-binding | PdsHACS |
| WP_130292058.1 | protein [Pseudonocardia sediminis] | |
| Nature Chem | Engineered 2-hydroxyphytanoyl- | RuHACS/ |
| Biol., 2019, 15, | CoA lyase [Rhodospirillales | RuHACL |
| 900-906; SEQ ID | bacterium URHD0017] | |
| NO: 1 | | |
| ACS Catal. 2021, | Engineered oxalyl-CoA | MeOXC4 |
| 11, 9, 5396-5404; | decarboxylase [Methylorubrum | |
| SEQ ID NO: 2 | extorquens] | |

In a further aspect, the HACS is Alphaproteobacteria bacterium oxalyl-CoA decarboxylase (ApbHACS/BsmHACS) (UniProt accession: A0A3COTX30), CfhHACS, RcbHACS, PspHACS, Chacs, CfhHACS and DhcHACS. In yet further aspects, the HACS is ApbHACS, DhcHACS, RcbHACS, PdsHACS and PspHACS.

In certain aspects, the ketone has the Formula (I):

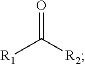

wherein:
R₁ and R₂ are each independently selected from the group consisting of C₁-C₁₂ alkyl, C₂-C₁₂ alkenyl, C₃-C₁₅ cycloalkenyl, aryl, heteroaryl, heterocyclyl, C(O)OR_{b}, C(O)R_{b}, SR_{b}, and N(R_{b})₂; wherein the C₁-C₁₂ alkyl, the C₂-C₁₂ alkenyl, the C₃-C₁₅ cycloalkenyl, the aryl, the heteroaryl, and the heterocyclyl are each optionally substituted; in certain aspects, the C₁-C₁₂ alkyl, the C₂-C₁₂ alkenyl, the C₃-C₁₅ cycloalkenyl, the aryl, the heteroaryl, and the heterocyclyl are each optionally substituted by one or more Rₐ;
alternatively, R₁ and R₂ are taken together to form a C₃ to C₇ cycloalkyl, a C₃ to C₇ cycloalkenyl, or a 3- to 7-membered heterocyclyl, wherein the cycloalkyl, cycloalkenyl, and heterocyclyl are each optionally substituted; in certain aspects, the cycloalkyl, cycloalkenyl, and heterocyclyl is optionally substituted by one or more Rₐ;
each Rₐ is independently selected from the group consisting of hydrogen, optionally substituted C₁-C₁₂ alkyl, optionally substituted C₂-C₁₂ alkenyl, optionally substituted C₂-C₁₂ alkynyl, optionally substituted C₃-C₁₅ cycloalkyl, optionally substituted C₃-C₁₅ cycloalkenyl, optionally substituted aryl, optionally substituted heteroaryl, optionally substituted heterocyclyl, C(O)R_{b}, C(O)OR_{b}, OR_{b}, N(R_{b})₂, halo, and oxo; and
each R_{b} is independently hydrogen or a C₁-C₆ alkyl.
   In certain aspects, when one of R₁ and R₂ is C(O)OR_{b} or C(O)R_{b}, the other of R₁ and R₂ is not C(O)OR_{b} or C(O)R_{b}.

### BRIEF DESCRIPTION OF DRAWINGS

FIG. 1 is a schematic showing the production of branched-chain compounds via formyl-CoA based elongation of ketones.

FIGS. 2A and 2B demonstrate condensation of ketones (a methyl ketone is shown) as substrates for condensation with formyl-CoA using purified enzymes. FIG. 2A shows a pathway for condensation of methyl ketones and formyl-CoA (produced from formate) to 2-hydroxy-2-methyl acid. FIG. 2B shows GC-MS results of in vitro assays with the shown methyl ketones (acetone, butanone, pentanone, heptanone, hydroxyacetone and acetylacetone) and formate. The peak for the desired product is indicated by the arrow.

FIG. 3 shows specific ketone substrates for condensation with formyl-CoA that can be catalyzed by 2-hydroxy-acyl-CoA-synthase (HACS) to the corresponding product (a branched 2-hydroxyacid).

FIG. 4 shows pathways for the production of branched 2-hydroxyacid, branched 3-hydroxyacid, branched alcohol, branched 1,2-diol and branched ab-unsaturated acid from the branched 2-hydroxy-Cₙ₊₁-acyl-CoA produced by condensation of carboxylic acid-derived a Cₙ-ketone (derived from a carboxylic acid) and formyl-CoA.

FIG. 5 shows exemplary ketones that can be used as substrates for condensation with formyl-CoA (catalyzed by HACS) to produce a branched 2-hydroxyacyl-CoA and the corresponding branched chain products (2-hydroxyacid, 3-hydroxyacid, carboxylic acid, ab-unsaturated acid, 1,2-diol, and alcohol) that can be produced from the branched 2-hydroxyacyl-CoA.

FIG. 6A shows pathways for the production of 2-hydroxyisobutyric acid (2HIB), 3-hydroxyisobutyric acid, isobutanol, isobutene glycol and methacrylic acid from the condensation of lactic acid-derived acetone and formyl-CoA (which produces 2-hydroxyisobutyryl-CoA).

FIG. 6B shows the screening results of first round HACS for acetone-C1 condensation reaction represented by 2-hydroxyisobutyric acid (2HIB) titer.

FIG. 6C shows the screening results of second round HACS for acetone-C1 condensation reaction using ApbHACS as a reference.

FIG. 7A shows pathways for the production of 2-hydroxy-2-methylbutanoic acid, 3-hydroxy-2-methylbutanoic acid, 2-methylbutan-1-ol, 2-methylbutane-1,2-diol and 2-methylbut-2-enoic acid from condensation of 2-hydroxybutanoic acid derived butanone and formyl-CoA.

FIG. 7B shows screening results of second round HACS for butanone-C1 condensation using ApbHACS as a reference.

FIG. 8 shows pathways for the production of 2-hydroxy-2-methylpentanoic acid, 3-hydroxy-2-methylpentanoic acid, 2-methylpentan-1-ol, 2-methylpentane-1,2-diol and 2-methylpen-2-enoic acid from condensation of 2-hydroxypentanoic acid-derived pentanone and formyl-CoA.

FIG. 9 shows pathways for the production of 2-hydroxy-2-methylheptanoic acid, 3-hydroxy-2-methylheptanoic acid, 2-methylheptan-1-ol, 2-methylheptane-1,2-diol and 2-methylhept-2-enoic acid from the condensation of 2-hydroxyheptanoic acid-derived heptanone and formyl-CoA.

FIG. 10 shows pathways for the production of 2,3-hydroxy-2-methylpropanoic acid, 2-methylpropane-1,3-diol, 2-methylpropane-1,2,3-triol and 3-hydroxy-2-methylacrylic acid from the condensation of 2,3-hydroxypropanoic acid-derived hydroxyacetone and formyl-CoA.

FIG. 11 shows pathways for the production of 2-hydroxy-2,3-dimethylbutanoic acid, 3-hydroxy-2,3-dimethylbutanoic acid, 2,3-dimethylbutan-1-ol, 2,3-dimethylbutane-1,2-diol and 2,3-dimethylbut-2-enoic acid from the condensation of 2-hydroxy-3methylbutanoic acid-derived 3-methyl-2-butanone and formyl-CoA.

FIG. 12 shows pathways for the production of 2-hydroxy-2-methyl-3-oxopropanoic acid, 2-methylpropane-1,3-diol, and 2-methylheptane-1,2,3-triol from the condensation of 2-hydroxy-3-oxopropanoic acid-derived methylglyoxal and formyl-CoA.

FIG. 13 shows pathways for the production of 2-hydroxy-2-methyl-4-oxopentanoic acid, 3-hydroxy-2-methyl-4-oxopentanoic acid, 5-hydroxy-4-methylpentan-2-one, 4,5-dihydroxy-4-methylpentan-2-one and 2-methyl-4-oxopent-2-enoic acid from condensation of 2-hydroxy-4-oxopentanoic acid-derived pentane-2,4-dione and formyl-CoA.

FIGS. 14A, 14B, 14C, and 14D demonstrate C1-C1 (formaldehyde-formyl-CoA) condensation for product synthesis in vivo.

FIGS. 15A, 15B and 15C demonstrate a scheme for the production of acetone from glucose. FIG. 15A summarizes the pathway for the production of acetone from glucose.

FIGS. 15B and 15C are bar graphs showing acetone titer after 2 days and 5 days of fermentation, respectively.

FIGS. 16A, 16B and 16C demonstrate the production of acetone from acetate in AC440 host cultured in M9-LB in the presence of sodium acetate and 500 mM of methanol.

FIG. 16A summarizes the scheme for the production of acetone from acetate. FIGS. 16B and 16C are bar graphs showing acetone titer (FIG. 16B) and residual acetate (FIG. 16C) after 1 days and 3 days of fermentation as shown.

FIG. 17A shows a scheme and bar graph for the production of 2-hydroxyisobutyrate (2HIB) from acetone and formaldehyde using the indicated HACS enzymes.

FIG. 17B shows a scheme and bar graph for the production of 2-hydroxyisobutyrate (2HIB) from acetone and formate using the indicated HACS enzymes.

FIG. 18A shows a scheme and bar graph for the production of 2-hydroxyisobutyrate (2HIB) from acetone and formate using the indicated HACS enzymes using growing cells (AC440 host).

FIG. 18B shows a scheme and bar graph for the production of 2-hydroxyisobutyrate (2HIB) from acetone and formaldehyde using the indicated HACS enzymes using growing cells (FZ635 host).

FIG. 18C shows a scheme and bar graph for the production of 2-hydroxyisobutyrate (2HIB) from acetone and methanol using the indicated HACS enzymes using growing cells (CAL124 host).

FIG. 19 is a graph showing 2HIB production from formate and acetone in a bioreactor.

FIGS. 20A and 20B demonstrates isobutanol production from the condensation of acetone and formyl-CoA.

FIG. 21 is a graph showing 2HIB tolerance in the AC440 host.

FIG. 22 shows a generic scheme for energy (redox and ATP) and formyl-CoA generation.

FIGS. 23A and 23B show the screening of formate activation enzymes (FAEs) for formyl-CoA generation. FIG. 23A shows a pathway for screening FAE platform for formyl-CoA generation form formate with formaldehyde as a co-substrate. FIG. 23B shows screening results of the FAE candidates with formaldehyde and formate using resting cells.

FIG. 23C shows a pathway for screening FAEs for formyl-CoA generation from formate with acetone as a co-substrate. FIG. 23D shows the screening results of FAE candidates using acetone and formate using growing cultures.

FIGS. 24A and 24B shows the screening of enzymes for formaldehyde to formyl-CoA using glycolate production as a proxy.

FIGS. 25A-25C show a scheme for methanol to energy (redox and ATP) and formyl-CoA.

FIGS. 26A and 26B shows pathway prototyping using formaldehyde as a starting substrate.

FIGS. 27A and 27B shows a pathway from glycolate to glycine.

FIG. 28 shows a pathway using formate to formamide. Table 7 provides additional information.

FIG. 29 shows an expression vector expressing formamidase (MmFmdA).

FIG. 30 shows the production of branched 2-hydroxyacid, 3-hydroxyacid, alcohol, 1,2-diol and a,b-unsaturated acid from condensation of carboxylic acid-derived aldehyde and formyl-CoA.

FIG. 31A shows a carboxylic acid platform using acetic acid as the substrate.

FIG. 31B shows a graph of HACS screening results for acetaldehyde-C1 condensation reaction represented by lactic acid productivity.

FIG. 32A shows a carboxylic acid platform using glycolic acid as the substrate.

FIG. 32B shows a graph of HACS screening results for a glycoaldehyde-C1 condensation reaction represented by glyceric acid.

FIG. 33A shows a carboxylic acid platform using succinic acid as the substrate.

FIG. 33B shows a graph of HACS screening results for a semialdehyde-C1 condensation reaction represented by 2-hydroxyglutaric acid.

FIG. 34A shows a carboxylic acid platform using isovaleric acid as the substrate.

FIG. 34B shows a graph of HACS screening results for an isovaleraldehyde-C1 condensation reaction represented by leucic acid.

FIG. 35A shows a carboxylic acid platform using 3-(methylthio)propionic acid as the substrate.

FIG. 35B shows a graph of HACS screening results for a methional-C1 condensation reaction represented by desmeninol productivity.

FIG. 36 shows a carboxylic acid platform using hydroxypivalic acid as the substrate.

### DETAILED DESCRIPTION OF THE INVENTION

A description of preferred embodiments of the invention follows.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It must be noted that, as used in the specification and the appended claims, the singular forms “a,” “an,” and “the” include plural referents unless the context clearly dictates otherwise. Thus, for example, reference to “an enzyme” includes a plurality of enzymes.

As defined herein, the phrases “recombinant host microorganism”, “genetically engineered host microorganism”, “engineered microorganism” and “genetically modified microorganism” and the like may be used interchangeably and refer to host microorganisms that have been genetically modified to (a) express one or more exogenous or heterologous polynucleotides or DNAs, (b) over-express one or more endogenous and/or one or more exogenous or heterologous polynucleotides or DNAs, such as those included in a vector, or which have an alteration in expression of an endogenous gene or (c) knock-out or down-regulate an endogenous gene. In addition, certain genes may be physically removed from the genome (e.g., knock-outs) or they may be engineered to have reduced, altered or enhanced activity.

In certain aspects, the microorganism is E. coli. Additional exemplary bacteria include, e.g., Bacillus, Streptomyces, Azotobacter, Rhizobium, Pseudomonas, Micrococcus, Nitrobacter, Proteus, Lactobacillus, Pediococcus, Lactococcus, Salmonella, Streptococcus, Paracoccus, Vibrio, Corynebacterium, Methanosarcina, Methylococcus, Methylobacterium, Methylomicrobium, Synechococcus, Rhodobacter or any of the completely sequenced bacterial species. Indeed, hundreds of bacterial genomes have been completely sequenced, and this information greatly simplifies both the generation of vectors encoding the needed genes, as well as the planning of a recombinant engineering protocol. Such species are listed along with links at en.wikipedia.org/wiki/List_of_sequenced_bacterial_genomes. Additionally, yeast, such as Saccharomyces are common species used for microbial manufacturing, and many species can be successfully engineered with heterologous metabolic pathways for product synthesis. Other species include but are not limited to Candida, Arxula adeninivorans, Candida boidinii, Hansenula polymorpha (Pichia angusta), Kluyveromyces lactis, Pichia pastoris, and Yarrowia lipolytica, to name a few. It is also possible to genetically modify many species of algae, including e.g., Spirulina, Chlamydomonas, Laminaria japonica, Undaria pinnatifida, Porphyra, Eucheuma, Kappaphycus, Gracilaria, Monostroma, Enteromorpha, Arthrospira, Chlorella, Dunaliella, Aphanizomenon, Isochrysis, Pavlova, Phaeodactylum, Ulkenia, Haematococcus, Chaetoceros, Nannochloropsis, Skeletonema, Thalassiosira, Botryococcus and Laminaria japonica. Indeed, the microalga Pavlova lutheri is already being used as a source of economically valuable docosahexaenoic (DHA) and eicosapentaenoic acids (EPA), and Crypthecodinium cohnii is the heterotrophic algal species that is currently used to produce the DHA used in many infant formulas. Non-limiting examples of microorganisms that can be used include Escherichia coli, Saccharomyces cerevisiae, Bacillus methanolicus, Pichia pastoris, Candida boidinii, Pseudomonas putida, Methylococcus capsulatus, Methylobacterium extorquens, Methylomicrobium buryatense, Corynebacterium glutamicum, Clostridium autoethanogenum, and Clostridium ljungdahlii. Furthermore, a number of databases include vector information and/or a repository of vectors and can be used to choose vectors suitable for the chosen host species. See e.g., AddGene.org which provides both a repository and a searchable database allowing vectors to be easily located and obtained from colleagues. See also Plasmid Information Database (PlasmID) and DNASU having over 191,000 plasmids. A collection of cloning vectors of E. coli is also kept at the National Institute of Genetics as a resource for the biological research community. Furthermore, vectors (including particular ORFS therein) are usually available from colleagues.

The terms “engineer”, “genetically engineer” or “genetically modify” and the like refer to any manipulation of a microorganism that results in a detectable change in the microorganism, wherein the manipulation includes, but is not limited to, introducing non-native metabolic functionality via heterologous (exogenous) polynucleotides or removing native-functionality via polynucleotide deletions, mutations or knock-outs. The term “metabolically engineered” generally involves rational pathway design and assembly of biosynthetic genes (or ORFs), genes associated with operons, and control elements of such polynucleotides, for the production of a desired metabolite. “Metabolically engineered” may further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway.

The enzymes can be added to the genome or via expression vectors, as desired. Preferably, multiple enzymes are expressed in one vector or multiple enzymes can be combined into one operon by adding the needed signals between coding regions. Further improvements can be had by overexpressing one or more, or even all of the enzymes, e.g., by adding extra copies to the cell via plasmid or other vector. Initial experiments may employ expression plasmids hosting 3 or more ORFs for convenience, but it may be preferred to insert operons or individual genes into the genome for stability reasons.

Still further improvements in yield can be had by reducing competing pathways. In certain examples, pathways for making e.g., acetate, formate, ethanol, and lactate can be reduced, and it is already well known in the art how to reduce or knockout these pathways. See e.g., the William Marsh Rice University patent portfolio by Ka-Yiu San and George Bennett (U.S. Pat. Nos. 7,569,380, 7,262,046, 8,962,272, 8,795,991) and patents by these inventors (U.S. Pat. Nos. 8,129,157 and 8,691,552) (each incorporated by reference herein in its entirety for all purposes). Many others have worked in this area as well.

The term “mutation” as used herein indicates any modification of a nucleic acid and/or polypeptide which results in an altered nucleic acid or polypeptide (i.e., relative to the wild-type nucleic acid or polypeptide sequence). Mutations include, for example, point mutations, substitutions, deletions, or insertions of single or multiple residues in a polynucleotide (or the encoded polypeptide), which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In certain embodiments, a portion of a genetically modified microorganism's genome may be replaced with one or more heterologous (exogenous) polynucleotides. In some embodiments, the mutations are naturally-occurring. In other embodiments, the mutations are the results of artificial selection pressure. In still other embodiments, the mutations in the microorganism genome are the result of genetic engineering.

The term “expression” or “expressed” with respect to a gene sequence, an ORF sequence or polynucleotide sequence, refers to transcription of the gene, ORF or polynucleotide and, as appropriate, translation of the resulting mRNA transcript to a protein. Thus, as will be clear from the context, expression of a protein results from transcription and translation of the open reading frame sequence. The level of expression of a desired product in a host microorganism may be determined on the basis of either the amount of corresponding mRNA that is present in the host, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press, 1989). Protein encoded by a selected sequence can be quantitated by various methods (e.g., by ELISA, by assaying for the biological activity of the protein, or by employing assays that are independent of such activity, such as western blotting or radioimmunoassay, using antibodies that are recognize and bind reacting the protein).

“Overexpression” or “overexpressed” is defined herein to be at least 150% of protein activity as compared with an appropriate control species, or any expression in a species that lacks the activity altogether. Preferably, the activity is increased 100-500%. Overexpression can be achieved, for example, by mutating the protein to produce a more active form or a form that is resistant to inhibition, by removing inhibitors, or adding activators, and the like. Overexpression can also be achieved by removing repressors, adding multiple copies of the gene to the cell, or up-regulating the endogenous gene, and the like.

The term “endogenous”, as used herein with reference to polynucleotides (and the polypeptides encoded therein), indicates polynucleotides and polypeptides that are expressed in the organism in which they originated (i.e., they are innate to the organism). In contrast, the terms “heterologous” and “exogenous” are used interchangeably, and as defined herein with reference to polynucleotides (and the polypeptides encoded therein), indicates polynucleotides and polypeptides that are expressed in an organism other than the organism from which they (i.e., the polynucleotide or polypeptide sequences) originated or where derived.

The term “feedstock” is defined as a raw material or mixture of raw materials supplied to a microorganism, or fermentation process, from which other products can be made. However, in addition to a feedstock, the fermentation media contains suitable minerals, salts, cofactors, buffers and other components, known to those skilled in the art, suitable for the growth of the cultures and promotion of the enzymatic pathways necessary for production of the products described herein.

The term “substrate” refers to any substance or compound that is converted, or meant to be converted, into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material (e.g., methane), but also intermediate and end product metabolites used in a pathway associated with a metabolically engineered microorganism as described herein.

The term “fermentation” or “fermentation process” is defined as a process in which a host microorganism is cultivated in a culture medium containing raw materials, such as feedstock and nutrients, wherein the microorganism converts raw materials, such as a feedstock, into products.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) of more codons representing amino acids, and (iii) a termination codon, the ORF being read (or translated) in the 5′ to 3′ direction.

It is understood that the polynucleotides described herein include “genes” and that the nucleic acid molecules described herein include “vectors” or “plasmids”.

Accordingly, the term “gene”, refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “promoter” refers to a nucleic acid sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “codon-optimized” as it refers to genes or coding regions of nucleic acid molecules (or ORFs) for transformation of various hosts, refers to the alteration of codons in the gene or coding regions of the nucleic acid molecules to reflect the typical codon usage of the host organism without altering the polypeptide encoded by the DNA.

The term “operon” refers to two or more genes which are transcribed as a single transcriptional unit from a common promoter. In certain embodiments, the genes, polynucleotides or ORFs comprising the operon are contiguous genes. It is understood that transcription of an entire operon can be modified (i.e., increased, decreased, or eliminated) by modifying the common promoter. Alternatively, any gene, polynucleotide or ORF, or any combination thereof in an operon can be modified to alter the function or activity of the encoded polypeptide. The modification can result in an increase or a decrease in the activity or function of the encoded polypeptide. Further, the modification can impart new activities on the encoded polypeptide.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes”, that is, that replicate autonomously or can integrate into a chromosome of a host microorganism. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

The term “homolog”, as used with respect to an original enzyme, polypeptide, gene or polynucleotide (or ORF encoding the same) of a first family or species, refers to distinct enzymes, genes or polynucleotides of a second family or species, which are determined by functional, structural or genomic analyses to be an enzyme, gene or polynucleotide of the second family or species, which corresponds to the original enzyme or gene of the first family or species. Most often, “homologs” will have functional, structural or genomic similarities. Techniques are known by which homologs of an enzyme, gene or polynucleotide can readily be cloned using genetic probes and PCR. Identity of cloned sequences as “homologs” can be confirmed using functional assays and/or by genomic mapping of the genes.

A polypeptide (or protein or enzyme) has “homology” or is “homologous” to a second polypeptide if the nucleic acid sequence that encodes the polypeptide has a similar sequence to the nucleic acid sequence that encodes the second polypeptide.

Alternatively, a polypeptide has homology to a second polypeptide if the two proteins have “similar” amino acid sequences. Thus, the terms “homologous proteins” or “homologous polypeptides” is defined to mean that the two polypeptides have similar amino acid sequences. In certain embodiments of the invention, polynucleotides and polypeptides homologous to one or more polynucleotides and/or polypeptides set forth in Table 1 may be readily identified using methods known in the art for sequence analysis and comparison.

A homologous polynucleotide or polypeptide sequence of the invention may also be determined or identified by BLAST analysis (Basic Local Alignment Search Tool) or similar bioinformatic tools, which compare a query nucleotide or polypeptide sequence to a database of known sequences. For example, a search analysis may be done using BLAST to determine sequence identity or similarity to previously published sequences, and if the sequence has not yet been published, can give relevant insight into the function of the DNA or protein sequence.

As used herein, “carbon dioxide reductase” (EC 1.2.1.2), catalyzes the formation of formate from carbon dioxide (CO₂).

As used herein, “formate kinase” (EC 2.7.2.6), catalyzes the formation of formyl-phosphate from formate.

As used herein, “phosphate formyl-transferase” (EC 2.3.1.8), catalyzes the formation of formyl-CoA from formyl-phosphate.

As used herein, “acyl-CoA synthetase” (EC 6.2.1.−), catalyzes the formation of an acyl-CoA from a respective carboxylic acid or associated anion (e.g. formic acid or formate).

A “synthase” is a generic term that describes an enzyme that catalyzes the synthesis of a biological compound without the requirement of a nucleoside triphosphate, such as ATP, as co-substrate. A “synthetase” catalyzes the synthesis of a biological compound and requires ATP or another nucleoside triphosphate cosubstrate. “Ligase” is a term that describes an enzyme that catalyzes condensation of biological molecules optionally with simultaneous cleavage of ATP or other high energy substrate. However, in the scientific literature the terms “synthase”, “synthetase”, and “ligase” do not always follow these strict definitions. Accordingly, as used herein, these terms may be used interchangeably.

As used herein, “methane monooxygenase” (EC 1.14.13.25; 1.14.18.3), catalyzes the formation of methanol from methane.

As used herein, “methanol dehydrogenase” (EC 1.1.1.244; 1.1.2.7; 1.1.99.37), catalyzes the formation of formaldehyde from methanol.

As used herein, “acyl-CoA reductase” or “acylating aldehyde dehydrogenase” (EC 1.2.1.10), catalyzes the formation of an acyl-CoA from a respective aldehyde (e.g. formaldehyde) or the reverse.

As used herein, “thioesterase” (EC 3.1.2.−), catalyzes the formation of a carboxylic acid from a respective acyl-CoA.

As used herein, “alcohol dehydrogenase” (EC 1.1.1.−), catalyzes the conversion of an aldehyde functional group to an alcohol.

Abbreviations used herein include HACS: 2-hydroxyacyl-CoA synthase; MDH: methanol dehydrogenase; ACR: acyl-CoA reductase; HACL: 2-hydroxyacyl-CoA lyase; ADH: alcohol dehydrogenase; DDR: diol dehydratase; TES: thioesterase; ACT: acyl-CoA transferase; PTA: phosphotransacylase (PTA); CAK: carboxylate kinase.

It will be appreciated that the description of the chemical formulae herein should be construed in congruity with the laws and principals of chemical bonding.

“Alkyl” refers to a straight or branched hydrocarbon chain radical consisting solely of carbon and hydrogen atoms, containing no unsaturation and which is attached to the rest of the molecule by a single bond. In some embodiments, an alkyl group has from one to twelve carbon atoms, one to eight carbon atoms, or one to six carbon atoms. Non-limiting examples of alkyl groups include methyl, ethyl, n-propyl, 1-methylethyl (isopropyl), n-butyl, n-pentyl, 1,1-dimethylethyl (t-butyl), 3-methylhexyl, 2-methylhexyl, and the like. An optionally substituted alkyl group can by an alkyl group substituted with one or more substituents described in detail below. Non-limiting examples of optionally substituted alkyls include haloalkyl, alkyl substituted with cyano, optionally substituted aralkyl, optionally substituted heteroarylalkyl, optionally substituted cycloalklylalkyl, optionally substituted heterocycloalkyl, alkyl substituted with an amino group, alkyls substituted with hydroxyl or alkoxy, and the like.

“Alkenyl” refers to a straight or branched hydrocarbon chain radical group consisting solely of carbon and hydrogen atoms, containing at least one double bond. In some cases, an alkenyl can have from two to twelve carbon atoms, or two to eight carbon atoms. An alkenyl is attached to the rest of the molecule by a single bond, for example, ethenyl, prop-1-enyl, but-1-enyl, pent-1-enyl, penta-1,4-dienyl, and the like. An optionally substituted alkenyl group can be an alkyl group substituted with one or more substituents described in detail below.

“Alkynyl” refers to a straight or branched hydrocarbon chain radical group consisting solely of carbon and hydrogen atoms, containing at least one triple bond, optionally containing at least one double bond. In some embodiments, an alkynyl can have from two to twelve carbon atoms, or two to eight carbon atoms. An alkynyl is attached to the rest of the molecule by a single bond, for example, ethynyl, propynyl, butynyl, pentynyl, hexynyl, and the like. An optionally substituted alkynyl group can by an alkyl group substituted with one or more substituents described in detail below.

“Aryl” refers to aromatic monocyclic or multicyclic hydrocarbon ring system consisting only of hydrogen and carbon and containing from 6 to 19 carbon atoms. Aryl groups include, but are not limited to, groups such as fluorenyl, phenyl and naphthyl. In certain aspect, the aryl is phenyl.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, and which is saturated or unsaturated and attached to the rest of the molecule by a single bond. In some aspects, a cycloalkyl will have from three to ten carbon atoms. Monocyclic radicals include, for example, cyclopropyl, cyclobutyl, cyclopentyl, cyclohexyl, cycloheptyl, and cyclooctyl. Polycyclic radicals include, for example, adamantine, norbornane, 7,7-dimethyl-bicyclo[2.2.1]heptanyl, and the like.

“Cycloalkenyl” refers to monocyclic or polycyclic hydrocarbon alkenyl moiety having 3 to fifteen carbon atoms.

The term “cycloalkynyl,” refers to a monocyclic or polycyclic alkynyl moiety having 5 to 15 more carbon atoms.

“Halo” refers to bromo, chloro, fluoro or iodo.

“Haloalkyl” refers to an alkyl radical, as defined above, that is substituted by one or more halo radicals, as defined above, for example, trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted.

“Haloalkenyl” refers to an alkenyl radical, as defined above, that is substituted by one or more halo radicals, as defined above. The alkenyl part of the haloalkyl radical may be optionally substituted. “Haloalkynyl” refers to an alkynyl radical, as defined above, that is substituted by one or more halo radicals, as defined above. The alkynyl part of the haloalkyl radical may be optionally substituted.

“Heterocyclyl” and “heterocyclic” refer to a stable 3- to 18-membered non-aromatic ring radical which includes one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, azepinyl, 2,5-diazabicyclo[2.2.1]heptan-2-yl, hexahydro-1H-1,4-diazepinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxiranyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heteroaryl” refers to a 3- to 18-membered fully or partially aromatic ring radical which consists of one to thirteen carbon atoms and from one to six heteroatoms selected from the group consisting of nitrogen, oxygen and sulfur. For purposes of this invention, the heteroaryl radical may be a monocyclic, bicyclic, tricyclic or tetracyclic ring system, which may include fused or bridged ring systems; the nitrogen, carbon or sulfur atoms in the heteroaryl radical may be optionally oxidized; and the nitrogen atom may be optionally quaternized. Examples include, but are not limited to, acridinyl, benzimidazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, indolizinyl, isoxazolyl, naphthyridinyl, oxadiazolyl, 2-oxoazepinyl, oxazolyl, phenazinyl, phenothiazinyl, phenoxazinyl, phthalazinyl, pteridinyl, purinyl, pyrazolyl, pyridinyl, pyrazinyl, pyrimidinyl, pyridazinyl, pyrrolyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e. thienyl).

The term “substituted” refers to substitution by independent replacement of one, two, or three or more of the hydrogen atoms with substituents including, but not limited to, —C₁-C₁₂ alkyl, —C₂-C₁₂ alkenyl, —C₂-C₁₂ alkynyl, —C₃-C₁₅ cycloalkyl, —C₃-C₁₅ cycloalkenyl, C₃-C₁₅ cycloalkynyl, -heterocyclic, —F, —Cl, —Br, —I, —OH, —NO₂, —N₃, —CN, —NH₂, oxo, thioxo, —NHRₓ, —NRₓRₓ, dialkylamino, -diarylamino, -diheteroarylamino, —ORₓ, —C(O)OR_{y}, —C(O)R_{y}, —C(O)C(O)R_{y}, —OCO₂R_{y}, —OC(O)R_{y}, OC(O)C(O)R_{y}, —NHC(O)R_{y}, —NHCO₂R_{y}, —NHC(O)C(O)R_{y}, —NHC(S)NH₂, —NHC(S)NHRₓ, —NHC(NH)NH₂, —NHC(NH)NHRₓ, —NHC(NH)Rₓ, —C(NH)NHRₓ, —NRₓC(O)Rₓ, —NRₓCO₂R_{y}, —NRₓC(O)C(O)R_{y}, —NRₓC(S)NH₂, —NRₓC(O)NRₓRₓ, —NRₓS(O)₂NRₓRₓ, —NRₓC(S)NHRₓ, —NRₓC(NH)NH₂, —NRₓC(NH)NHRₓ, —NRₓC(NH)Rₓ, —C(NRₓ)NHRₓ, —S(O)ₙR_{y}, —NHSO₂Rₓ, —CH₂NH₂, —CH₂SO₂CH₃, —(C═NRₓ)Rₓ; -aryl, -arylalkyl, -heteroaryl, -heteroarylalkyl, -heterocycloalkyl, —C₃-C₁₅-cycloalkyl, -polyalkoxyalkyl, -polyalkoxy, -methoxymethoxy, -methoxyethoxy, —SH, —S—Rₓ, or -methylthiomethyl, wherein Rₓ is selected from the group consisting of hydrogen, —C₁-C₁₂ alkyl, —C₂-C₁₂ alkenyl, —C₂-C₁₂ alkynyl, —C₃-C₁₅ cycloalkyl, -aryl, -heteroaryl and -heterocyclic; —R_{y} is selected from the group consisting of hydrogen, —C₁-C₁₂ alkyl, —C₂-C₁₂ alkenyl, —C₂-C₁₂ alkynyl, —C₃-C₁₅ cycloalkyl, -aryl, -heteroaryl, -heterocyclic, —NH₂, —NH—C₁-C₁₂ alkyl, —NH—C₂-C₁₂ alkenyl, —NH—C₂-C₁₂-alkynyl, —NH—C₃-C₁₅ cycloalkyl, —NH-aryl, —NH-heteroaryl and —NH-heterocyclic, and n is 0, 1 or 2. It is understood that the aryls, heteroaryls, alkyls, cycloalkyls, heterocyclics and the like can be further substituted.

Methods for the condensation of formyl-CoA with certain aldehydes and ketones to produce 2-hydroxyacyl-CoAs, and methods for the preparation of the formyl-CoA and aldehyde and ketone substrates have been described in WO2016/069929A1, U.S. Pat. App. Pub. No. 20190100741A1, WO2020/247430, and WO2023287814, the contents of each of which are expressly incorporated by reference herein.

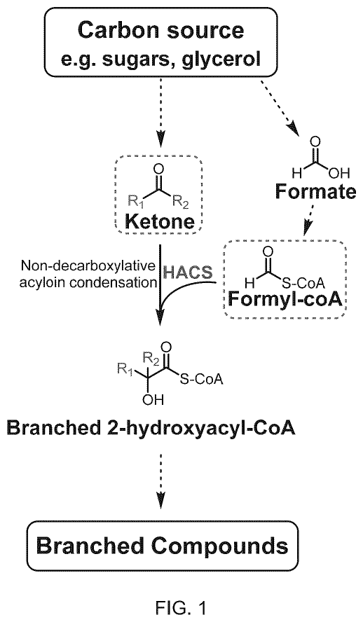

The present invention describes a method comprising the preparation of a branched 2-hydroxylacyl-CoA from formyl-CoA and a ketone, as well as a pathway for the preparation of branched chain products from the branched 2-hydroxyacyl-CoA. Described herein is a novel route for branched chain compounds production using a bio-based ketone as a co-substrate to condense with formyl-CoA to obtain a branched 2-hydroxyacyl-CoA (or in other words, with branched chain branched 2-hydroxyacyl-CoA) (FIG. 1). The branched 2-hydroxyacyl-CoA is converted to a branched chain compound, including but not limited to, a branched 2-hydroxyacid, a branched 3-hydroxyacid, a branched alcohol, a branched 1,2-diol and a branched ab-unsaturated acids. Ketones can be provided as a starting material or as a co-substrate, or the ketone can be generated from common carbon source, such as glucose and/or other biomass-derived sugars, glycerol, or acetate, for example. Such conversion of common carbon source to ketones have been demonstrated in numerous microorganisms. For example, a ketone can be produced from 2-hydroxy acid as mentioned above or through fatty acids synthesis or b-oxidation pathway demonstrated in the literatures (Appl Environ Microbiol 78:70-80, 2012; Metab Eng 62:84-94, 2020; the contents of which are expressly incorporated by reference herein).

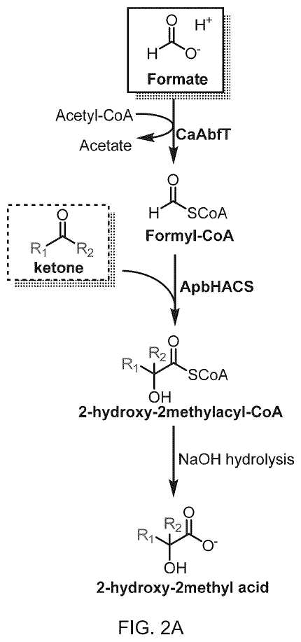

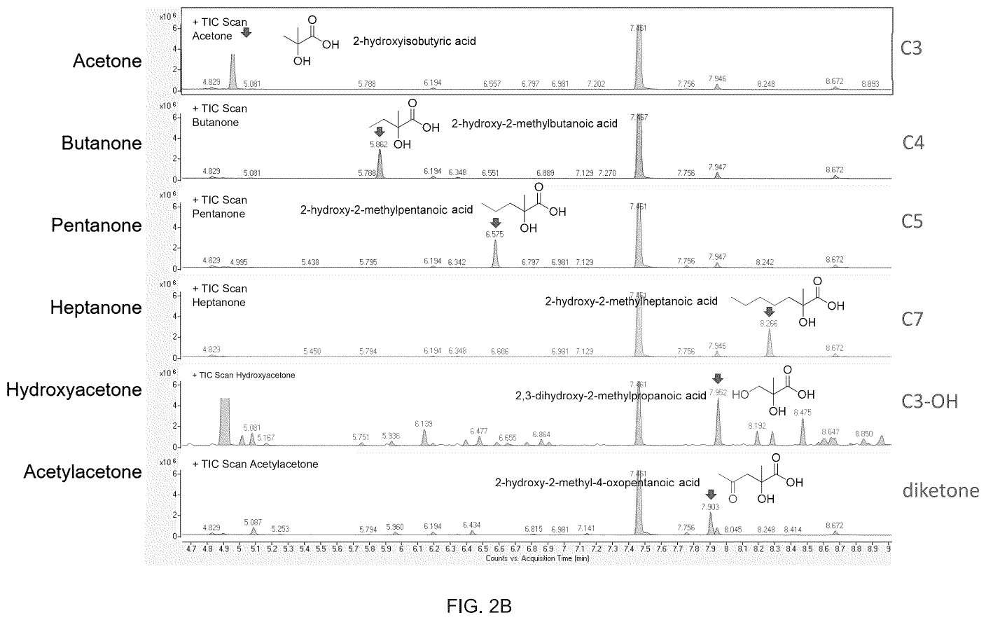

FIG. 2A shows an exemplary method of producing a branched 2-hydroxyacid as the branched chain product. Specifically, FIG. 2A shows that formate-derived formyl-CoA is condensed with methyl ketone in a reaction catalyzed by ApbHACS to produce 2-hydroxy-2-methylacyl-CoA. The 2-hydroxy-2-methylacyl-CoA is the hydrolyzed to produce the 2-hydroxy-2-methyl acid.

Non-limiting examples of ketones that can be used in the pathways described herein to produce branched chain products include methyl ketones, ethyl ketones, hydroxylated ketones (e.g. hydroxyacetone), and other ketones such as acetylacetone, branched-chain ketones, methylglyoxal. In certain aspects, the ketone can have the formula (I):

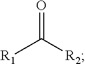

wherein:
R₁ and R₂ are each independently selected from the group consisting of C₁-C₁₂ alkyl, C₂-C₁₂ alkenyl, C₃-C₁₅ cycloalkenyl, aryl, heteroaryl, heterocyclyl, C(O)OR_{b}, C(O)R_{b}, SR_{b}, and N(R_{b})₂; wherein the C₁-C₁₂ alkyl, the C₂-C₁₂ alkenyl, the C₃-C₁₅ cycloalkenyl, the aryl, the heteroaryl, or the heterocyclyl are each optionally substituted by one or more Rₐ;
alternatively, R₁ and R₂ are taken together to form a C₃ to C₇ cycloalkyl, a C₃ to C₇ cycloalkenyl, or a 3- to 7-membered heterocyclyl, wherein the cycloalkyl, cycloalkenyl, or heterocyclyl are each optionally substituted by one or more Rₐ;
each Rₐ is independently selected from the group consisting of hydrogen, optionally substituted C₁-C₁₂ alkyl, optionally substituted C₂-C₁₂ alkenyl, optionally substituted C₂-C₁₂ alkynyl, optionally substituted C₃-C₁₅ cycloalkyl, optionally substituted C₃-C₁₅ cycloalkenyl, optionally substituted aryl, optionally substituted heteroaryl, optionally substituted heterocyclyl, C(O)R_{b}, C(O)OR_{b}, OR_{b}, N(R_{b})₂, halo, and oxo; and each R_{b} is independently hydrogen or a C₁-C₆ alkyl.
R₁ and R₂ can be the same or can be different. In certain aspects, R₂ is a C₁-C₆ alkyl, for example, R₂ can be methyl or ethyl. In certain aspects, R₂ is a C₁-C₆ alkyl and R₁ is a C₁-C₆ alkyl. In further aspects, R₁ and R₂ are each independently C₁-C₆ alkyl substituted by one or more Rₐ wherein the Rₐ is selected from the group consisting of hydrogen, OH and C(O)OH. In further examples, R₁ and R₂ are taken together to form a 3- to 7-membered heterocyclyl, wherein the heterocyclyl comprises a heteroatom selected from oxygen and nitrogen, and wherein the heterocyclyl is optionally substituted by one or more Rₐ.

In certain aspect, wherein when one of R₁ and R₂ is C(O)OR_{b} or C(O)R_{b}, the other of R₁ and R₂ is not C(O)OR_{b} or C(O)R_{b}.

Non-limiting examples of ketones that can be used in the pathways described herein are shown below in Table B:

TABLE B

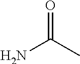

Acetamide

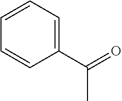

Acetophenone

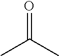

Acetone

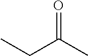

Butanone

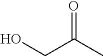

Hydroxyacetone

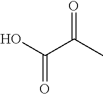

Pyruvic acid

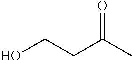

4-hydroxybutan-2-one

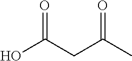

Acetoacetic acid

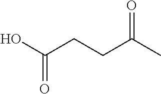

Levulinic acid

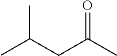

4-methylpentan-2-one

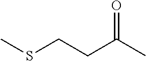

4-Methylthio-2-butanone

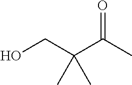

4-hydroxy-3,3-dimethylbutan-2-one

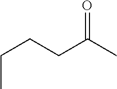

hexan-2-one

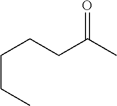

heptan-2-one

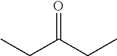

pentan-3-one

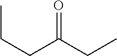

hexan-3-one

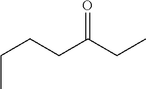

heptan-3-one

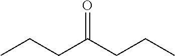

heptan-4-one

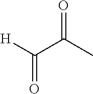

2-oxopropanal

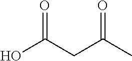

3-oxobutanoic acid
acetoacetate

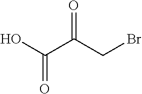

3-bromo-2-oxopropanoic acid

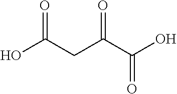

2-oxosuccinic acid
Oxaloacetate

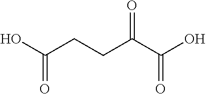

2-oxopentanedioic acid
a-Ketoglutaric acid

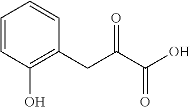 [SMILES: O=C(O)C(=O)Cc1ccccc1O]

3-(2-hydroxyphenyl)-2-oxopropanoic acid

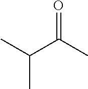

3-methylbutan-2-one

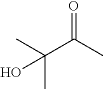

3-hydroxy-3-methylbutan-2-one

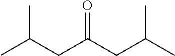

2,6-dimethylheptan-4-one
Diisobutyl ketone

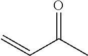

but-3-en-2-one

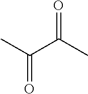

biacetyl

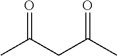

Acetylacetone

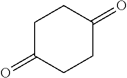

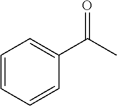

acetophenone

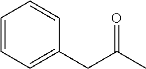

1-phenylpropan-2-one

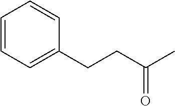

4-phenylbutan-2-one

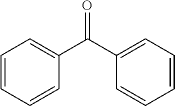 [SMILES: O=C(c1ccccc1)c1ccccc1]

benzophenone

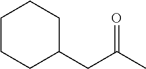

1-cyclohexylpropan-2-one

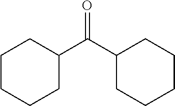 [SMILES: O=C(C1CCCCC1)C1CCCCC1]

Dicyclohexyl ketone

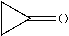

cyclopropanone

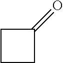

cyclobutanone

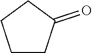

cyclopentanone

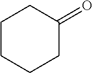

cyclohexanone

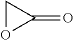

oxiran-2-one

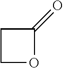

oxetan-2-one

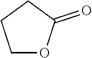

dihydrofuran-2(3H)-one

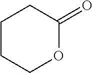

tetrahydro-2H-pyran-2-one

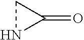

aziridin-2-one

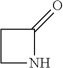

azetidin-2-one

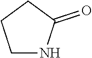

pyrrolidin-2-one

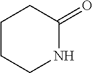

piperidin-2-one

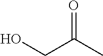

hydroxyacetone

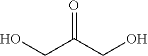

dihydroxyacetone

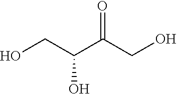

d-erythrulose

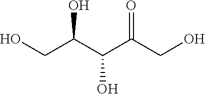

d-ribulose

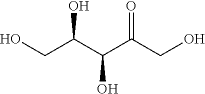

d-xylulose

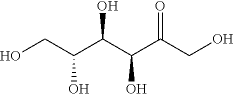 [SMILES: O=C(CO)[C@@H](O)[C@H](O)[C@H](O)CO]

d-fructose

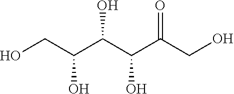 [SMILES: O=C(CO)[C@H](O)[C@@H](O)[C@H](O)CO]

d-sorbose

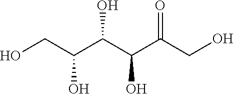 [SMILES: O=C(CO)[C@@H](O)[C@@H](O)[C@H](O)CO]

d-tagatose

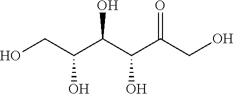 [SMILES: O=C(CO)[C@H](O)[C@H](O)[C@H](O)CO]

d-psicose

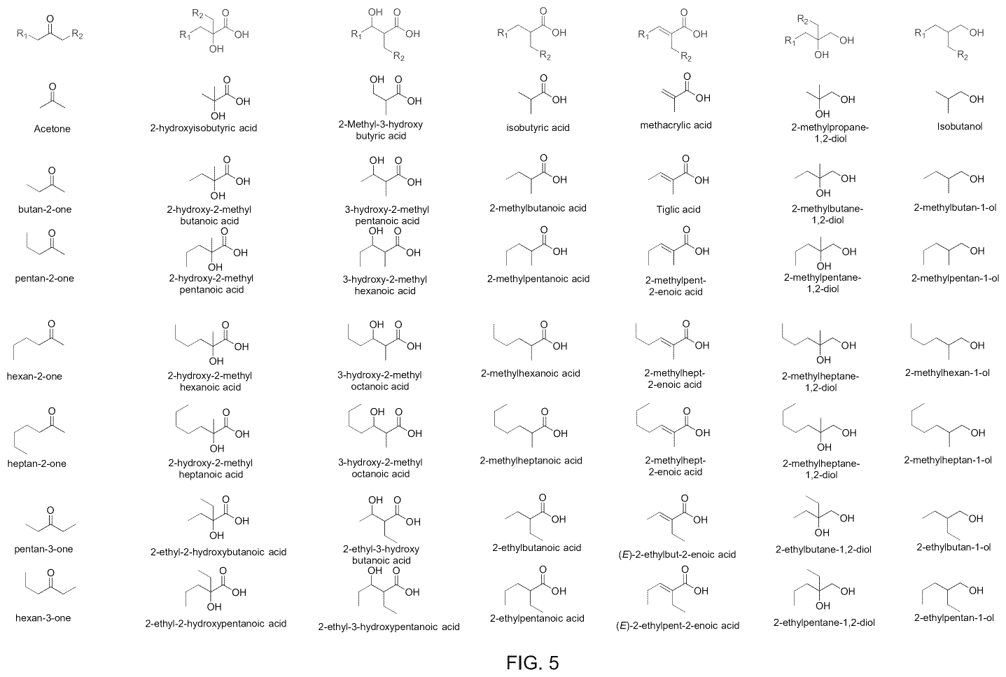

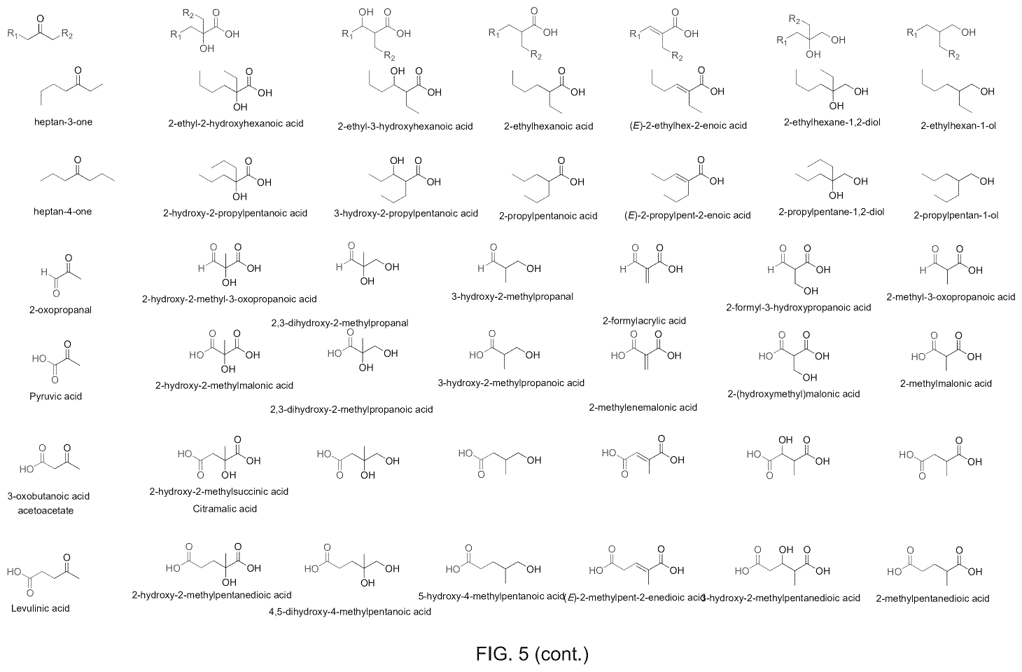

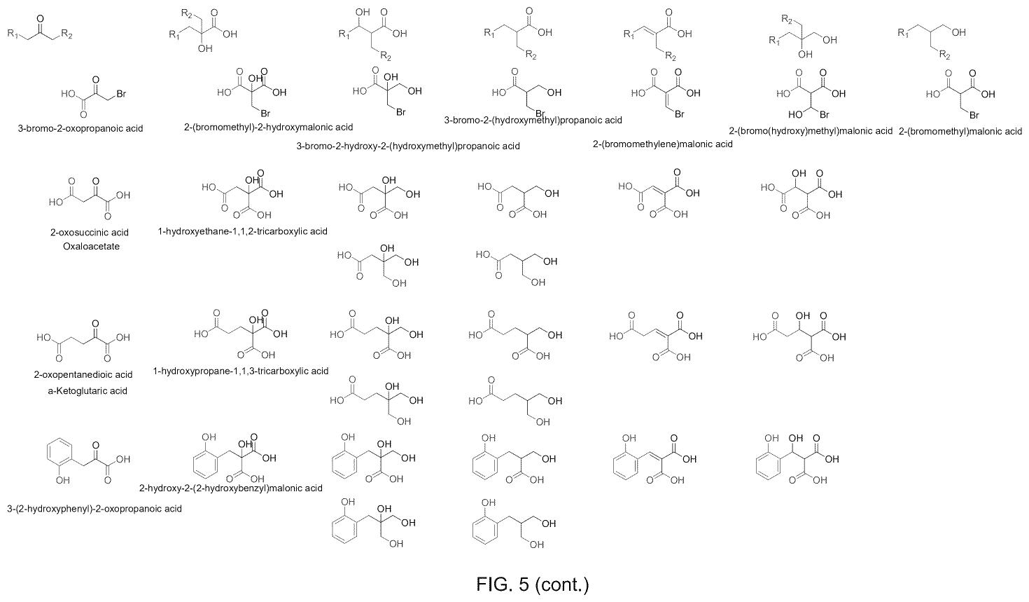

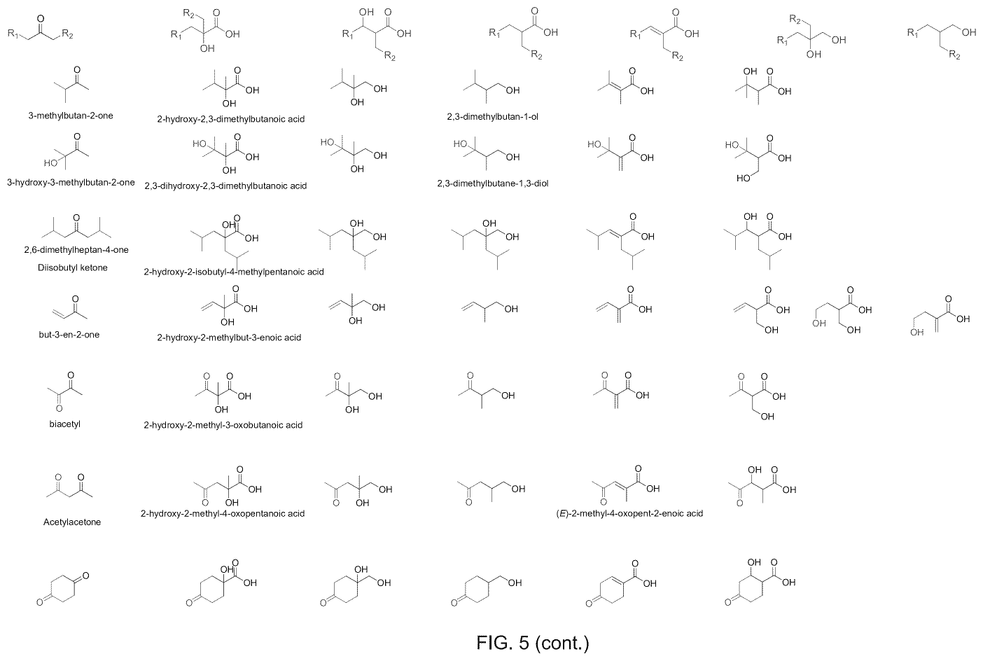

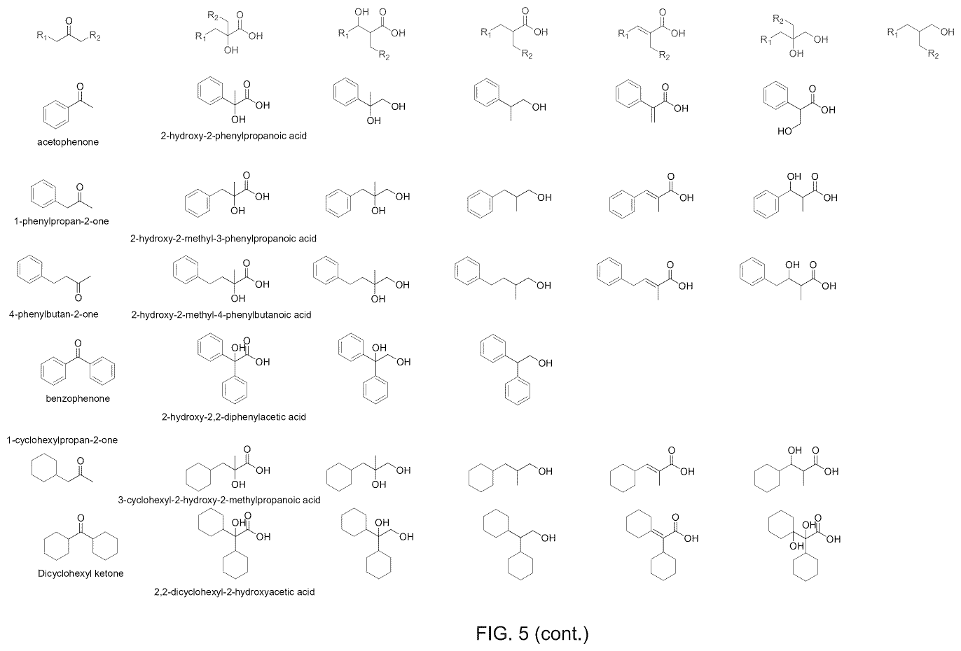

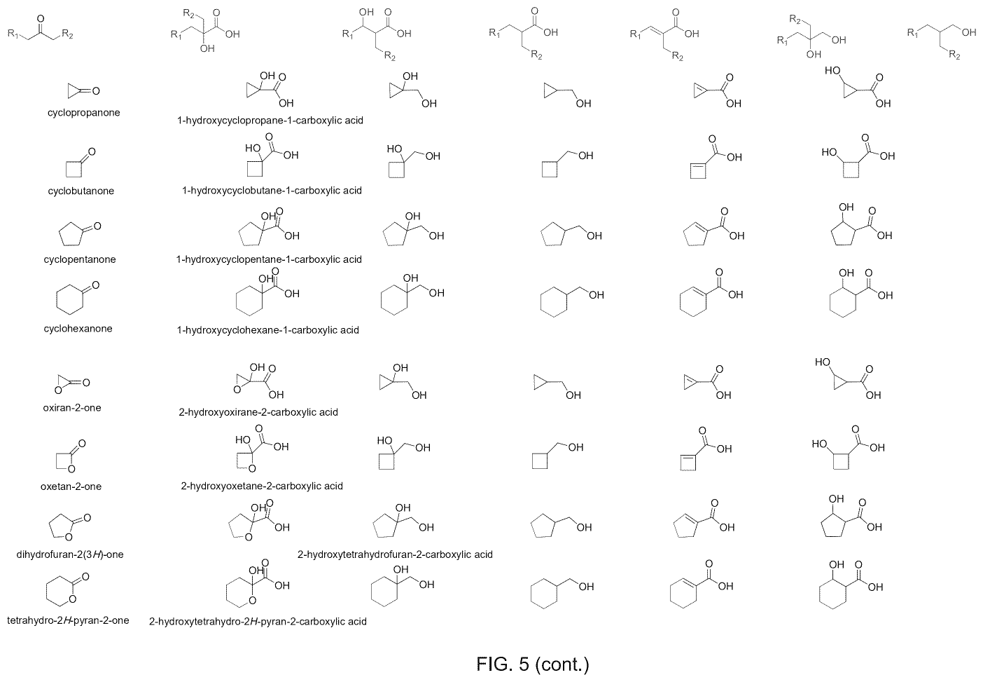

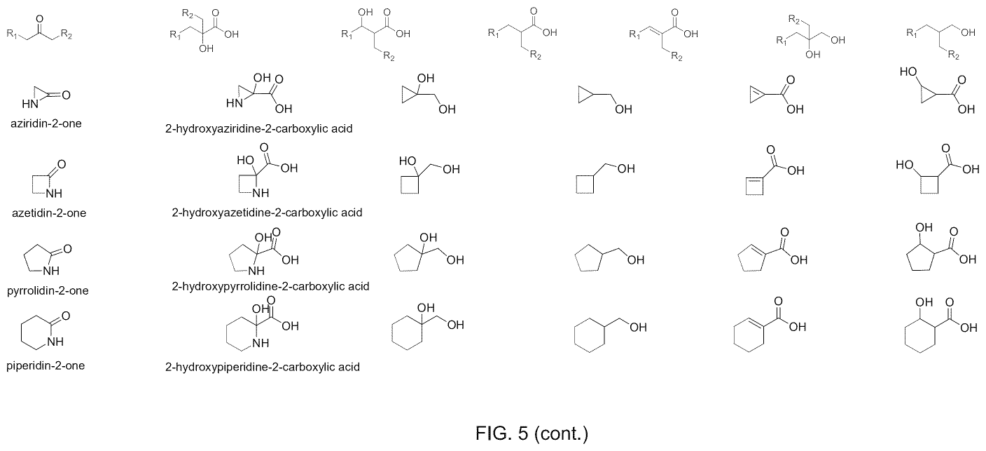

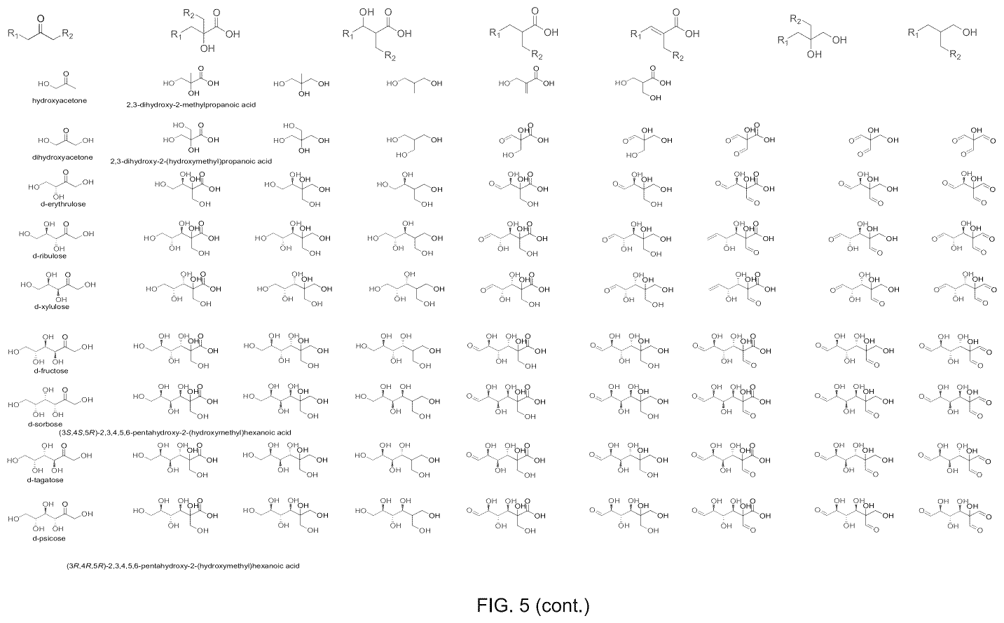

Yet additional examples are shown in FIG. 5.

The enzyme(s) that catalyzes the condensation of the ketone with formyl-CoA can be a HACS. In certain aspects, the HACS is selected from those in Table A. In certain aspects, the HACS is RuHACS. In yet further aspects, the HACS is ApbHACS, CfhHACS, RcbHACS, PspHACS, AcHACS, DhcHACS. In certain specific aspects the HACS is ApbHACS. In yet further aspects, the HACS is ApbHACS, DhcHACS, RcbHACS, PdsHACS and PspHACS. In yet further aspects, the HACS is a 2-hydroxyacyl-CoA lyase is selected from Homo sapiens hacl1, Rattus norvegicus hacl1, Dictyostelium discoideum hacl1, and Mus musculus hacl1, and wherein the DNA molecule encoding the oxalyl-CoA decarboxylase is selected from Mycobacterium sp. MOTT36Y W7S_25140, Mycobacterium tuberculosis TKK-01-0051 K875_00487, Polynucleobacter necessarius subsp. asymbioticus QLW-PIDMWA-1 Pnuc_0637, and E. coli oxc.

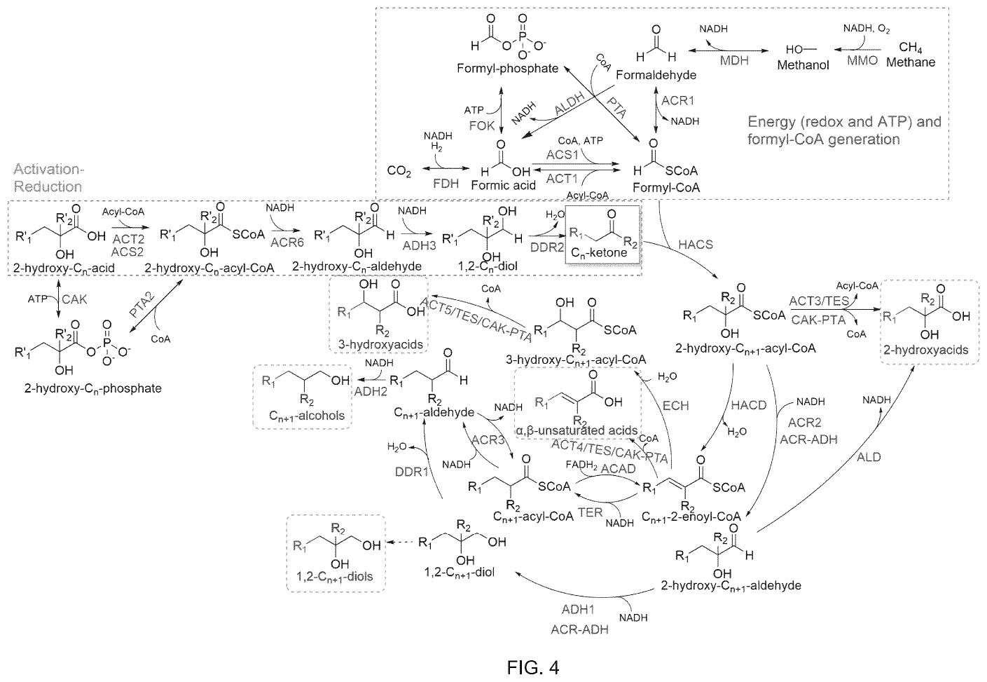

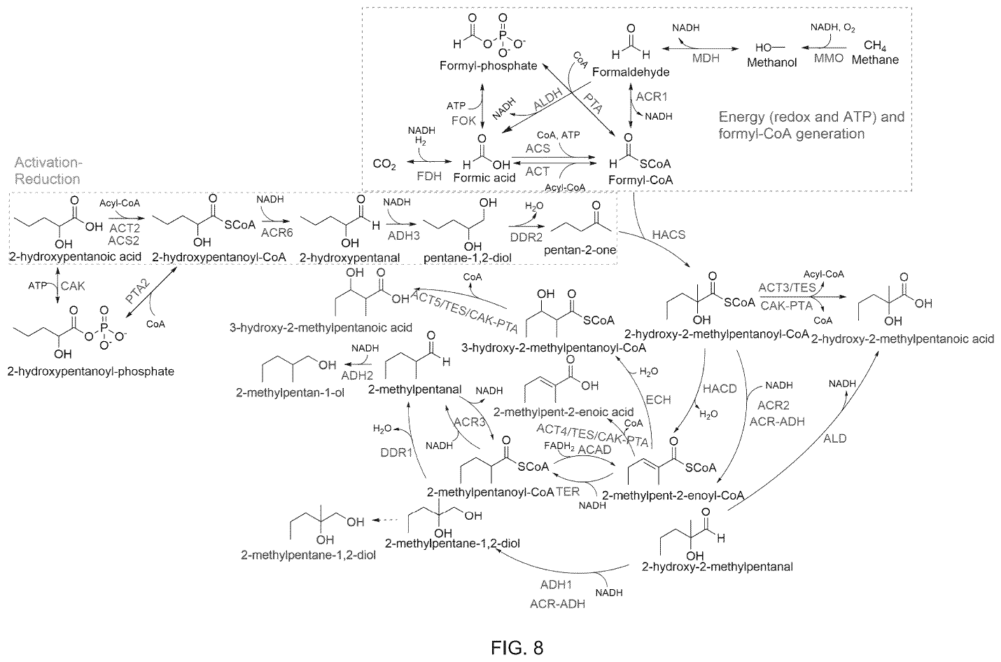

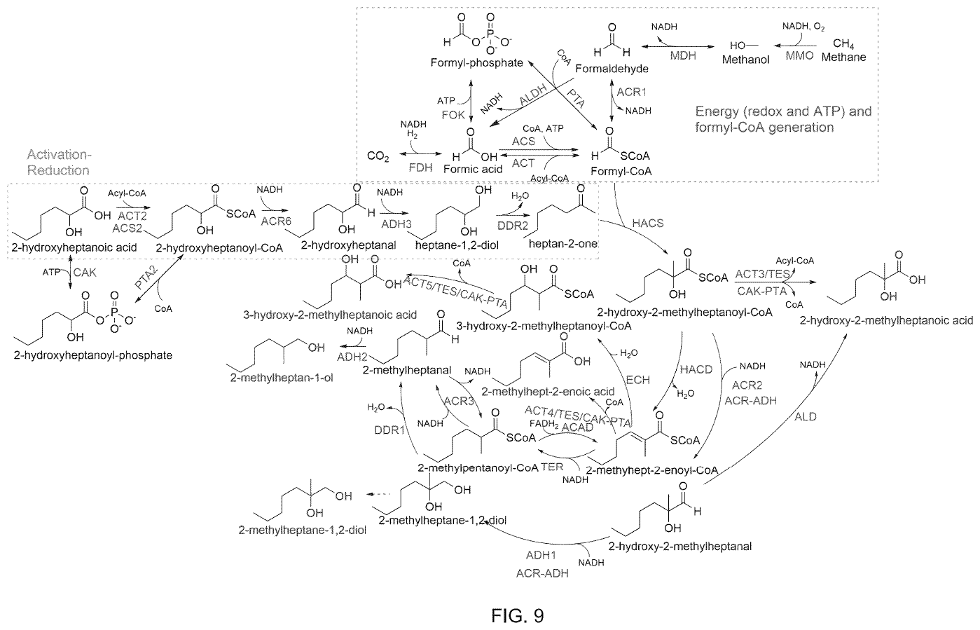

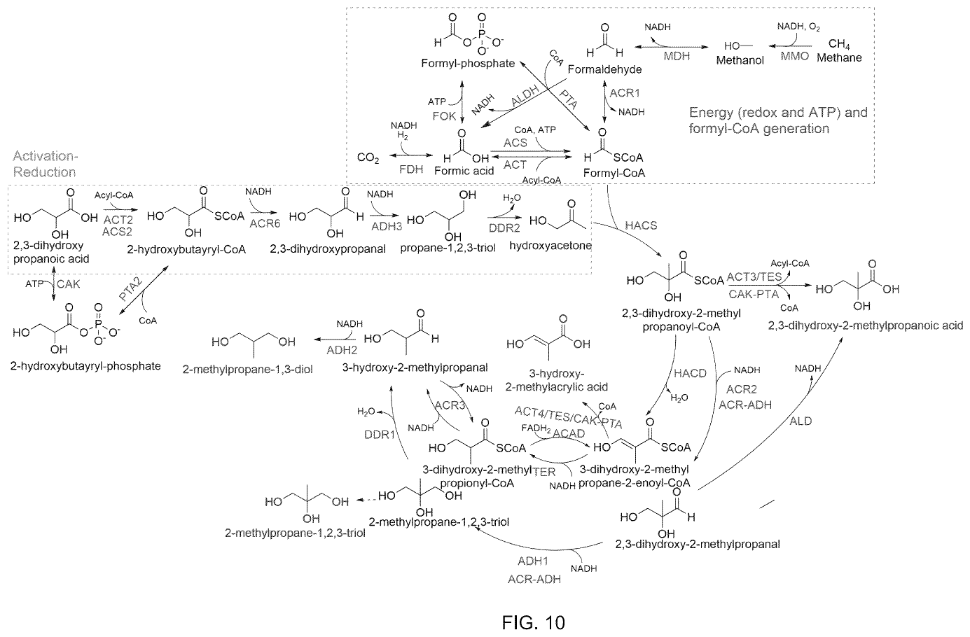

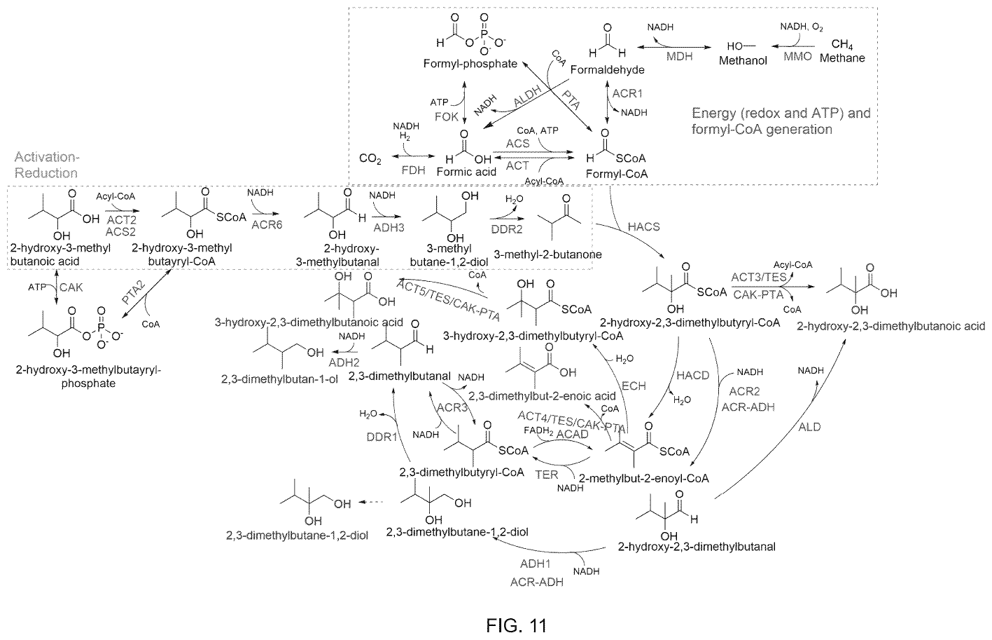

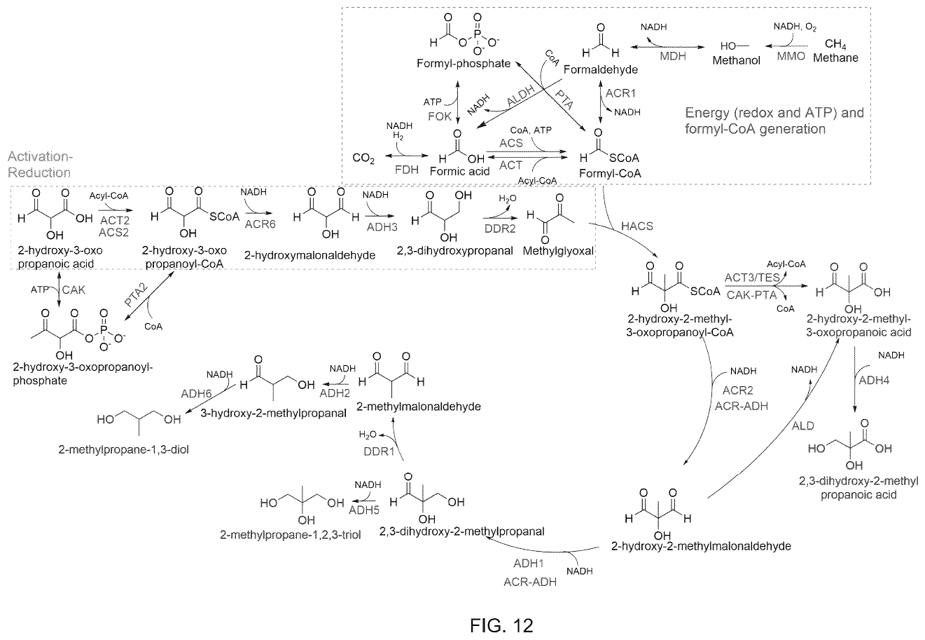

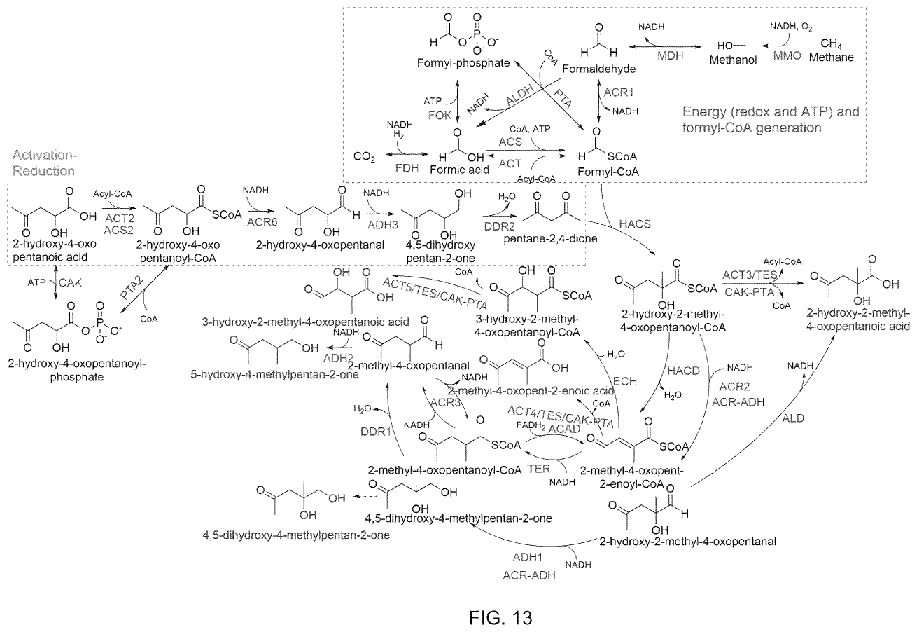

The branched 2-hydroxyacyl-CoA can be converted to a branched chain product using a pathway described herein. The pathway is exemplified by FIGS. 4, 6A, 7A, 8 to 13. The branched chain products include 2-hydroxyacids, 3-hydroxyacids, diols, alcohol, and ab-unsaturated acids.

In certain aspects, the branched product is a 2-hydroxyacid and the one or more enzymes that catalyze the conversion the 2-hydroxyacyl-CoA to the 2-hydroxyacid product comprises:
i. a thioesterase (TES),
ii. an acyl-CoA transferase (ACT), or
iii. a phosphotransacylase (PTA) and a carboxylate kinase (CAK).

In certain additional aspects, the branched product is 2-hydroxyacid and the one or more enzymes that catalyzes the conversion of the branched 2-hydroxyacyl-CoA to the product comprises:
i. an acyl-CoA reductase for catalyzing the conversion of the said 2-hydroxyacyl-CoA to a 2-hydroxyaldehyde; and
ii. an ALD enzyme for converting the 2-hydroxyaldehyde to the 2-hydroxyacid.

In certain specific aspects:
i. the ketone is acetone and the 2-hydroxyacid is 2-hydroxyisobutyric acid;
ii. the ketone is butan-2-one and the 2-hydroxyacid is 2-hydroxy-2-methylbutanoic acid;
iii. the ketone is pentan-2-one and the 2-hydroxyacid is 2-hydroxy-2-methylpentanoic acid;
iv. the ketone is hexan-2-one and the 2-hydroxyacid is 2-hydroxy-2-methylhexanoic acid;
v. the ketone is heptan-2-one and the 2-hydroxyacid is 2-hydroxy-2-methylheptanoic acid;
vi. the ketone is 1-cyclohexylpropan-2-one and the 2-hydroxyacid is 3-cyclohexyl-2-hydroxy-2-methylpropanoic acid;
vii. the ketone is acetophenone and the 2-hydroxyacid is 2-hydroxy-2-phenylpropanoic acid;
viii. the ketone is 1-phenylpropan-2-one and the 2-hydroxyacid is 2-hydroxy-2-methyl-3-phenylpropanoic acid;
ix. the ketone is 4-phenylpropan-2-one and the 2-hydroxyacid is 2-hydroxy-2-methyl-4-phenylbutanoic acid;
x. the ketone is hydroxyacetone and the 2-hydroxyacid is 2,3-dihydroxy-2-methylpropanoic acid
xi. the ketone is 3-hydroxy-3-methylbutan-2-one and the 2-hydroxyacid is 2,3-dihydroxy-2,3-dimethylbutanoic acid;
xii. the ketone is 2-oxopropanal and the 2-hydroxyacid is 2-hydroxy-2-methyl-3-oxopropanoic acid;
xiii. the ketone is pyruvic acid and the 2-hydroxyacid is 2-hydroxy-2-methylmalonic acid;
xiv. the ketone is acetoacetic acid and the 2-hydroxyacid is 2-hydroxy-2-methylsuccinic acid;
xv. the ketone is levulinic acid and the 2-hydroxyacid is 2-hydroxy-2-methylpentanedioic acid;
xvi. the ketone is cyclopropanone and the 2-hydroxyacid is 1-hydroxycyclopropane-1-carboxylic acid;
xvii. the ketone is cyclobutanone and the 2-hydroxyacid is 1-hydroxycyclobutane-1-carboxylic acid;
xviii. the ketone is cyclopentanone and the 2-hydroxyacid is 1-hydroxycyclopentane-1-carboxylic acid;
xix. the ketone is cyclohexanone and the 2-hydroxyacid is 1-hydroxycyclohexane-1-carboxylic acid;
xx. the ketone is pentan-3-one and the 2-hydroxyacid is 2-ethyl-2-hydroxybutanoic acid;
xxi. the ketone is hexan-3-one and the 2-hydroxyacid is 2-ethyl-2-hydroxypentanoic acid;
xxii. the ketone is heptan-3-one and the 2-hydroxyacid is 2-ethyl-2-hydroxyhexanoic acid;
xxiii. the ketone is heptan-4-one and the 2-hydroxyacid is 2-hydroxy-2-propylpentanoic acid;
xxiv. the ketone is diisobutyl ketone and the 2-hydroxyacid is 2-hydroxy-2-isobutyl-4-methylpentanoic acid;
xxv. the ketone is oxaloacetate and the 2-hydroxyacid is 1-hydroxyethane-1,1,2-tricarboxylic acid;
xxvi. the ketone is a-ketoglutaric acid and the 2-hydroxyacid is 1-hydroxypropane-1,1,3-tricarboxylic acid;
xxvii. the ketone is 3-(2-hydroxyphenyl)-2-oxopropanoic acid and the 2-hydroxyacid is 2-hydroxy-2-(2-hydroxybenzyl)malonic acid;
xxviii. the ketone is benzophenone and the 2-hydroxyacid is 2-hydroxy-2,2-diphenylacetic acid;
xxix. the ketone is dicyclohexyl ketone and the 2-hydroxyacid is 2-dicylohexyl-2-hydroxacetic acid;
xxx. the ketone is oxiran-2-one and the 2-hydroxyacid is 2-hydroxyoxirane-2-carboxylic acid;
xxxi. the ketone is oxetan-2-one and the 2-hydroxyacid is 2-hydroxyoxetane-2-carboxylic acid;
xxxii. the ketone is dihydrofuran-2(3H)-one and the 2-hydroxyacid is 2-hydroxytetrahydrofuran-2-carboxylic acid;
xxxiii. the ketone is tetrahydrofuran-2H-pyran-2-one and the 2-hydroxyacid is 2-hydroxytetrahydro-2H-pyran-2-carboxylic acid;
xxxiv. the ketone is aziridin-2-one and the 2-hydroxyacid is 2-hydroxyaziridine-2-carboxylic acid;
xxxv. the ketone is azetdin-2-one and the 2-hydroxyacid is 2-hydroxyaziridine-2-carboxylic acid;
xxxvi. the ketone is pyrrolidin-2-one and the 2-hydroxyacid is 2-hydroxypyrrolidine-2-carboxylic acid;
xxxvii. the ketone is piperdin-2-one and the 2-hydroxyacid is 2-hydroxypiperidine-2-carboxylic acid;
xxxviii. the ketone is biacetyl and the 2-hydroxyacid is 2-hydroxy-2-methyl-3-oxobutanoic acid;
xxxix. the ketone is cyclohexane-1,4-dione and the 2-hydroxyacid is 1-hydroxy-4-oxocyclohexane-1-carboxylic acid;
xl. the ketone is dihydroxyacetone and the 2-hydroxyacid is 2,3-dihydroxy-2-(hydroxymethyl)propanoic acid;
xli. the ketone is d-erythrulose and the 2-hydroxyacid is 2,3,4-trihydroxy-2-(hydroxymethyl)butanoic acid;
xlii. the ketone is d-ribulose and the 2-hydroxyacid is 2,3,4,5-tetrahydroxy-2-(hydroxymethyl)pentanoic acid;
xliii. the ketone is d-xylulose and the 2-hydroxyacid is 2,3,4,5-tetrahydroxy-2-(hydroxymethyl)pentanoic acid;
xliv. the ketone is d-fructose and the 2-hydroxyacid is 2,3,4,5,6-pentahydroxy-2-(hydroxymethyl)hexanoic acid;
xlv. the ketone is d-sorbose and the 2-hydroxyacid is 2,3,4,5,6-pentahydroxy-2-(hydroxymethyl)hexanoic acid;
xlvi. the ketone is d-tagatose and the 2-hydroxyacid is 2,3,4,5,6-pentahydroxy-2-(hydroxymethyl)hexanoic acid;
xlvii. the ketone is d-psicose and the 2-hydroxyacid is 2,3,4,5,6-pentahydroxy-2-(hydroxymethyl)hexanoic acid;
xlviii. the ketone but-3-en-2-one and the 2-hydroxyacid is 2-hydroxy-3-methylbut-3-enoic acid; or
xlix. the ketone is 3-bromo-2-oxopropanoic acid and the 2-hydroxyacid is 2-(bromomethyl)-2-hydroxymalonic acid.

In yet further aspects, the ketone is one shown in FIG. 5 (first column) and the branched 2-hydroxyacid product is one shown in FIG. 5 (second column).

In yet a further aspect, the product is a 1,2-diol and the one or more enzymes that catalyze the conversion of the branched 2-hydroxyacyl-CoA to the product comprises:
i. an acyl-CoA reductase that catalyzes the conversion of said 2-hydroxyacyl-CoA to a 2-hydroxyaldehyde; and
ii. an alcohol dehydrogenase that catalyzes the conversion of said 2-hydroxyaldehyde to the 1,2-diol.

In certain examples, the ketone is acetone and the 1,2-diol is 2-methylpropane-1,2-diol; the ketone is butan-2-one and the 1,2-diol is 2-methylbutane-1,2-diol; the ketone is pentan-2-one and the 1,2-diol is 2-methylbutane-1,2-diol; the ketone is heptan-2-one and the 1,2-diol is 2-methylheptane-1,2-diol; the ketone is hydroxacetone and the 1,2-diol is 2-methylproane-1,2,3-triol; the ketone is 3-methyl-2-butanone and the 1,2-diol is 2,3-dimethylbutane-1,2-diol; the ketone is methylglyoxal and the 1,2-diol is 2-methylpropane-1,2,3-triol; the ketone is pentane-2,4-dione and the 1,2-diol is 4,5-dihydroxy-4-methylpentan-2-one. In yet further aspects, the ketone is one shown in FIG. 5 (first column) and the 1,2-diol product is one shown in FIG. 5 (sixth column).

In another aspect, the branched product is an ab-unsaturated acid and the one or more enzymes that catalyze the conversion of the branched 2-hydroxyacyl-CoA to the product comprises:
i. an acyl-CoA dehydratase (HACD) for dehydration of the 2-hydroxyacyl-CoA to a 2-enoyl-CoA; and
ii. an acyl-CoA transferase, a thioesterase (TES), or a phosphotransacylase (PTA) and a carboxylate kinase (CAK) for conversion of the 2-enoyl-CoA to the ab-unsaturated acid.

In certain examples, the ketone is acetone and the ab-unsaturated acid is methacrylic acid; the ketone is butan-2-one and the ab-unsaturated acid is 2-methylbut-2-enoic acid; the ketone is pentan-2-one and the ab-unsaturated acid is 2-methylpent-2-enoic acid; the ketone is heptan-2-one and the ab-unsaturated acid is 2-methylhept-2-enoic acid; the ketone is hydroxyacetone and the ab-unsaturated acid is 3-hydroxy-2-methacrylic acid; the ketone is 3-methyl-2-butanone and the ab-unsaturated acid is 2,3-dimethylbut-2-enoic acid; and the ketone is pentane-2,4-dione and the ab-unsaturated acid is 2-methyl-4-oxopent-2-enoic acid. In yet further aspects, the ketone is one shown in FIG. 5 (first column) and the ab-unsaturated acid product is one shown in FIG. 5 (fifth column).

In yet a further aspect, the branched product is a 3-hydroxyacid and the one or more enzymes that catalyze the conversion of the branched 2-hydroxyacyl-CoA to the product comprises:
i. an acyl-CoA dehydratase (HACD) for dehydration of the 2-hydroxyacyl-CoA to a 2-enoyl-CoA;
ii. an enoyl-CoA hydratase (ECH) for conversion of the 2-enoyl-CoA to a 3-hydroxy-acyl-CoA;
iii. an acyl-CoA transferase, a thioesterase, or a phosphotransacylase (PTA) and a carboxylate kinase (CAK) for conversion of the 3-hydroxy-acyl-CoA to the 3-hydroxyacid.

In certain examples, the ketone is acetone and the 3-hydroxyacid is 3-hydroxyisobutryic acid; the ketone is butan-2-one and the 3-hydroxyacid is 3-hydroxy-2-methylbutanoic acid; the ketone is pentan-2-one and the 3-hydroxyacid is 3-hydroxy-2-methylpentanoic acid; the ketone is heptan-2-one and the 3-hydroxyacid is 3-hydroxy-2-methylheptanoic acid; the ketone is 3-methyl-2-butanone and the 3-hydroxyacid is 3-hydroxy-2,3-dimethylbutanoic acid; and the ketone is pentane-2,4-dione and the 3-hydroxyacid is 3-hydroxy-2-methyl-4-oxopentanoic acid.

In yet further aspects, the ketone is one shown in FIG. 5 (first column) and the 3-hydroxyacid product is one shown in FIG. 5 (third column).

In yet an additional embodiment, the product is an alcohol and wherein the one or more enzymes that catalyze the conversion of the branched 2-hydroxyacyl-CoA to the product comprises:
i. an acyl-CoA reductase for catalyzing the conversion of the said 2-hydroxyacyl-CoA to a 2-hydroxyaldehyde;
ii. an alcohol dehydrogenase for catalyzing the conversion of the said 2-hydroxyaldehyde to the 1,2-diol;
iii. a diol dehydratase for catalyzing the conversion of the 1,2-diol to an aldehyde; and
iv. an alcohol dehydrogenase for catalyzing the conversion of the aldehyde to the alcohol.

In certain example, wherein the ketone is acetone and the alcohol is isobutanol; the ketone is butan-2-one and the alcohol is 2-methylbutan-1-ol; the ketone is pentan-2-one and the alcohol is 2-methylpentan-1-ol; the ketone is heptan-2-one and the alcohol is 2-methylheptan-1-ol; the ketone is hydroxacetone and the alcohol is 2-methyl-1,3-diol; the ketone is 3-methyl-2-butanone and the alcohol is 2,3-dimethylbutan-1-ol; wherein the ketone is methylglyoxal and the alcohol is 2-methylpropane-1,3-diol; or the ketone is pentane-2,4-dione and the alcohol is 5-hydroxy-4-methylpentan-2-one. In yet further aspects, the ketone is one shown in FIG. 5 (first column) and the alcohol product is one shown in FIG. 5 (seventh column).

In yet a further aspect, the product is an alcohol and wherein the one or more enzymes that catalyze the conversion of the branched 2-hydroxyacyl-CoA to the product comprises:
i. an acyl-CoA dehydratase (HACD) for dehydration of the 2-hydroxyacyl-CoA to a 2-enoyl-CoA;
ii. a trans-2-enoyl-CoA reductase (TER) for catalyzing the conversion of the 2-enoyl-CoA to the acyl-CoA;
iii. an acyl-CoA reductase for catalyzing the conversion of the for the acyl-CoA to an aldehyde; and
iv. an alcohol dehydrogenase for catalyzing the conversion of the aldehyde to the alcohol.

In certain examples, wherein the ketone is acetone and the alcohol is isobutanol; the ketone is butan-2-one and the alcohol is 2-methylbutan-1-ol; the ketone is pentan-2-one and the alcohol is 2-methylpentan-1-ol; the ketone is heptan-2-one and the alcohol is 2-methylheptan-1-ol; the ketone is hydroxacetone and the alcohol is 2-methyl-1,3-diol; the ketone is 3-methyl-2-butanone and the alcohol is 2,3-dimethylbutan-1-ol; wherein the ketone is methylglyoxal and the alcohol is 2-methylpropane-1,3-diol; or the ketone is pentane-2,4-dione and the alcohol is 5-hydroxy-4-methylpentan-2-one. In yet further aspects, the ketone is one shown in FIG. 5 (first column) and the alcohol product is one shown in FIG. 5 (seventh column).

In certain aspects, the formyl-CoA can be prepared from a 1-carbon substrate (also referred to herein as a “1-carbon precursor”), for example, formate, formaldehyde, methanol, methane and carbon dioxide (see, for example, FIGS. 4, 6A, 7A, 8 to 13), and the conversion of the 1-carbon substrate to the formyl-CoA is catalyzed by one or more enzymes. In certain aspects, the 1-carbon substate is methanol and the one or more enzymes that catalyze the conversion of methanol to the formyl-CoA comprises methanol dehydrogenase (MDH) for catalyzing the conversion of the methanol to formaldehyde and an acyl-CoA reductase (ACR) for converting the formaldehyde to the formyl-CoA. In another example, the 1-carbon substate is formaldehyde and the one or more enzymes that catalyzes the conversion of the formaldehyde to the formyl-CoA comprises an acyl-CoA reductase (ACR) for catalyzing the conversion of the formaldehyde to the formyl-CoA. In yet another aspect, the 1-carbon substrate is formate and the one or more enzymes that catalyze the conversion of the formate to the formyl-CoA comprises an acyl-CoA synthetase, an acyl-CoA transferase, or a carboxylate kinase and a phosphotransacylase; or a carboxylic acid reductase and an acyl-CoA reductase; or an aldehyde dehydrogenase and an acyl-CoA reductase catalyzing the conversion of the formate to the formyl-CoA. In yet another aspect, the 1-carbon substrate is methane and the one or more enzymes that catalyze the conversion of methane to formyl-CoA comprise methane monooxygenase (MMO) for catalyzing the conversion of methane to methanol, methanol dehydrogenase (MDH) for catalyzing the conversion of the methanol to formaldehyde and an acyl-CoA reductase (ACR) for converting the formaldehyde to the formyl-CoA.

In some embodiments, the 2-hydroxyacyl-CoA formed through the addition of formyl-CoA to the ketone is reduced to a 2-hydroxyaldehyde by an acyl-CoA reductase (ACR; E.C. 1.2.1.−, e.g. 1.2.1.10, 1.2.1.76, 1.2.1.84). Further reduction of the 2-hydroxyaldehyde to give a 1,2-diol is possible by a suitable 1,2-diol oxidoreductase (DOR; E.C. 1.1.1.77) or alcohol dehydrogenase (ADH; E.C. 1.1.1.71). Dehydration of the 1,2-diol can be catalyzed by the activity of diol dehydratase (DDR; E.C. 4.2.1.28) to give an aldehyde, which can be further reduced to an alcohol by an alcohol dehydrogenase (ADH; E.C. 1.1.1.71).

In yet further aspects, the ketone is prepared from a carboxylic acid intermediate as described, for example, in WO2023287814, the contents of which are expressly incorporated by reference. For example, the preparation of the ketone from a 2-hydroxy carboxylic acid can be catalyzed by:
a. one or more enzymes to catalyze the conversion of the carboxylic acid into the corresponding acyl-CoA;
b. one or more enzymes to catalyze the conversion of the acyl-CoA of the preceding step into a ketone.

In certain aspects, the one or more enzymes that catalyze the conversion of the carboxylic acid into the corresponding acyl-CoA comprises:
a. an acyl-CoA synthetase or an acyl-CoA transferase that catalyzes the conversion of the carboxylic acid to the acyl-CoA; or
b. a carboxylate kinase that catalyzes the conversion of the carboxylic acid to a phosphate intermediate and a phosphotransacylase that converts the phosphate intermediate to the acyl-CoA; or
c. a carboxylic acid reductase that catalyzes the conversion of the carboxylic acid to an aldehyde and an acyl-CoA reductase that convers the aldehyde to the acyl-CoA; or
d. an aldehyde dehydrogenase that catalyzes the conversion of the carboxylic acid to an aldehyde, and an acyl-CoA reductase that converts the aldehyde to the acyl-CoA.

In further embodiments, the one or more enzymes to convert the acyl-CoA into the ketone comprises:
a. an acyl-CoA reductase and an alcohol dehydrogenase that catalyzes the conversion of the acyl-CoA to a 1,2-diol; and
b. a diol dehydratase that catalyzes the conversion of the 1,2-diol into the ketone.

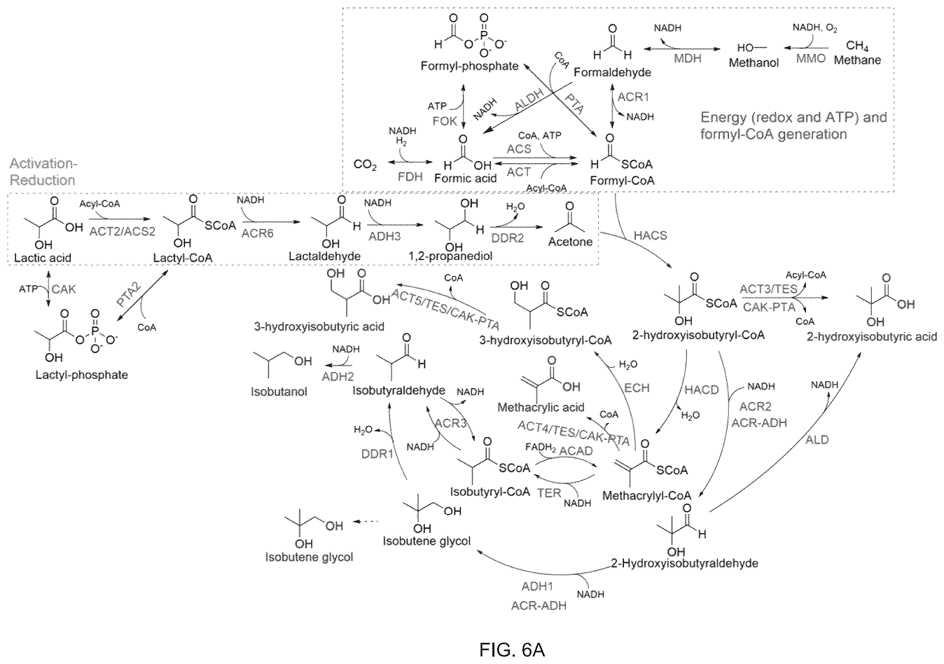

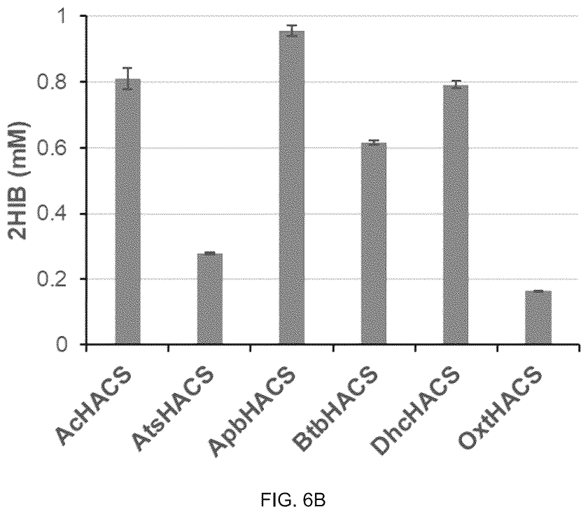

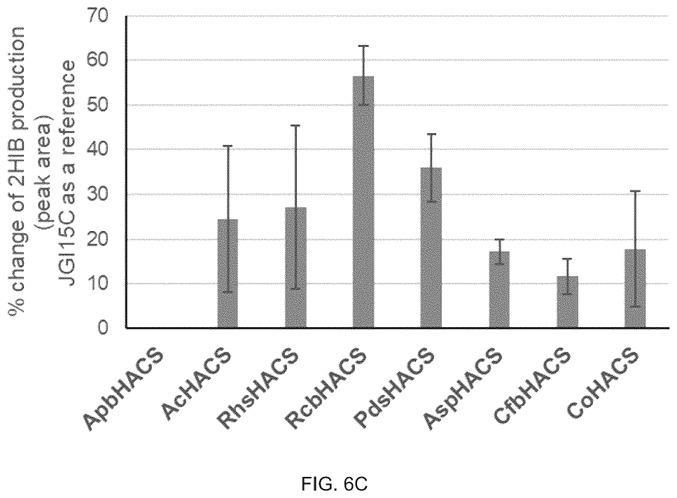

In one example, the carboxylic acid intermediate is lactic acid and is converted into the corresponding acyl-CoA, lactoyl-CoA (see for example, FIG. 6A). This step is referred to herein as CA activation. In general, CA activation involves the conversion of a carboxylic acid group to a CoA thioester and requires at least one of following steps: (1) the carboxylic acid is directly converted to the corresponding acyl-CoA by an acyl-CoA synthetase or an acyl-CoA transferase, (2) the carboxylic acid is converted to a phosphate intermediate by a carboxylate kinase which is then converted to the corresponding acyl-CoA by a phosphotransacylase, (3) the carboxylic acid is first reduced to the corresponding aldehyde by a carboxylic acid reductase which is then converted to the corresponding acyl-CoA by an acyl-CoA reductase, or (4) the carboxylic acid is first reduced to the corresponding aldehyde by an aldehyde dehydrogenase which is then converted to the corresponding acyl-CoA by an acyl-CoA reductase.

The second step in the disclosed systems and methods is the conversion of the acyl-CoA into a desired reduced product ketones. In general, diverse reduced products with varying functionality and chain length can be synthesized from the acyl-CoA intermediate.

For example, reduction of the acyl-CoA by an acyl-CoA reductase can generate an aldehyde (e.g., lactaldehyde). This aldehyde can be further reduced to an n-alcohol by an alcohol dehydrogenase and the alcohol can be converted to the ketone (e.g. acetone) by diol dehydratase (DDR). Alternatively, the aldehyde can serve as a precursor for formyl-CoA elongation using formyl-CoA as the C₁ building block in a reaction catalyzed by a 2-hydroxyacyl-CoA lyase (HACL) or an oxalyl-CoA decarboxylase (OXC). These enzymes can ligate formyl-CoA with a variety of carbonyl-containing acceptors of broad chain length and functionalization, including aldehydes and ketones. In some embodiments, the initial carboxylic acid and corresponding acyl-CoA contains a hydroxy (—OH) group at the second carbon (2-hydroxyacyl-CoA). In these situations, reduction of the CoA group by and an acyl-CoA reductase and an alcohol dehydrogenase results in the formation of a 1,2-diol which can be further dehydrated by a diol dehydratase to the corresponding ketone or aldehyde. This ketone or aldehyde can also serve as a precursor for formyl-CoA elongation using formyl-CoA as the C1 building block.

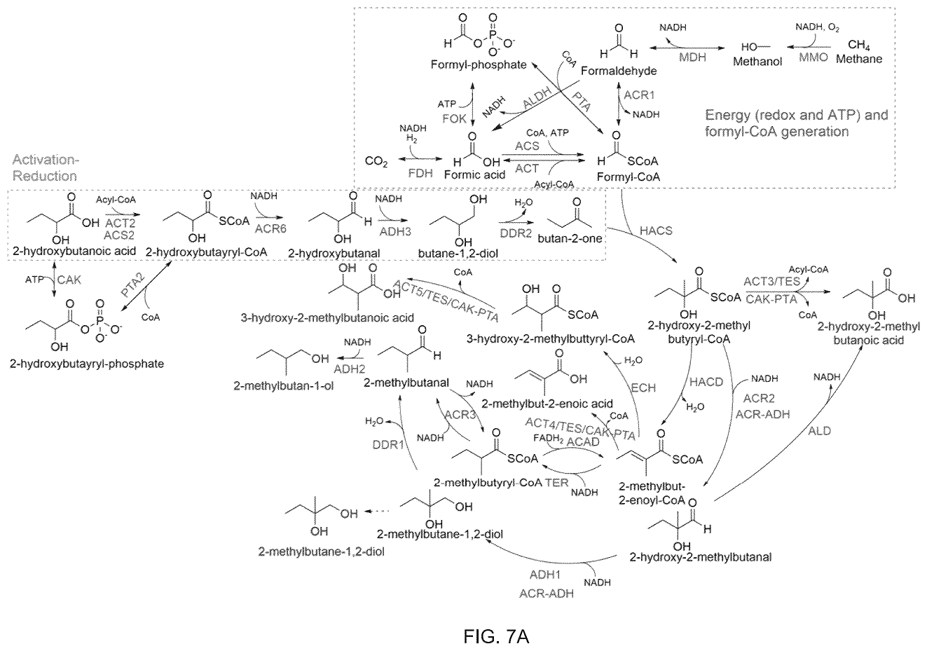

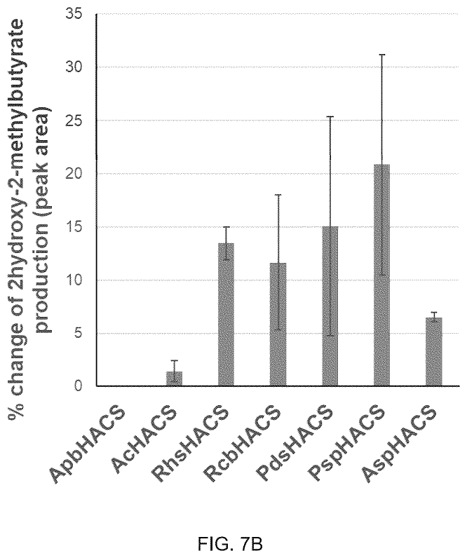

Additional examples demonstrating conversion of the carboxylic acid intermediate is 2-hydroxybutanoic acid, 2-hydroxypentanoic acid, 2-hydroxyheptanoic acid, 2,3-dihydroxy propanoic acid, 2-hydroxy-3-methyl butanoic acid, 2-hydroxy-3-oxo-propanoic acid, 2-hydroxy-4-oxo-pentanoic acid into the corresponding acyl-CoA and conversion of the acyl-CoA to a ketone (butan-2one, pentan-2-one, heptan-2-one, hydroxyacetone, 3-methyl-2-butanone, methylglyoxal, and pentane-2,4-dione, respectively are shown in FIGS. 7A, 8, 9, 10, 11, 12, and 13.

The third step in the disclosed systems and methods is the generation of reducing equivalents and ATP from an external energy source. This step is referred to herein as energy generation. In general, energy generation involves the oxidation of a supplied external energy source resulting in the generation of reducing equivalents and ATP. In some embodiments, reduced one-carbon (C1) molecules, such as methanol, are supplied as the energy source. In these situations, the C1 molecule is oxidized to CO2 by suitable enzymes with concomitant generation of NADH. For example, methanol can be oxidized to CO2 through a methanol dehydrogenase converting methanol to formaldehyde, a formaldehyde dehydrogenase converting formaldehyde to formate, and a formate dehydrogenase converting formate to CO2. In some embodiments, the reduced one-carbon (C1) is oxidized using suitable enzymes that generate both NAD(P)H and ATP. For example, methanol can be oxidized to CO2 through a methanol dehydrogenase converting methanol to formaldehyde, an acylating formaldehyde dehydrogenase converting formaldehyde to formyl-CoA, a phosphate formyltransferase converting formyl-CoA to formyl-phosphate, a formate kinase converting formyl-phosphate to formate, and a formate dehydrogenase converting formate to CO2. In some embodiments, hydrogen (H2) is supplied as the energy source. In these situations, H2 is converted to NAD(P)H through suitable NAD⁺-reducing hydrogenases.

In some embodiments, a combination of the above routes can be implemented at the same time such that for some molecules, products of the same chain length as the acyl-CoA are formed, whereas for other molecules, elongation takes place. Both routes can be simultaneously present at the same time in the same system.

In some embodiments, the described pathways are provided within the context of a microbial host. In some embodiments, the microbial host is cultured in a fermentation system to produce desired products. In other embodiments, a microbial system is used to produce the enzymes, which are then extracted from the microbes for use in a cell-free system. In other embodiments, the enzymes are produced separately and individually added to the system.

The pathway in a living system is generally made by transforming the microbe with one or more expression vector(s) containing a gene encoding one or more of the enzymes, but the genes can also be added to the chromosome by recombinant engineering, homologous recombination, gene editing, and similar techniques. Where the needed protein is endogenous, as is the case in some instances, it may suffice as is, but is usually overexpressed for better functionality and control over the level of active enzyme. In some embodiments, one or more, or all, such genes are under the control of an inducible promoter.

The enzymes can be added to the genome or via expression vectors, as desired. Preferably, multiple enzymes are expressed in one vector or multiple enzymes can be combined into one operon by adding the needed signals between coding regions. Further improvements can be had by overexpressing one or more, or even all of the enzymes, e.g., by adding extra copies to the cell via plasmid or other vector. Initial experiments may employ expression plasmids hosting 3 or more ORFs for convenience, but it may be preferred to insert operons or individual genes into the genome for stability reasons.

Still further improvements in yield can be had by reducing competing pathways, such as those pathways for making e.g., acetate, formate, ethanol, and lactate, and it is already well known in the art how to reduce or knockout these pathways. See e.g., U.S. Pat. Nos. 7,569,380, 7,262,046, 8,962,272, 8,795,991, 8,129,157, and 8,691,552, each incorporated by reference herein in its entirety for all purposes. Many others have worked in this area as well.

Following the construction of a suitable strain containing the engineered pathway, culturing of the developed strains can be performed to evaluate the effectiveness of the pathway at its intended goal. The organism can be cultured in a suitable growth medium, and can be evaluated for product formation from substrates. The amount of products produced by the organism can be measured by UPLC or GC, and indicators of performance such as growth rate, productivity, titer, yield, or carbon efficiency can be determined.

Further evaluation of the interaction of the pathway enzymes with each other and with the host system can allow for the optimization of pathway performance and minimization of deleterious effects. Because the pathway is under synthetic control, rather than under the organism's natively evolved regulatory mechanisms, the expression of the pathway is usually manually tuned to avoid potential issues that slow cell growth or production and to optimize production of desired compounds.

Additionally, an imbalance in relative enzyme activities might restrict overall carbon flux throughout the pathway, leading to suboptimal production rates and the buildup of pathway intermediates, which can inhibit pathway enzymes or be cytotoxic. Analysis of the cell cultures by HPLC or GC can reveal the metabolic intermediates produced by the constructed strains. This information can point to potential pathway issues.

As an alternative to the in vivo expression of the pathway, a cell free in vitro version of the pathway can be constructed. By purifying the relevant enzyme for each reaction step, the overall pathway can be assembled by combining the necessary enzymes in a reaction mixture. With the addition of the relevant cofactors and substrates, the pathway can be assessed for its performance independently of a host.

General methods for gene synthesis and DNA cloning, as well as vector and plasmid construction, are well known in the art, and are described in a number of publications. More specifically, techniques such as digestion and ligation-based cloning, as well as in vitro and in vivo recombination methods, can be used to assemble DNA fragments encoding a polypeptide that catalyzes a substrate to product conversion into a suitable vector. These methods include restriction digest cloning, sequence- and ligation-independent Cloning (SLIC), Golden Gate cloning, Gibson assembly, and the like. Some of these methods can be automated and miniaturized for high-throughput applications.

Gene cassettes for expressing an engineered metabolic pathway in a host microorganism are known in the art. The cassette can comprise one or more open reading frames (ORFs) which encode the enzymes of the introduced pathway, a promoter for directing transcription of the downstream ORF(s) within the operon, ribosome binding sites for directing translation of the mRNAs encoded by the individual ORF(s), and a transcriptional terminator sequence. Due to the modular nature of the various components of the expression cassette, one can create combinatorial permutations of these arrangements by substituting different components at one or more of the positions. One can also reverse the orientation of one or more of the ORFs to determine whether any of these alternate orientations improve the product yield.

In some embodiments, the host microorganism for expressing metabolic pathway genes contains plasmid vector(s) with the metabolic pathway expression cassettes mobilized into these organisms via conjugation.

In an alternative method for expressing metabolic pathway genes in a microbial host, the biosynthetic pathway genes can be inserted directly into the chromosome. Methods for chromosomal modification include both non-targeted and targeted deletions and insertions.

In some embodiments, the disclosed systems and methods also involve recovering and purifying the desired product from the fermentation broth. The method to be used depends on the physico-chemical properties of the product and the nature and composition of the fermentation medium and cells. For example, U.S. Pat. No. 8,101,808 describes methods for recovering C3-C6 alcohols from fermentation broth using continuous flash evaporation and phase separation processing. In some embodiments, solids may be removed from the fermentation medium by centrifugation, filtration, decantation. In some embodiments, the multi-carbon compounds are isolated from the fermentation medium using methods such as distillation, azeotropic distillation, liquid-liquid extraction, adsorption, gas stripping, membrane evaporation, or pervaporation.

Disclosed herein are several exemplary, non-limiting embodiments of the system, methods and compositions disclosed herein. In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

For example, in some embodiments, the microorganisms are modified to express one or more enzymes responsible for the catalytic reactions as depicted in the figures. Suitably, the microorganisms are modified to express two or more enzymes, three or more enzymes, four or more enzymes, five or more enzymes, and can be determined from the pathways depicted in the figures and not to be limited by the examples described herein. Further, one skilled in the art would be able to pick the combination of enzymes to be introduced into the microorganism to produce a genetically modified organism capable of modifying a carbon source to the desired chemical outcome.

In specific embodiments, microorganisms are modified with enzymes responsible the condensation of a ketone with formyl-CoA and/or conversion of the resulting 2-hydroxyacyl-CoA to a branched product using purified enzymes. Non-limiting examples of a HACS that can catalyze the condensation is ApbHACS, DhcHACS, RcbHACS, PdsHACS and PspHACS.

In some embodiments, microorganisms are modified with enzymes responsible for energy, redox and ATP, and formyl-CoA generation. In any and all combination, the microorganism is modified to include one or more of the following enzymes, including, for example, at least one of, mmoXyBZCD, pmoA1A2B1B2, BmMDH2, CnMCH2, BsMDH, LmACR, StEutE, CbAld, EcMhpF, PsDmpF, EcFrmA, PpFdhA, CcPta-Ack, EcACS, StACSstab, MhACS, ArACS, CaAbft, OfFrc, PsFdh and/or CbFdh.

In some embodiments, microorganisms are modified with enzymes responsible for carboxylic acid platform using formaldehyde as the intermediate aldehyde for the C1 elongation. In any and all combinations, the microorganism is modified to include one or more of the following enzymes, including, for example, at least one of, CcPta-A ck, EcPta-Ack, EcACS, StACSstab, MhACS, ArACS, CaAbfT, OfFrc, LmACE, RuHACL, BsmHACL, AcHACK, MeOXC4, LmACR, StPduP, EcAldA, EcfucO, KoPddABC, EcAdhE, CcPta-Ack, EcPta-Ack, EcYciA, EcGlcD, MtAld, BsAld, ALAT1, aldH1, dhaS, EcSerC, GOT1 and/or EcYdfG.

In some embodiments, microorganisms are modified with enzymes responsible for carboxylic acid platform using formamide as the intermediate aldehyde for the C₁ elongation. In any and all combinations, the microorganism is modified to include one or more of the following enzymes, including, for example, at least one of, MmFmdA, RuHACL, BsmHACL, AcHACL, MeOXC4, LmACR, EcAldA, EcfucO, KoPddA BC, EcAdhE, CcPta-Ack, EcPta-Ack, EcYciA, EcGlcD, MtAld, BsAld, ALA T1, aldH1, dhaS, EcSerC, GOT1, EcYdfG and/or EcGldA.

In some embodiments, microorganisms are modified to express enzymes responsible for energy (redox and ATP) and formyl-CoA generation pathways. In any and all combinations, the microorganism is modified to include one or more of the following enzymes, including, for example, at least one of, mmoB, mdh2, adh, frmA, LmACR, mhpF, dmpF, eutE, CcPta-AcK, EcPta-AcK, EcACS, StACSstabm MhACS, ArACS, abfT, frc and/or fdh.

In some embodiments, microorganisms are modified with enzymes responsible for activation-reduction and C1 elongation pathways. In any and all combinations, the microorganism is modified to include one or more of the following enzymes, including, for example, at least one of, ackA, tdcD, bukl, pta, ptb, yfaC, prpE, IvaE, AAE3, sucC, sucD, catl, scoC, cat3, yfdE, pct, eutE, pduP, adhE2, sucD, RuHACL, MeOXC4, JGI15, ydiF, tesA, ldh, ald, pdh, pduP, yahK, pduC, pduD, pduE, pddA, pddB, pddC, PiDD, yahk, lcdAB, acuN, gbuF, acuK, crt, lvaC, acuK, tesB, pcs, bcd, fade, pduP and/or ald.

In some embodiments, microorganisms are modified with enzymes responsible for carboxylic acid platform using methyl ketones for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using acetone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using butanone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using pentanone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using heptanone for the C-1 elongation pathway. In any and all combinations, these enzymes include at least one of the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using hydroxyacetone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using 3-methyl-2-butanone for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using methylgloxyal for the C-1 elongation pathway. In any and all combinations, the microorganism is modified to include one or more of the following enzymes, including, for example, at least one of, mmoXYBDCD, pmoA1A2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In some embodiments, microorganisms are modified with enzymes responsible for carboxylic acid platform using acetylacetone (pentane-2, 4-ddione) for the C-1 elongation pathway. In any and all combinations, the microorganism is modified to include one or more of the following enzymes, including, for example, at least one of, mmoXYBDCD, pmoAIA2B1B2, BmMDH2, CnMDH2, BsMDH, LmACR, StEutE, CbAld, EcMhpF, PsDmpF, pduP, AtAdhE, EcMhpF, CcPta-Ack, EcPta-Ack, EcACS, StACSstab, MhACS, arACS, RuHACS, BsmHACS, DbhACS, AcHACS, EcFrmA, PpFdhA, fucO, gldA, rhaZ, yahK, adhA, yjgB, yqhD, pduq, YLL056C, RiDD, pduCDE, pddABC, BpCaiD_2, CdHadBC, EcPaaZ, AtACX4, EgTER, CaCRT, PfECH, CaHbd, EcAldA, CaAbfT, OfFrc, PsFdh, CbFdh and/or EcYciA.

In specific embodiments, microorganisms are modified with enzymes responsible for the generation of formyl-CoA from formate. In any and all combinations, these enzymes include, for example, at least one of AbfT, CcPta-Ack, CaAbfT, OfFrc, EcACS, MhACS, MhACS3, ArACS, StACS6 and/or StACS.

In specific embodiments, microorganisms are modified with enzymes responsible for the generation of formyl-CoA from formaldehyde using acyl-CoA reductase. In any and all combinations, these enzymes include, for example, at least one of LmACR, StEutE, EcmhpF and/or PsDmpF.

In specific embodiments, microorganisms are modified with enzymes responsible for the generation of formyl-CoA from methanol derived formaldeyde using different methanol dehydrogenases in conjunction with LmACR. In any and all combinations, these enzymes include, for example, at least one of BmMDH, CnMdh, RuHACL and/or EcAldA.

In specific embodiments, microorganisms are modified with enzymes responsible for the conversion of formate to ethylene glycol. In any and all combinations, these enzymes include, for example, at least one of LmACR, OfFrc and/or HACS.

In specific embodiments, microorganisms are modified with enzymes responsible for the conversion of glycolate production. In any and all combinations, these enzymes include, for example, at least one of AbfT, HACS, RuHACL, BsmHACL, MeOXC4 and/or AcHACL.

Specific embodiments demonstrate the cell-free method for prototyping C1 elongation pathway for formaldehyde as a starting substrate, which could be generated through activation and reduction of carboxylic acid, formate. In any and all combination, the enzymes may include, LcACR, RuHACL, EcFucO, and/or KoPddABC.

In specific embodiments, microorganisms are modified with enzymes responsible for the synthesis of glycine from glycolate. In any and all combinations, these enzymes include, for example, at least one of AbfT, HACS, RuHACL, BsmHACL, MeOXC4 and/or AcHACL.

In specific embodiments, microorganisms are modified with enzymes responsible the implementation of the carboxylic acid (CA) platform using acetic acid (R═H) as the CA intermediate with C1 elongation pathways. In any and all combination, the enzymes may include, for example, JGI15, RuHACL and/orMeOXC4.

In specific embodiments, microorganisms are modified with enzymes responsible the implementation of the carboxylic acid (CA) platform using propionic acid (R═CH3) as the CA intermediate with C1 elongation pathways. In any and all combination, the enzymes may include, for example, JGI15, RuHACL and/or AcHACL.

In specific embodiments, microorganisms are modified with enzymes responsible the implementation of the carboxylic acid (CA) platform using glycolic acid (R═OH) as the CA intermediate with C1 elongation pathways. In any and all combination, the enzymes may include, for example, JGI15, RuHACL and/or AcHACL.

In specific embodiments, microorganisms are modified with enzymes responsible the implementation of the carboxylic acid (CA) platform using oxalic acid (R═OOH) as the CA intermediate with C1 elongation pathways. In any and all combination, the enzymes may include, for example, JGI15 and/or RuHACL.

In specific embodiments, microorganisms are modified with enzymes responsible the condensation of methyl ketones with formyl-CoA using purified enzymes. In any and all combination, the enzymes may include, for example, CaAbfT and/or BsmHACS.

In specific embodiments, microorganisms are modified with enzymes responsible the implementation of the condensation of methyl ketone with formyl-CoA (generated from formate) in vivo using growing cells, with acetone used as a representative methylketone. In any and all combination, the enzymes may include, for example, BsmHACS and/or AcHCS.

Expression of the desired enzymes from the constructed strain can be conducted in liquid culture, e.g., shaking flasks, bioreactors, chemostats, fermentation tanks and the like. Gene expression is typically induced by the addition of a suitable inducer, when the culture reaches an optical density of approximately 0.5-0.8. Induced cells can be grown for about 4-8 hours, at which point the cells can be pelleted and saved to −20° C. Expression of the desired protein can be confirmed by running cell pellet samples on SDS-PAGE.

The expressed enzyme can be directly assayed in crude cell lysates, simply by breaking the cells by chemical, enzymatic, heat or mechanical means. Depending on the expression level and activity of the enzyme, however, purification may be required to be able to measure enzyme activity over background levels. Purified enzymes can also allow for the in vitro assembly of the pathway, allowing for its controlled characterization. N-terminal or C-terminal HIS-tagged proteins can be purified using e.g., a Ni-NTA Spin Kit (Qiagen, Venlo, Limburg) following the manufacturer's protocol, or other methods could be used. The HIS-tag system was chosen for convenience only, and other tags are available for purification uses. Further, the proteins in the final assembled pathway need not be tagged if they are for in vivo use. Tagging was convenient, however, for the enzyme characterization work performed herein.

The reaction conditions for enzyme assays can vary with the type of enzyme to be tested. In general, however, enzyme assays follow a similar general protocol. Purified enzyme or crude lysate is added to a suitable reaction buffer. Reaction buffers typically contain salts, necessary enzyme cofactors, and are at the proper pH. Buffer compositions often change depending on the enzyme or reaction type. The reaction is initiated by the addition of substrate, and some aspect of the reaction related either to the consumption of a substrate or the production of a product is monitored.

Choice of the appropriate monitoring method depends on the compound to be measured. Spectrophotometric assays are convenient because they allow for the real time determination of enzyme activity by measuring the concentration dependent absorbance of a compound at a certain wavelength. There are not always compounds with a measurable absorbance at convenient wavelengths in the reaction, unfortunately. In these situations, other methods of chemical analysis may be necessary to determine the concentration of the involved compounds. Gas chromatography (GC) is convenient for the quantification of volatile substances, of which fatty acids and aldehydes are of particular relevance. Internal standards, typically one or more molecules of similar type not involved in the reaction, is added to the reaction mixture, and the reaction mixture is extracted with an organic solvent, such as hexane. Fatty acid samples, for example, can be dried under a stream of nitrogen and converted to their trimethylsilyl derivatives using BSTFA and pyridine in a 1:1 ratio. After 30 minutes of incubation, the samples are once again dried and resuspended in hexane to be applied to the GC. Samples can be run (e.g., on an Agilent 7890B Series Custom Gas Chromatography system equipped with a 5977B Inert Plus Mass Selective Detector Turbo EI Bundle and an Agilent HP-5-ms capillary column) (Agilent Technologies, CA).

Strain construction for the in vivo pathway operation will allow for the well-defined, controlled expression of the enzymes of the pathway. As before, E. coli or yeast is an exemplary host of choice for the in vivo pathway, but other hosts could be used. The Duet system (Novagen, Darmstadt, Germany), allows for the simultaneous expression of up to eight proteins by induction with IPTG in E. coli, and initial experiments used this host.

Pathway enzymes can also be inserted into the host chromosome, allowing for the maintenance of the pathway without requiring antibiotics to ensure the continued upkeep of plasmids. There are also, theoretically, an infinite number of genes that can be placed on the chromosome, as chromosomal expression does not require separate origins of replication as is the case with plasmid expression.

DNA constructs for chromosomal integration usually include an antibiotic resistance marker with flanking FRT sites for removal, a well characterized promoter, a ribosome binding site, the gene of interest, and a transcriptional terminator. The overall product is a linear DNA fragment with 50 base pairs of homology for the target site on the chromosome flanking each side of the construct.

However, the Flp-FRT recombination method is only one example of adding genes to a chromosome, and other systems are available, such as the RecBCD pathway, the RecF pathway, RecA recombinase, non-homologous end joining (NHEJ), Cre-Lox recombination, TYR recombinases and integrases, SER resolvases/invertases, SER integrases, PhiC31 Integrase, and the like. Chromosomal modifications in E. coli can also be achieved by the method of recombineering, as known to those skilled in the art.

In a recombineering method, for example, the cells are prepared for electroporation following standard techniques, and the cells transformed with linear DNA that contains flanking 50 base pair targeting homology for the desired modification site. For seamless integration of a DNA construct, a two-step approach can be taken using a cassette that contains both positive and negative selection markers, such as the combination of cat and sacB. In the first round of recombineering, the cat-sacB cassette with targeting homology for the desired modification site is introduced to the cells. The cat gene provides resistance to chloramphenicol, which allows for positive recombinants to be selected for on solid media containing chloramphenicol. A positive isolate can be subjected to a second round of recombineering introducing the desired DNA construct with targeting homology for sites that correspond to the removal of the cat-sacB cassette. The sacB gene encodes for an enzyme that provides sensitivity to sucrose. Thus, growth on media containing sucrose allows for the selection of recombinants in which the cat-sacB construct was removed. P1 phage lysates can be made from isolates confirmed by PCR and sequencing. The lysates can be used to transduce the modification into desired strains, as described previously.

Engineered strains expressing the designed pathway can be cultured under the following or similar conditions. Overnight cultures started from a single colony can be used to inoculate flasks containing appropriate media. Cultures are grown for a set period of time, and the culture media analyzed. The conditions will be highly dependent on the specifications of the actual pathway and what exactly is to be tested. For example, the ability for the pathway to be used for autotrophic growth can be tested by the use of formate or formaldehyde as a substrate in MOPS minimal media, as described by Neidhardt et al., supplemented with appropriate antibiotics, and inducers. Mixotrophic growth can be characterized by the addition of both single carbon compounds and glucose or glycerol.

Analysis of culture media after fermentation provides insight into the performance of the engineered pathway. Quantification of longer chain fatty acid products can be analyzed by GC. Other metabolites, such as short chain organic acids and substrates such as glucose or glycerol can be analyzed by HPLC.

Following the construction of a suitable strain containing the engineered pathway, fermentations of the developed strains can be performed to evaluate the effectiveness of the pathway at its intended goal, the production of products from single carbon compounds. The organism can be evaluated for growth on a variety of single carbon substrates, from methane to CO₂ and H₂, either autotrophically or mixotrophically, with the inclusion of an additional carbon source. The products produced by the organism can be measured by HPLC or GC, and indicators of performance such as growth rate, productivity, titer, yield, or carbon efficiency can be determined.

Further evaluation of the interaction of the heterologously expressed pathway enzymes with each other and with the host system can allow for the optimization of pathway performance and minimization deleterious effects. Because the pathway is under synthetic control, rather than under the organism's natively evolved regulatory mechanisms, the expression of the pathway can be tuned to avoid potential issues that slow cell growth or production and to optimize production of desired compounds.

For example, one potential issue might be the excessive overexpression of protein, which could lead to depletion of resources for cellular growth and product formation or induce a stress response in the host organism. Additionally, an imbalance in relative enzyme activities might restrict overall carbon flux throughout the pathway, leading to suboptimal production rates and the buildup of pathway intermediates, which can inhibit pathway enzymes or be cytotoxic.

Analysis of the cell cultures by HPLC or GC can reveal the metabolic intermediates produced by the constructed strains. This information can point to potential pathway issues. Additionally, -omics techniques, such as microarray or 2D-PAGE can give information about gene expression or protein expression, respectively. Genome scale modeling allows for the identification of additional modifications to the host strain that might lead to improved performance. Deletion of competing pathways, for example, might increase carbon flux through the engineered pathway for product production.

As an alternative to the in vivo expression of the pathway, a cell free, in vitro, version of the pathway can be constructed. By purifying the relevant enzyme for each reaction step, the overall pathway can be assembled by combining the necessary enzymes. With the addition of the relevant cofactors and single carbon compounds, the pathway can be assessed for its performance independently of a host.

The following description of examples provides additional details, any one of which can be subject to patenting in combination with any other. The specification in its entirety is to be treated as providing a variety of details that can be used interchangeably with other details, and it would be of inordinate length if one were to list every possible combination of genes/vectors/enzymes/hosts.

The invention is illustrated by the following non-limiting examples.

### EXAMPLES

### Example 1: Branched Chain Compounds Production Using Ketone as a Substrate for Condensation with Formyl-CoA

This example demonstrates the implementation of branched chain compounds production using ketone as a substrate for condensation with formyl-CoA. Ketones can be supplemented as a co-substrate or generated from common carbon source, such as glucose and/or other biomass-derived sugars, glycerol, or acetate, etc (FIG. 1). Conversion of common carbon source to ketones have been demonstrated in numerous microorganisms. Ketone can be produced from 2-hydroxy acid as mentioned above or through fatty acids synthesis or b-oxidation pathway demonstrated in the literatures (Appl Environ Microbiol 78:70-80, 2012; Metab Eng 62:84-94, 2020). In this example, a new route for branched chain compounds production was demonstrated using bio-based ketone as a co-substrate to condense with formyl-CoA to obtain a 2-hydroxy acid with branched chain (FIG. 1), it can be further converted to a series of branched chain compounds with downstream modifications, including but not limited to 2-hydroxyacid, 3-hydroxyacid, alcohol, 1,2-diol and a,b-unsaturated acids (FIGS. 4-13).

### Example 2: Carboxylic Acid Platform Using 2-Hydroxyacids-Derived Ketones as Substrate for Condensation with Formyl-CoA

This Example demonstrates the implementation of the condensation of ketones with formyl-CoA using purified enzymes. The generation of formyl-CoA catalyzed by CoA transferase and condensation catalyzed by HACS, for example, as described in the examples below. Ketones can be produced from 2-hydroxy acids as described herein or through fatty acids synthesis and β-oxidation pathway demonstrated in literatures (Appl Environ Microbiol 78:70-80, 2012; Metab Eng 62:84-94, 2020).

The enzymes acyl-CoA transferase and HACS were overexpressed and purified as described above. In vitro purified enzyme reactions for condensation of methyl ketone and formyl-CoA was comprised of 100 mM KPi pH 6.9, 10 mM MgCl₂, 0.15 mM TPP, 2 mM acetyl-CoA, 1 μM ApbHACL, 2 μM CaAbfT, 20 mM formate and 50 mM tested methyl ketones. Reactions were incubated at 30° C. for 24 hours unless otherwise specified. For this analysis, samples containing acyl-CoAs were first treated with 1/20 of the reaction volume of 10 M NaOH solution was added to terminate the reactions. After 30 min hydrolysis, 1/20 of the reaction volume of 10 N H₂SO₄ was added to improve the efficiency of acid extraction. The resulting sample was extracted into 4 mL ethyl acetate by vigorous vortexing for 90 seconds. The organic phase was separated and evaporated to dryness under a stream of nitrogen. The residue was dissolved in 50 μL pyridine and 50 μL N,O-Bis(trimethylsilyl)trifluoroacetamide (BSTFA) and incubated at 60° C. for 15 minutes. Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 m film thickness, 30 m length). Samples were analyzed by GC (1 L injection with a 20:1 split ratio) using helium as the carrier gas at a flowrate of 1.5 mL/min and the following temperature profile: initial 90° C. for 3 min; ramp at 15° C./min to 170° C.; ramp at 20° C./min to 300° C. and hold for 8 min. The injector and detector temperature were 250° C. and 350° C., respectively.

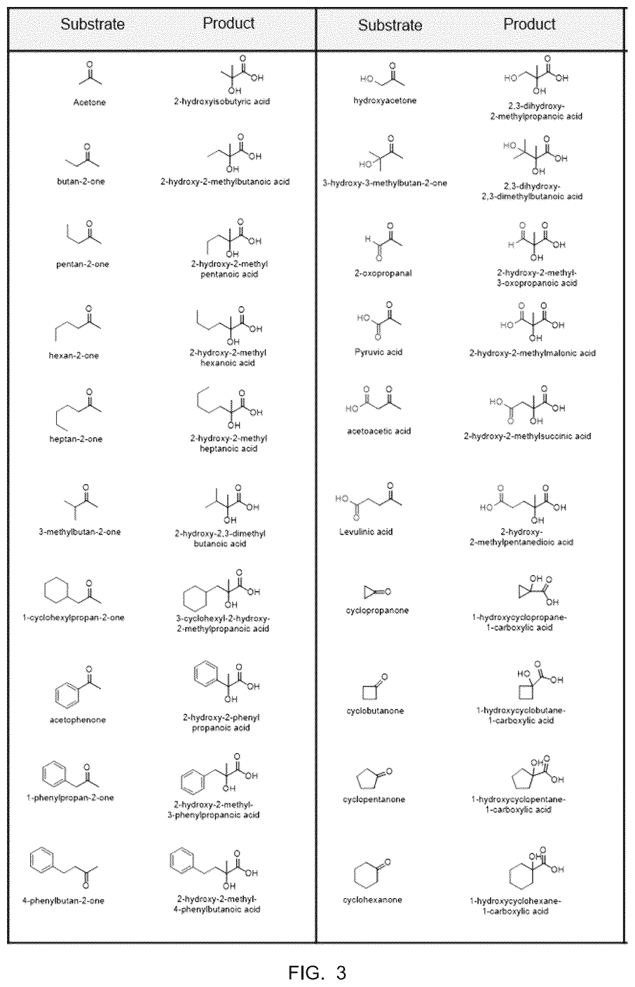

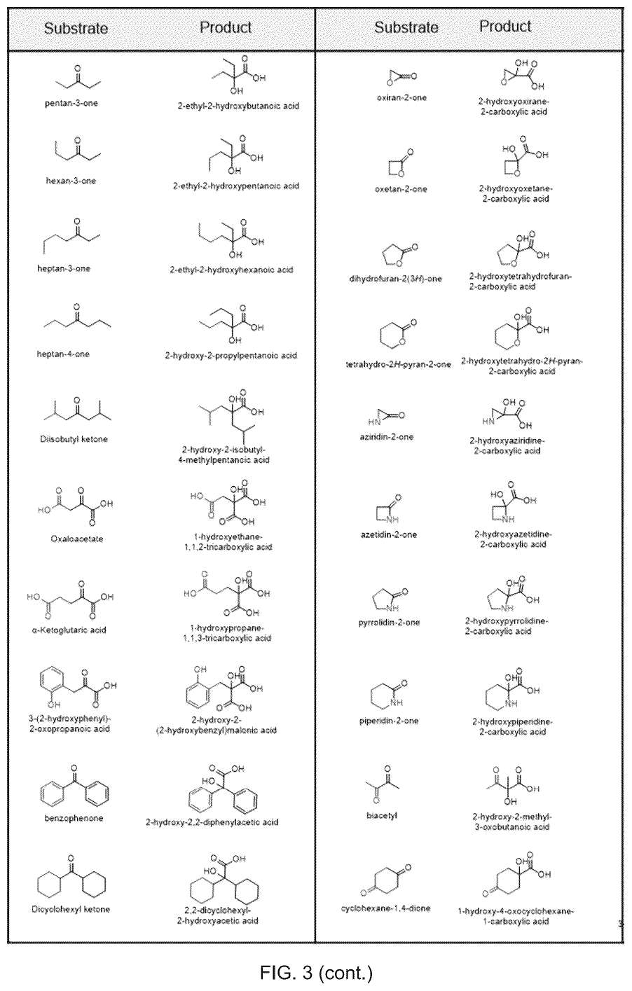

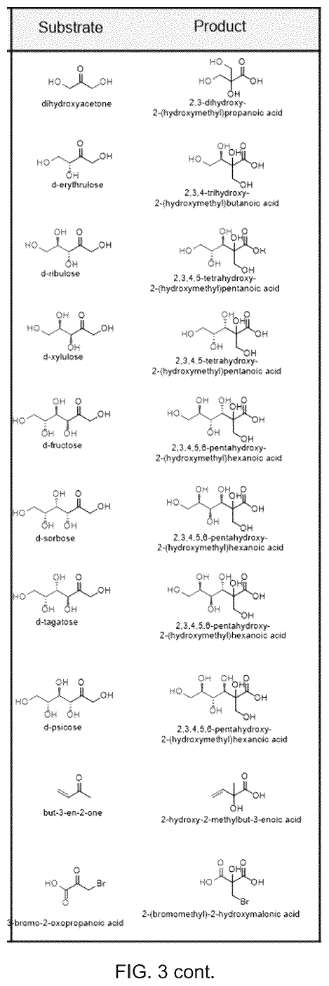

Ketones that are good carbonyl-containing substrate for acyloin condensation reactions with formyl-CoA include but are not limited to acetone, methyl ketone (Cₙ-ketone, n>3, butanone, pentanone and heptanone as examples), hydroxylated ketones (hydroxyacetone), and other functional ketones (acetylacetone, branched-chain ketones, methylglyoxal) etc. (FIG. 2). ApbHACS was able to catalyze the condensation of all tested ketones with formyl-CoA, as shown in FIG. 2, which indicates the other identified HACS is able to condensation of other ketones with formyl-CoA to produce 2-hydroxy-2 methyl acid and derivatives (FIG. 3 and Table 10). The ketones can be used for condensation including but not limited to acetone, methyl ketone (Cₙ-ketone, n>3, butanone, pentanone and heptanone as example), Hydroxylated ketones (hydroxyacetone), and other functional ketones (acetylacetone, branched-chain ketones, methylglyoxal) etc. (FIGS. 4-13 and Table 7).

### Example 3: Strategy Used to Identify Enzymes with Similar Structure and/or Function Based on Sequence Similarity

The purpose of this example is to provide an overview of workflow used to identify enzyme variants with desired activity starting from reference enzyme as query. In this example, 2-hydroxyacyl-CoA lyase, HACL from Rhodospirillales bacterium URHD0017 (RuHACL) is used as a starting query for identification of the first round 2-hydroxyacyl-CoA synthase (HACS) variants. Protein BLAST (pBLAST) is used with E-value cutoff based on the E-value between RuHACL and oxalyl-CoA decarboxylase, OXC from Escherichia coli (EcOXC) and Oxalobacter formigenes (OfOXC). To down select representative variants by clustering query results into families with similar sequences, we used CD-HIT web server (Huang et al. Bioinformatics, (2010). 26:680). More lenient restriction of 70% identity threshold was imposed for genes from prokaryotic origin whereas 50% was used for no taxonomic restriction. Clustering and picking representative genes using CD-HIT gave 93 HACS variants similar to RuHACL. Further curations from the list, including elimination of too long or too short sequences and variants from animalia, which is not likely to be expressed well in E. coli. As a result, we determined 34 remaining variants after the curations to be the initial round of HACS variants for synthesis and testing in E. coli as a host. The selected genes were then codon-optimized for expression in E. coli and synthesized in collaboration with Joint Genome Institute (JGI). 5 variants failed during synthesis, which gave 29 first round JGI HACS variants (JGI1 to JGI29) (TABLE 1).

The same strategy was used to identify second round HACS variants (JGIH) using JGI15 (ApbHACS), JGI19 (BtbHACS), JGI20 (DhcHACS) from first round variants and AcHACS as reference enzymes. Total 99 enzymes were identified that are closely related to AcHACL (AcHACS cluster), distantly related to AcHACS (distantly related to AcHACS cluster), JGI19 cluster, JGI15 cluster and JGI20 cluster. We have also identified 9 extra enzymes from I-TASSER (Yang et al. Nature Methods, 12: 7-8 (2015)) that are structurally similar to AcHACS, JGI15, JGI19 or JGI20 without considering the sequence similarity. Total 108 genes (JGIH1 to JGIH108) are codon-optimized and synthesized in collaboration with Joint Genome Institute and 99 variants are successfully constructed to pCDFDuet-1 expression vector for testing (TABLE 2).

### Example 4: Formaldehyde-Formyl-CoA Condensation In Vivo

The purpose of this example is to demonstrate the aspect of the invention pertaining to C1-C1 (formaldehyde-formyl-CoA) condensation for product synthesis in vivo.

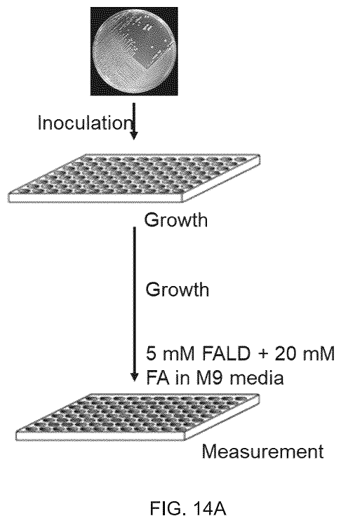

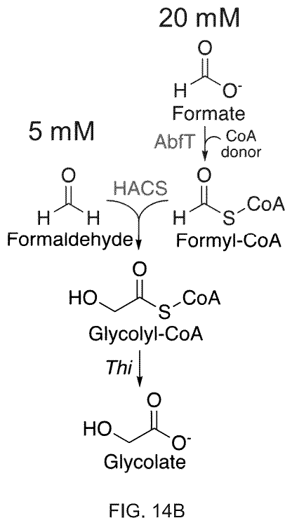

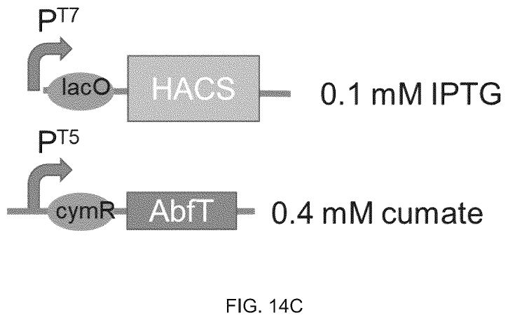

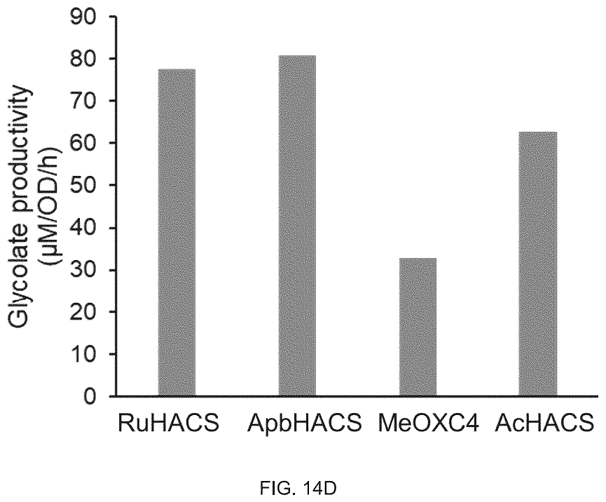

To implement the C1 elongation pathway for glycolate production in vivo, we engineered vectors to independently control expression of various HACS candidates and the acyl-CoA transferase from Clostridium aminobutyricum (CaAbfT), with HACS under control of the IPTG-inducible T7 promoter in pETDuet-1 and CaAbfT under control of a cumate-inducible T5 promoter in pCDFDuet-1 (FIG. 14C). As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD), which we expected could compete or interfere with the analysis of our pathway (FIG. 14B).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.5 mL of the above media further supplemented with 20 g/L glycerol, 10 g/L tryptone, and 5 g/L yeast extract. A single colony of the desired strain was cultivated overnight (14-16 hrs) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hrs post-inoculation.

Cells from the above pre-cultures were then centrifuged (4000 rpm, 22° C.), washed with the above minimal media without any carbon source, and resuspended with 1 mL of above minimal media containing indicated amounts of carbon source. 2.5 mM formaldehyde and 20 mM formate were added at 0 hr and were incubated at 30° C. and 1000 rpm in Digital Microplate Shaker (Fisher Scientific). After incubation at 30° C. for 0.5 hours, the cells were pelleted by centrifugation and the media analyzed (FIG. 14A).

The result indicates that CfhHACS is the best candidate under the given experimental conditions reaching up to 350 M/h per OD600.

### Example 5: Carboxylic Acid Platform Using Lactic Acid-Derived Acetone as Substrate for Condensation with Formyl-CoA Using Resting Cells

This example demonstrates screening of the HACS variants (first and second rounds) with various ketones as substrates for branched-chain compounds production. The HACS variants are tested using the high throughput screening platform as described in Example 4 by co-feeding 100 mM acetone and 20 mM formate with formate activation enzyme CaAbfT. The result shows that ApbHACS has the best performance in the first round HACS (FIG. 6B). RcbHACS has the best performance in the second round HACS with a 56% increase compared to ApbHACS for 2HIB production (FIG. 6C).

### Example 6: Carboxylic Acid Platform Using 2-Hydroxybutanoic Acid-Derived Butanone as Substrate for Condensation with Formyl-CoA Using Resting Cells

This example demonstrates screening of the second round HACS variants with butanone as substrates for branched-chain compounds production. The HACS variants are tested using the high throughput screening platform as described in Example 4 by co-feeding 100 mM butanone and 20 mM formate with formate activation enzyme CaAbfT. PspHACS had the best performance with a 12% increase compared to ApbHACS under tested conditions. (FIG. 7B).

### Example 7: Production of Acetone from Glucose

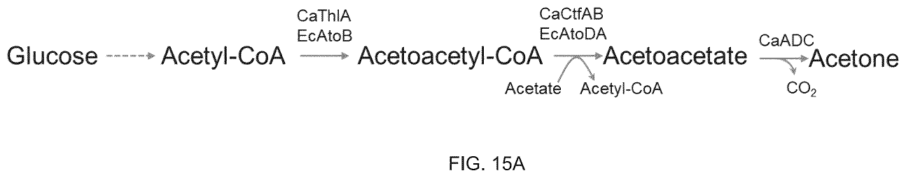

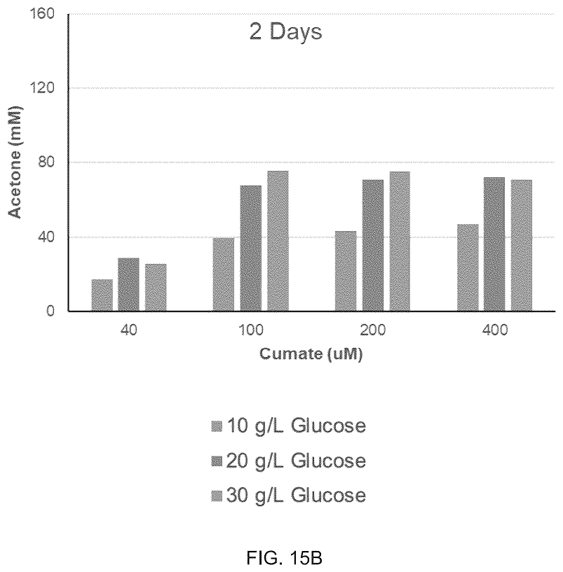

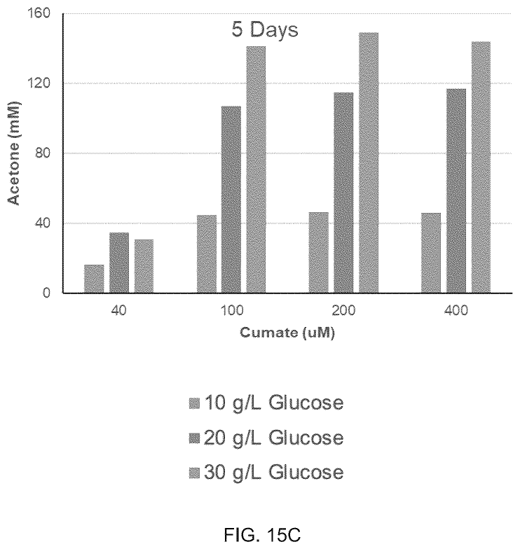

This example is to demonstrate the implementation of acetone production from glucose using growing cells. One glucose can generate two molecules of acetyl-CoA through glycolysis. Two Acetyl-CoA can be directly condensed to obtain acetoacetyl-CoA with the help of thiolase ThlA from Clostridium acetobutylicum or acetyl-CoA acetyltransferase AtoB from E. coli. Acetoacetyl-CoA is then converted to acetoacetate by CoA transferase (i.e. coenzyme A transferase CtfAB from C. acetobutylicum or acetyl-CoA: acetoacetyl-CoA transferase subunit a and β AtoDA from E. coli) and meanwhile activate acetate to acetyl-CoA. Acetone can be produced by decarboxylation of acetoacetate with acetoacetate decarboxylase ADC (FIG. 15A).

To implement the acetone production from glucose using growing cells, we constructed a plasmid pET-PCT5-CaThlA-CaADC-PCT5-EcAtoDA to overexpress CaThlA, CaADC and EcAtoDA using pET-PCT5 as the plasmid backbone. The plasmid was transformed into AC440 and in vivo production of acetone from glucose was conducted using M9-LB medium supplemented with glucose same as the method described above. 70 mM acetone was produced after 2 days fermentation and more than 100 mM acetone was obtained after 5 days fermentation when the cells were induced with more than 100 mM cumate (FIGS. 15B and 15C).

### Example 8: Production of Acetone from Acetate

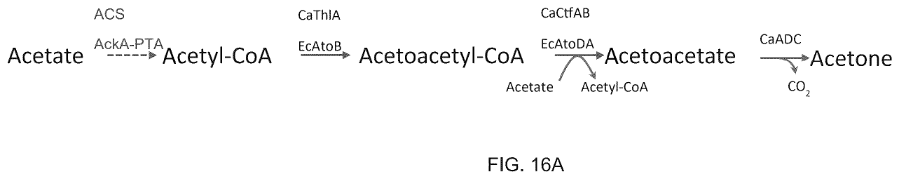

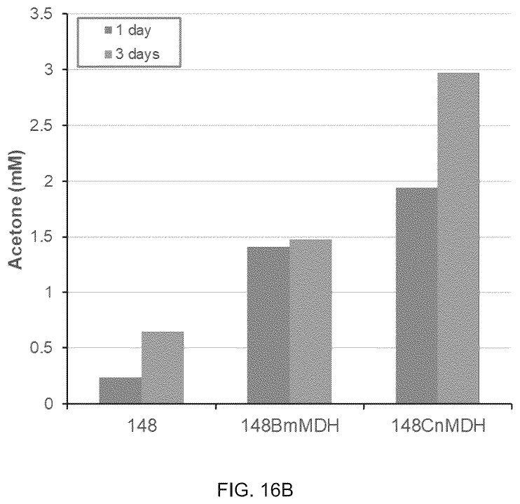

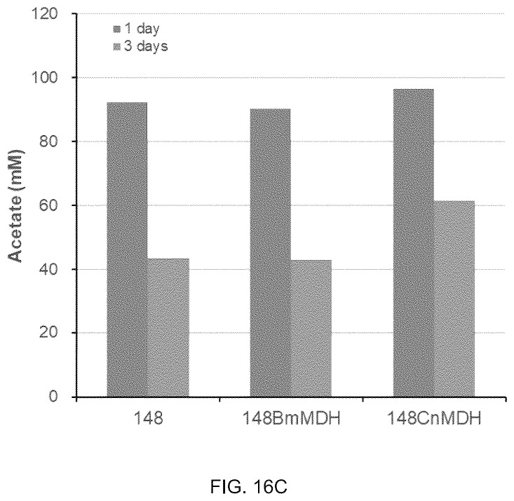

This example is to demonstrate the implementation of acetone production from acetate using growing cells. Acetate needs to be activated to acetyl-CoA firstly through acetyl-CoA synthetase ACS or kinase-phosphate acetyltransferase AckA-PTA. As described above, two acetyl-CoA can be converted to one acetone with the help of ThlA/AtoB, CtfAB/AtoDA, and ADC. During this conversion process, one acetate will be activated to acetyl-CoA by CftAB/AtoDA, so one extra acetate needs to be activated to acetyl-CoA per one acetone production (FIG. 16A). Acetone production in growing cells was observed, 0.65 mM acetone was accumulated after 3 days fermentation (FIG. 16B).

Both ACS and AckA-PTA routes need consume ATP (2 equivalents ATP for ACS and one for AckA-PTA route). To enhance the acetone production, methanol was co-fed to provide NADH which can be further used to generate ATP through Oxidative phosphorylation. To do so, methanol dehydrogenase from Bacillus methanolicus (BmMDH) and Cupriavidus necator (CnMDH) was co-overexpressed with the acetone producing module. Acetone production was enhanced to 1.4 mM and 3.0 mM even though equal or even less acetate was consumed (FIG. 16C).

### Example 9. Carboxylic Acid Platform Using Lactic Acid-Derived Acetone as Substrate for Condensation with Formyl-Coa Using Resting Cells

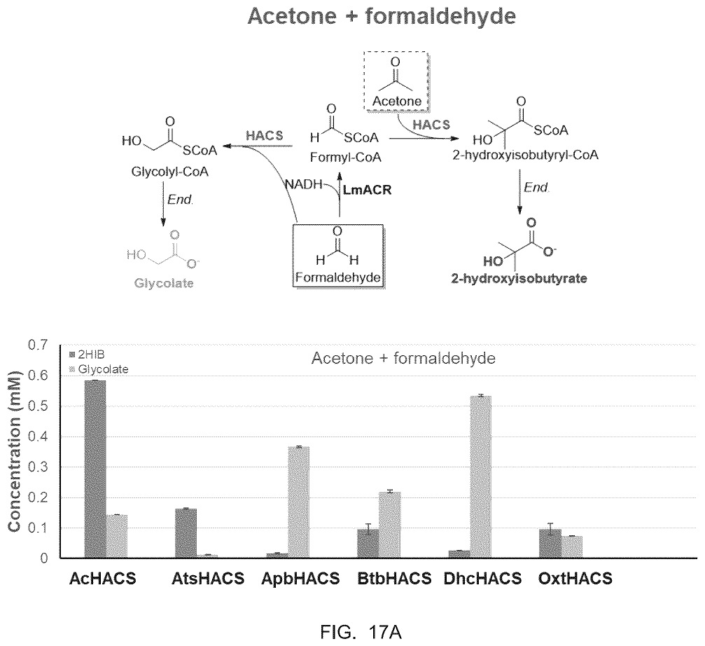

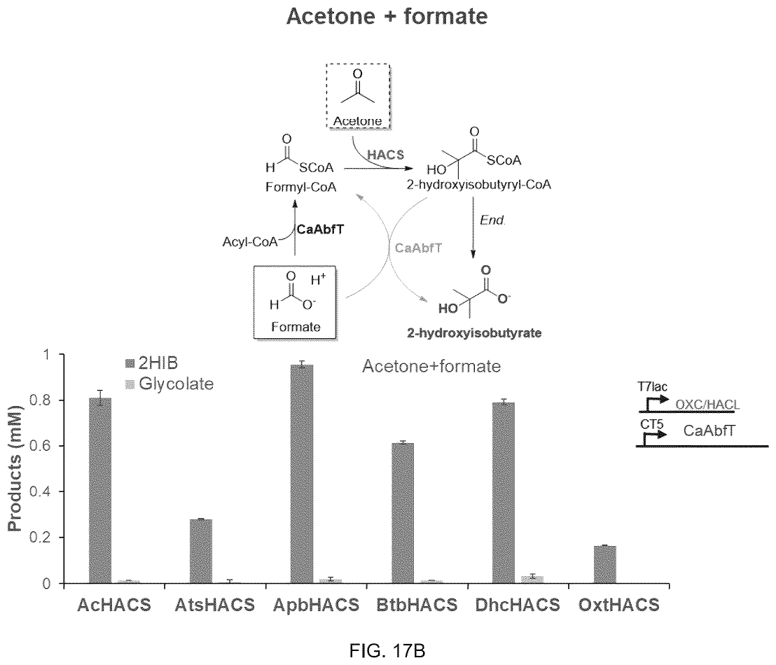

This example demonstrates screening of the first round HACS variants with ketones as co-substrate for branched-chain compounds production using acetone as an example for 2-hydroxyisobuteratic acid (2HIB). The HACS variants are tested using the high throughput screening platform as described in example 4 by co-feeding 100 mM acetone and 5 mM formaldehyde with acyl-CoA reductase (ACR) from Listeria monocytogenes. 2HIB was produced in some HACSs while AcHACS has the best performance. It is worth noting that glycolate was also observed and it is the main products in some HACSs, i.e. ApbHACS, DhcHACS (FIG. 17A). This is because these HACS prefer to use formaldehyde as a co-substrate to condense with formyl-CoA. To eliminate the crosstalk between formaldehyde and acetone, a new setting was excluded by overexpression of formate activation enzyme CaAbfT and using formate as a formyl-CoA source. Under this design, ApbHACS has the best performance, followed by AcHACS and DhcHACS (FIG. 17B).

### Example 10. Production of 2HIB from Acetone and One-Carbon Compounds Using Growing Cells

This example demonstrates the production of branched-chain compounds by condensation of ketone with formyl-CoA from one-carbon compounds (i.e. formate, (para)formaldehyde, and methanol) in vivo using growing cells, with acetone used as a representative ketone.

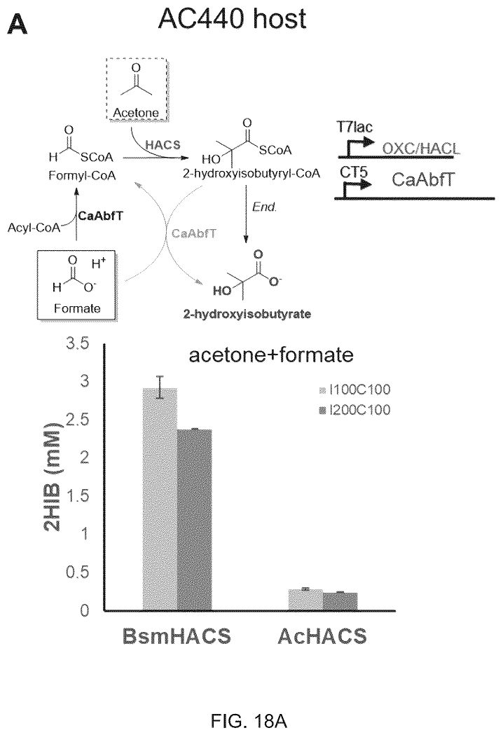

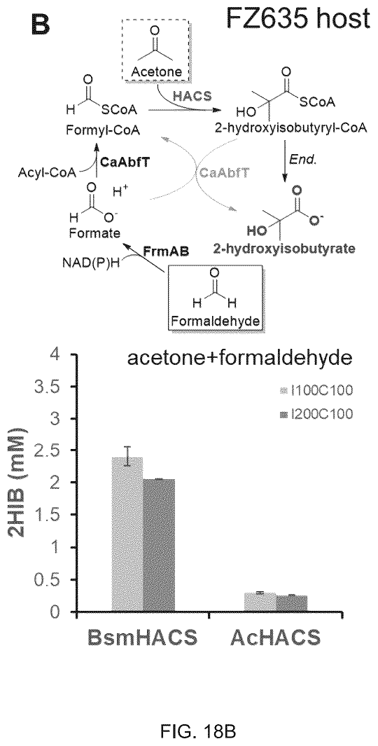

The HACS was first tested with acetone and formate, as it is shown in FIG. 18A, only two enzymes are needed, the formyl-CoA generation enzyme (FAE) and the condensation enzyme (HACS). ApbHACS was cloned under control of the IPTG-inducible T7 promoter in pCDFduet-1 and FAEs under control of a cumate-inducible T5 promoter in pETDuet-1. Meanwhile, an HACS variant recently identified in Actinomycetospora chiangmaiensis DSM 45062 (Frontiers in Microbiology 11:691, 2020) was also included and referred to as AcHACS.

The condensation of acetone with formyl-CoA from formate in actively growing cells was conducted using the M9-LB medium containing 6.78 g/L Na₂HPO₄, 3 g/L KH₂PO₄, 1 g/L NH₄Cl, 0.5 g/L NaCl, 2 mM MgSO₄, 100 μM CaCl₂), 15 μM thiamine-HCl, 10 g/L tryptone, and 5 g/L yeast extract additionally supplemented with the micronutrient solution of Neidhardt. A single colony of the desired strain was cultivated overnight (14-16 h) in LB medium with appropriate antibiotics and used as the inoculum (1%) to 50 mL centrifugation tubes containing 5 mL of M9-LB medium. Antibiotics (100 μg/mL carbenicillin, 100 g/mL spectinomycin) were included when appropriate. Cultures were then incubated at 30° C. and 250 rpm in a Lab Companion SI-600 rotary shaker (Jeio Tech, Seoul, South Korea) until an OD550 of ˜0.4 was reached, at which point appropriate amounts of inducer(s) (isopropyl β-D-1-thiogalactopyranoside and cumate) and substrates (acetone and formate) were added. Tubes were tightened and incubated for a total of 48 hr post-inoculation. The cells were pelleted by centrifugation and the media analyzed through HPLC or GC-MS as described above.

The growing cell experiment shows that both HACS catalyzed the condensation of acetone and formyl-CoA, but ApbHACS exhibited a better performance with up to 2.9 mM 2HIB produced after a 2-day fermentation when it was induced with 100 M IPTG and 100 M cumate (FIG. 18A).

The use of formaldehyde as a formate source was then tested. Formaldehyde can be converted to formate through the native detoxic system FrmAB, and then the formate can be used as described above. A new E. coli host FZ635 that deactivation of formate dehydrogenases (FdhF, FdnG and FdoG) was used for this experiment. Similar performance was obtained as compared to using acetone and formate (FIG. 18B). 2HIB production was further tested with acetone and methanol by co-overexpression of ApbHACS with BmMDH or CnMDH as mentioned above. In this design, we noticed that the MDH is not only able to convert methanol to formaldehyde but also produce significant amount of formate, so a theroesterase knockout strain CAL124 which has demonstrated to have better performance when using methanol as a co-substrate. As CnMDH is able to produce more formate from methanol, it has better performance on 2HIB production (FIG. 18C).

### Example 11: Production of 2HIB from Acetone and Formate Using Growing Cells in Bioreactor

This example demonstrates the implementation of 2HIB production from acetone and formate using growing cells in bioreactor. As in Example 10 described above, growing cells has a better performance on 2HIB production, a bioreactor run was excluded using the cells overexpressing HACS and CaAbfT.

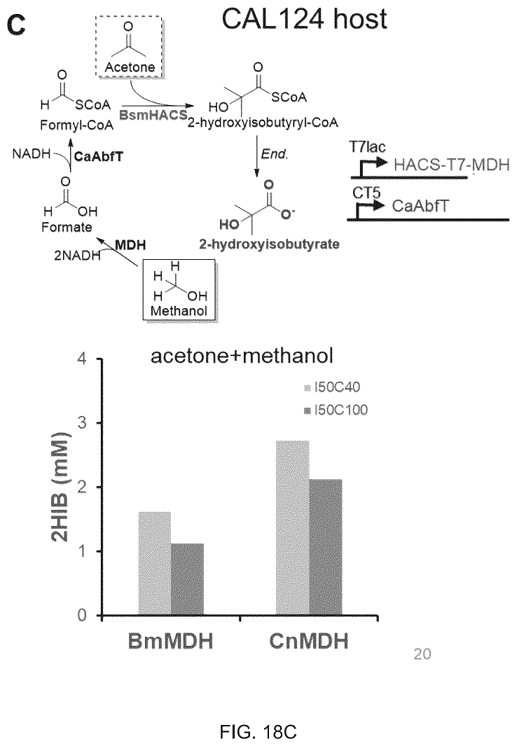

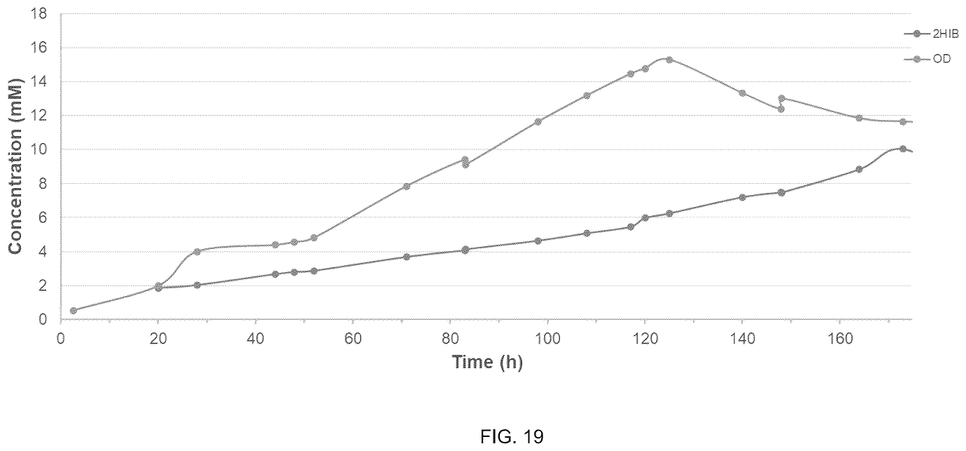

The condensation of acetone with formyl-CoA from formate in actively growing cells was conducted using the M9-LB medium containing 6.78 g/L Na₂HPO₄, 3 g/L KH₂PO₄, 1 g/L NH₄Cl, 0.5 g/L NaCl, 2 mM MgSO₄, 100 μM CaCl₂), 15 μM thiamine-HCl, 10 g/L tryptone, and 5 g/L yeast extract additionally supplemented with the micronutrient solution of Neidhardt68. A single colony of the desired strain was cultivated overnight (14-16 h) in LB medium with appropriate antibiotics and used as the inoculum (2%) to 750 mL bioreactor containing 500 mL of M9-LB medium. Antibiotics (100 μg/mL carbenicillin, 100 g/mL spectinomycin) were included when appropriate. Cells were then cultured at 30° C., 720 rpm with an airflow rate of 33 mL/min. cells were induced with 200 uM IPTG and 100 uM cumate after 2.5 h. 100 mM acetone and 20 mM formate was added right after induction. The pH was controlled at 7.0 by adding glucose (500 g/L stock solution), additional acetone was added when the acetone concentration is lower than 80 mM. 6 mM of 2HIB was accumulated after 5 days, and 10 mM 2HIB (1.04 g/L) was accumulated after 173 h (FIG. 19).

### Example 12: Branched Chain Compounds Production Through Condensation of Ketone and Formyl-Coa Using Acetone and Formate Derived Formyl-CoA as a Representative

This example demonstrates the branched-chain compounds production through condensation of ketone and formyl-CoA using purified enzymes; acetone was used as a representative ketone. The generation of formyl-CoA catalyzed by CoA transferase and condensation catalyzed by HACS is identical to the examples described above. Methyl ketones can be produced from 2-hydroxy acids as described above or through fatty acids synthesis and β-oxidation pathway demonstrated in literatures (Appl Environ Microbiol 78:70-80, 2012; Metab Eng 62:84-94, 2020).

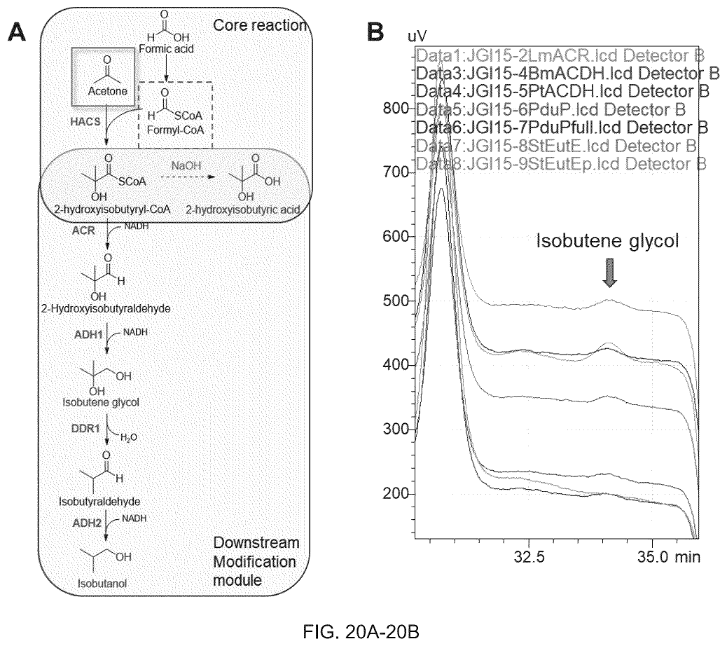

The enzymes acyl-CoA transferase CaAbfT, HACS ApbHACS, acyl-CoA reductase ACR (LmACR, BmACDH, PtACDH, RpPduP, StEutE), and NADH-dependent alcohol dehydrogenase FucO were overexpressed and purified as described above. In vitro purified enzyme reactions for condensation of acetone and formyl-CoA was comprised of 100 mM KPi pH 7.4, 10 mM MgCl₂, 0.15 mM TPP, 4 mM NADH, 2 mM acetyl-CoA, 1 μM ApbHACL, 2 μM CaAbfT, 50 mM formate and 200 mM acetone. The mixture was incubated at 30° C. for 3 min, and then 1 μM ACR and 1 μM FucO was added, and then the reactions were incubated at 30° C. for another 5 min unless otherwise specified. 2 μL of 10 M NaOH was added to 45 μL mixture for 30 min, followed by 3 μL 10 N H₂SO₄, and then applied for HPLC analysis as described above. Isobutene glycol production was observed, and StEutE has the best performance under tested conditions (FIG. 20).

### Example 13: Tolerance of 2HIB in Ac440

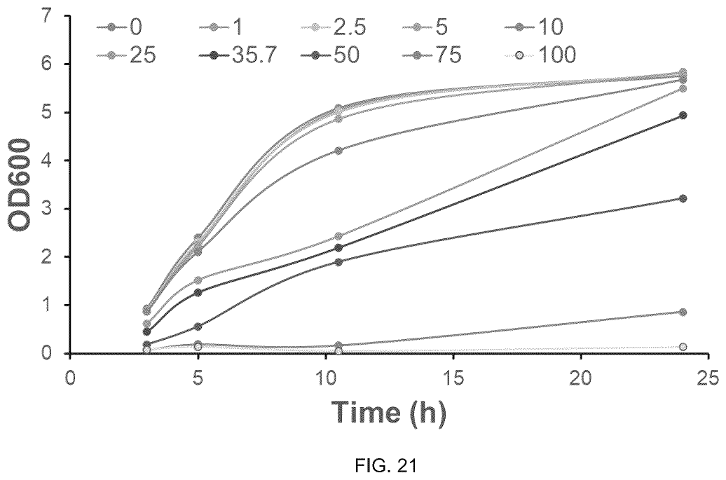

This example demonstrates the potential of AC440 as a host for 2HIB production by evaluating its tolerance ability on 2HIB. To check the tolerance of 2HIB, AC440 was grown in 5 mL of M9-LB supplemented with different concentration of 2HIB in 50 mL falcon tube, the cell density was monitored at 600 nm. The impact on growth could be observed at 25 g/L of 2HIB, while the AC440 is still able to grow and maintain its metabolic activity at the concentration of 2HIB at 75 g/L (FIG. 21), considering the cells will not suffer such high concentration of 2HIB at early stage, indicating AC440 is a good host for 2HIB production.

### Example 14: Overview of Enzymes Catalyzing Energy (Redox and ATP) and Formyl-CoA Generation

The purpose of this example is to provide exemplary genes, enzymes and pathways involved in the interconversion of one-carbon (C1) molecules to provide energy (in the form of NADH and ATP) and formyl-CoA required for C1 elongation reactions.

While providing exemplified embodiments of the invention, these are given as an illustration which combined with this disclosure allow a person skilled in the art to make additional modifications. The examples teach how to endow microorganisms with said capabilities, including specific pathway designs, required genes/enzymes to construct said pathways, methods for cloning and transformation, monitoring product formation and using the engineered microorganisms for production. While the examples show modified Escherichia coli strains, these modifications can easily be performed in other microorganisms of the same family Enterobacteriaceae as well as other bacterial species as well as yeast and fungi.

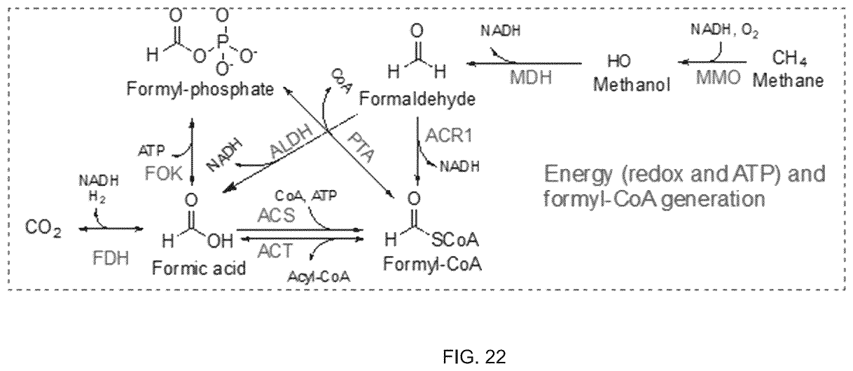

Methane is readily available C1 source from natural gas, landfills, and agriculture. Biological oxidation of methane to methanol is catalyzed by methane monooxygenases. Functional expression of soluble methane monooxygenase (sMMO) from Methylococcus capsulatus is demonstrated in E. coli as a host (bioRxiv 2021.08.05.455234) (FIG. 22 and Table 3). Subsequent oxidation of methanol to formaldehyde can be catalyzed by NAD⁺-dependent methanol dehydrogenases (MDH). MDH from Bacillus methanolicus MGA3 (BmMDH), Bacillus stearothermophilus (BsMDH) (Metab. Eng. 39:49-59, 2017) and Cupriavidus necator (Appl. Microbiol. Biotechnol. 100:4969-4983, 2016) are expressed and characterized in E. coli (FIG. 22 and Table 3). Formaldehyde can be directly oxidized to formic acid catalyzed by E. coli formaldehyde detoxification system (frmA). Alternatively, formaldehyde oxidation to formyl-CoA can be catalyzed by various acylating aldehyde dehydrogenase candidates (Nat. Chem. Biol. 15:900-906, 2019) (FIG. 22 and Table 3). Formyl-CoA can either be fed to the C₁ elongation platform or further converted to generate energy. Formyl-CoA hydrolysis to formate can be catalyzed by two different families of enzymes. Acyl-CoA transferases (ACT) can catalyze reversible CoA transfer from various CoA donor, such as glycolyl-CoA, acetyl-CoA or succinyl-CoA to formate. Phosphotransacylase-formate kinase (PTA-FOK) pair catalyze reversible phosphorylation of formyl-CoA to formyl-phosphate, followed by dephosphorylation to formate generating 1 ATP. While these two reactions are considered fully reversible, AMP-forming acyl-CoA synthetases are favored toward formate activation to formyl-CoA. Finally, formate can be further oxidized to CO₂ to generate NADH, catalyzed by soluble formate dehydrogenases, or the same enzyme can be used to reduce CO₂ to formate for formyl-CoA generation (FIG. 22 and Table 3).

### Example 15: Formyl-CoA Generation from Formic Acid

The purpose of this example is to demonstrate the aspect of the invention pertaining to generation of formyl-CoA from formate in vivo using both resting cells and growing cultures.

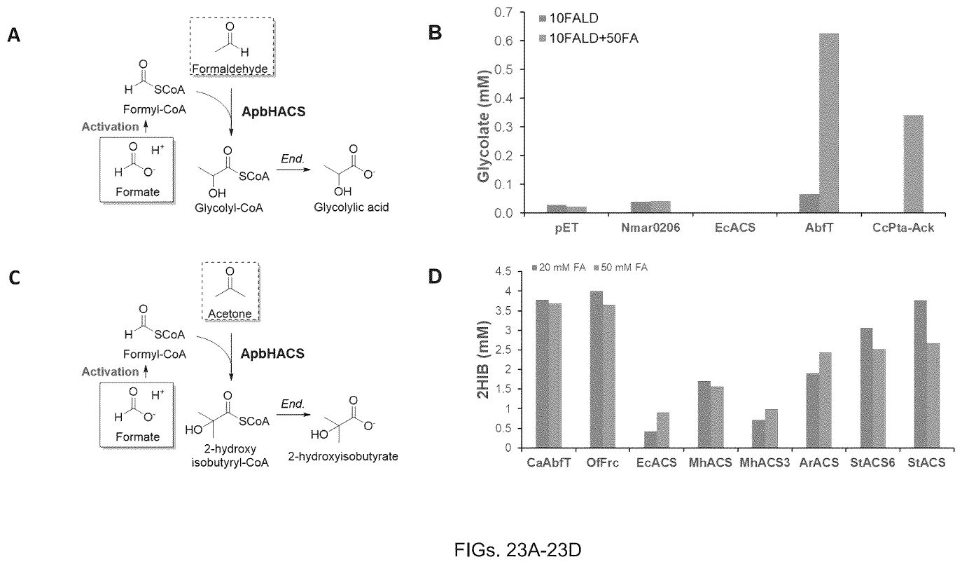

The formyl-CoA generated through formate activation enzymes (FAE) is further condensed with formaldehyde to produce glycolic acid. We engineered vectors to independently control expression of BsmHACS/ApbHACS (UniProt accession: A0A3C0TX30), sourced from beach sand metagenome, along with FAEs. BsmHACS/ApbHACS is under control of the IPTG-inducible T7 promoter in pCDFduet-1 and FAEs under control of a cumate-inducible T5 promoter in pETDuet-1 (FIG. 23A). FAEs were selected to represent the three major formate activation routes, including acyl-CoA synthase ACS (Nmar0206 from Nitrosopumilus maritimus and EcACS), acyl-CoA transferase ACT (AbfT from Clostridium aminobutyricum) and phosphotransacylase (PTA)-FOK (CcPta+CcAck from Clostridium cylindrosporum). As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD), which we expected could compete or interfere with the analysis of our pathway.

Cells from the above pre-cultures were then centrifuged (5000×g, 22° C.), washed twice with the above minimal media without any carbon source, and resuspended to an optical density ˜10. Five mL of this cell suspension and indicated amounts of carbon source (e.g. formaldehyde) was added to 25 mL Pyrex Erlenmeyer flasks (Corning Inc., Corning, NY) and sealed with foam plugs filling the necks. 10 mM formaldehyde and 50 mM formate were added at 0 hr. Flasks were incubated at 30° C. and 200 rpm in an NBS 124 Benchtop Incubator Shaker (New Brunswick Scientific Co., Inc., Edison, NJ). After incubation at 30° C. for 24 hours, the cells were centrifuged at 20817×g for 15 minutes and the supernatant analyzed by HPLC as described below.

Quantification of product and substrate concentrations (formic acid, formaldehyde and glycolic acid) were determined via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature 42° C.). Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 m film thickness, 30 m length). The expected product, glycolic acid, was detected in the media, indicating the performance of corresponding formate activation enzymes. Of the tested forymyl-CoA transferase CaAbfT has the best performance (FIG. 23B).

Since formate activation is a required step for formate utilization, we further evaluated enzymes catalyzing this reaction. Acyl-CoA synthetase (ACS) enzymes are one of the three explored routes for formate activation, which can directly generate formyl-CoA from formate with the consumption of 2 ATP equivalents (ATP is converted to AMP). ACSs evaluated using resting cells showed relatively poor activity as described above, which may be due to limited availability of ATP in the resting cell experiments. Therefore, ACS variants were further evaluated using the new established growing cell platform with ApbHACS overexpression. In this platform, formyl-CoA generated through formate activation enzymes is further condensed with non-toxic substrate acetone to produce 2-hydroxyisobutyrate (2HIB) (FIG. 23C).

The screening of a larger group of formate activation enzymes in actively growing cells was conducted using the M9-LB medium contains 6.78 g/L Na₂HPO₄, 3 g/L KH₂PO₄, 1 g/L NH₄Cl, 0.5 g/L NaCl, 2 mM MgSO₄, 100 μM CaCl₂), 15 μM thiamine-HCl, 10 g/L tryptone, and 5 g/L yeast extract additionally supplemented with the micronutrient solution of Neidhardt⁶⁸. A single colony of the desired strain was cultivated overnight (14-16 hrs) in LB medium with appropriate antibiotics and used as the inoculum (1%) to 50 mL centrifugation tubes containing 5 mL of M9-LB medium. Antibiotics (100 g/mL carbenicillin, 100 g/mL spectinomycin) were included when appropriate. Cultures were then incubated at 30° C. and 250 rpm in a Lab Companion SI-600 rotary shaker (Jeio Tech, Seoul, South Korea) until an OD550 of ˜0.4 was reached, at which point appropriate amounts of inducer(s) (isopropyl β-D-1-thiogalactopyranoside and cumate) and substrates (100 mM acetone and 20 mM formate) were added. Tubes were tightened and incubated for a total of 48 hr post-inoculation. The cells were pelleted by centrifugation and the supernatant analyzed by HPLC as described below. The expected product 2-hydroxyisobutyric acid was detected in the media indicating the performance of corresponding formate activation enzymes. Quantification of product and substrate concentrations (formic acid, acetone and 2HIB) were determined via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature 42° C.). Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 m film thickness, 30 m length).

To test the ACS activity, the two CoA transferases which have good performance on formate activation were also included to serve as positive controls. By screening a larger group of FAEs in actively growing cells, the CoA transferases (CaAbfT and OfFrc) which have a better performance on formate activation than other tested formate activation enzymes. Of the ACSs tested, StACS has the best performance, its activity being comparable to CoA transferases (FIG. 23D).

### Example 16: Formyl-CoA Generation from Formaldehyde

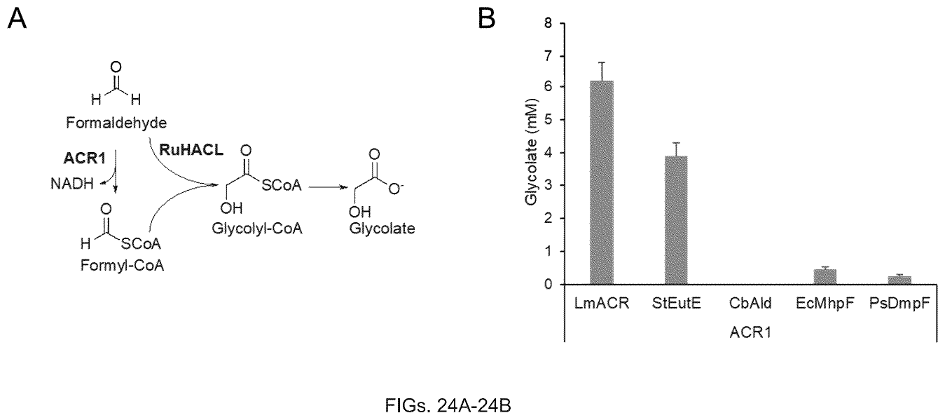

The purpose of this example is to demonstrate the use of various acylating formaldehyde dehydrogenase/acyl-CoA reductase (ACR1) catalyzing interconversion between formaldehyde and formyl-CoA. We used glycolic acid (glycolate) production as proxy, by adding active 2-hydroxyacyl-CoA lyase, HACL from Rhodospirillales bacterium URHD0017 (RuHACL), along with different ACR1 candidates (FIG. 24A).

Cell-free reactions for pathway prototyping contained 50 mM KPi pH 7.4, 4 mM MgCl₂, 0.1 mM TPP, 2.5 mM CoASH, 5 mM NAD⁺, and 50 mM formaldehyde. Individual cell extract loading was around 4.4 g/L protein (⅛ of the reaction volume), and the amount of protein added to each reaction was normalized with BL21(DE3) extract to ˜26 g/L protein (¾ of the reaction volume). Reactions were incubated at room temperature for one hour unless otherwise specified. ¼ of the reaction volume of saturated ammonium sulfate solution acidified with 1% sulfuric acid was added to terminate the reactions. Samples were centrifuged at 20817×g for 15 minutes and the supernatant analyzed by HPLC or GC-MS as described below.

Quantification of product and substrate concentrations (formic acid, formaldehyde and glycolic acid) were determined via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature 42° C.). Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 m film thickness, 30 m length).

We combined extracts of E. coli, prepared as described in the previous example(s), expressing RuHACL and ACR1 variants to rapidly screen for the best performing combinations. Of the ACRs tested, the variant from Listeria monocytogenes (LmACR) was best suited for enabling the pathway. The combination of LmACR and RuHACL enabled the production of 6.2±0.6 mM glycolic acid in 1 hr (FIG. 24B).

### Example 17: Methanol as the Source for Redox (NADH) and Formyl-CoA Generation

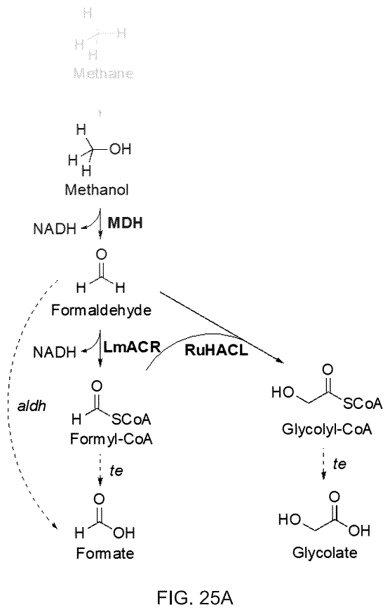

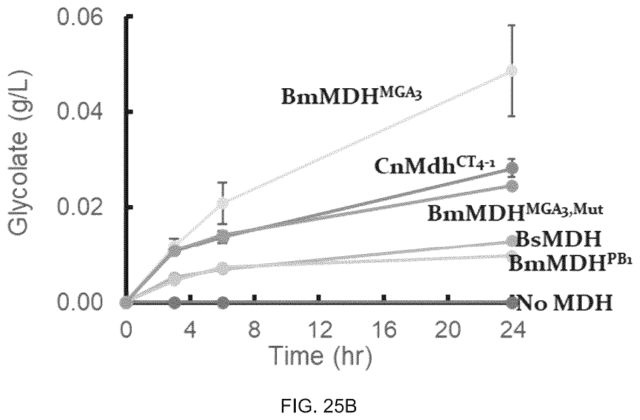

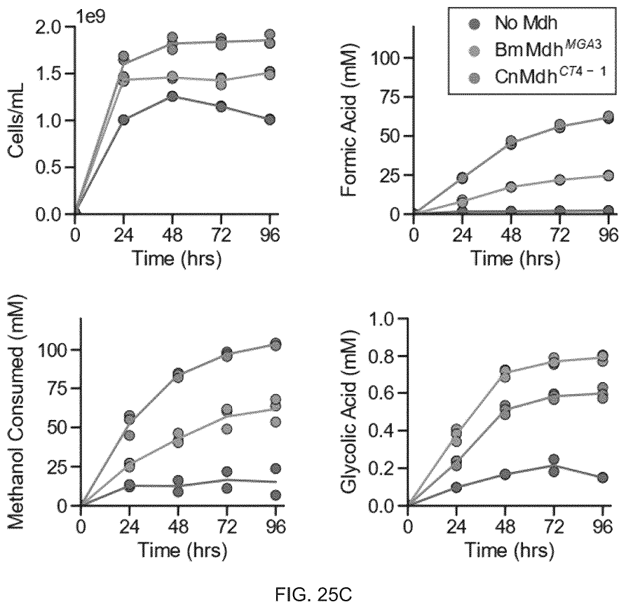

The purpose of this example is to demonstrate the utilization of methanol as source for NADH and formyl-CoA generation demonstrated in vivo with both resting cells and growing cultures. Methanol oxidation to formaldehyde catalyzed by NAD⁺-dependent methanol dehydrogenase generates NADH, which could be used as reducing power for the pathway or energy in the form of ATP when coupled with oxidative phosphorylation. Likewise, formaldehyde oxidation to formyl-CoA also generates one NADH. These two enzymes combined with HACL from Rhodospirillales bacterium URHD0017 (RuHACL) catalyze methanol to glycolyl-CoA, which is readily hydrolyzed to glycolate by endogenously expressed thioesterases (FIG. 25A).

To implement methanol utilization pathway in vivo, we engineered vectors to express RuHACL and the acyl-CoA reductase from Listeria monocytogenes (LmACR) and various methanol dehydrogenase (MDH) candidates. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD), which we expected could compete or interfere with the analysis of our pathway.

Resting-cell prototyping was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 125 mL baffled flasks (Wheaton, Millville, NJ) containing 25 mL of the above media further supplemented with 20 g/L glycerol, 10 g/L tryptone, and 5 g/L yeast extract. A single colony of the desired strain was cultivated overnight (14-16 hrs) in LB medium with appropriate antibiotics and used as the inoculum (1%). Antibiotics (50 g/mL carbenicillin, 50 g/mL spectinomycin) were included when appropriate. Cultures were then incubated at 30° C. and 250 rpm in a Lab Companion SI-600 rotary shaker (Jeio Tech, Seoul, South Korea) until an OD550 of ˜0.4 was reached, at which point appropriate amounts of inducer(s) (isopropyl β-D-1-thiogalactopyranoside and cumate) were added. Flasks were incubated for a total of 24 hrs post-inoculation.

Cells from the above pre-cultures were then centrifuged (5000×g, 22° C.), washed twice with the above minimal media without any carbon source, and resuspended to an optical density ˜10.5 mL of this cell suspension and indicated amounts of carbon source (e.g. formaldehyde) was added to 25 mL Pyrex Erlenmeyer flasks (Corning Inc., Corning, NY) and sealed with foam plugs filling the necks. 25 mM acetone and 5 mM formaldehyde were added at 0 hr, with additional 5 mM formaldehyde added at 1, 2, and 3 hrs Flasks were incubated at 30° C. and 200 rpm in an NBS 124 Benchtop Incubator Shaker (New Brunswick Scientific Co., Inc., Edison, NJ). After incubation at 30° C. for 24 hours, the cells were pelleted by centrifugation and the media analyzed. The expected product glycolic acid was detected in the media, indicating production by the engineered organism.

For the growing-cell experiment, the growth media used was M9 (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) additionally supplemented with 500 mM methanol, 10 g/L tryptone, 5 g/L yeast extract and micronutrient solution of Neidhardt et al. An overnight LB culture of each strain was used to inoculate (1%) a 50 mL closed-cap conical tube (Genesee Scientific Co.) containing 5 mL of the above media further supplemented with appropriate antibiotics (50 μg/mL carbenicillin, 50 μg/mL spectinomycin). After approximately 3 hours, gene expression was induced by addition of 0.04 mM isopropyl β-d-1-thiogalactopyranoside (IPTG) and 0.04 mM cumate. Tubes were incubated at 30° C. and 200 rpm in an NBS 124 Benchtop Incubator Shaker (New Brunswick Scientific Co.). Samples (100 μL) were taken every 24, 48, 72 and 96 hours after inoculation for OD600 measurement and HPLC analysis as described previously.

The resting-cell experiment shows that the combination of various MDHs and LmACR produces formyl-CoA, as indicated by glycolate production as proxy. Of variants tested, BmMDH^{MGA3} gives the best glycolate production of up to 50 mg/L, followed by CnVMDH^{CT4-1} of 30 mg/L under the resting-cell format (FIG. 25B).

The best two enzyme candidates were tested under the growing-cell experiment. The correlation between methanol consumption and cell growth indicates the utilization of methanol as source of energy (ATP) via respiration of excess NADH generation. The result with glycolate production aligns with the result from resting-cell experiments but CnMDH^{CT4-1} shows faster consumption of methanol and accumulation of formate (FIG. 25C). Therefore, BmMDH^{MGA3} is a better choice for the formyl-CoA generation whereas CnMDH^{CT4-1} is a better enzyme for energy generation.

### Example 18: Cell-Free Pathway Prototyping Using Formaldehyde as Starting Substrate

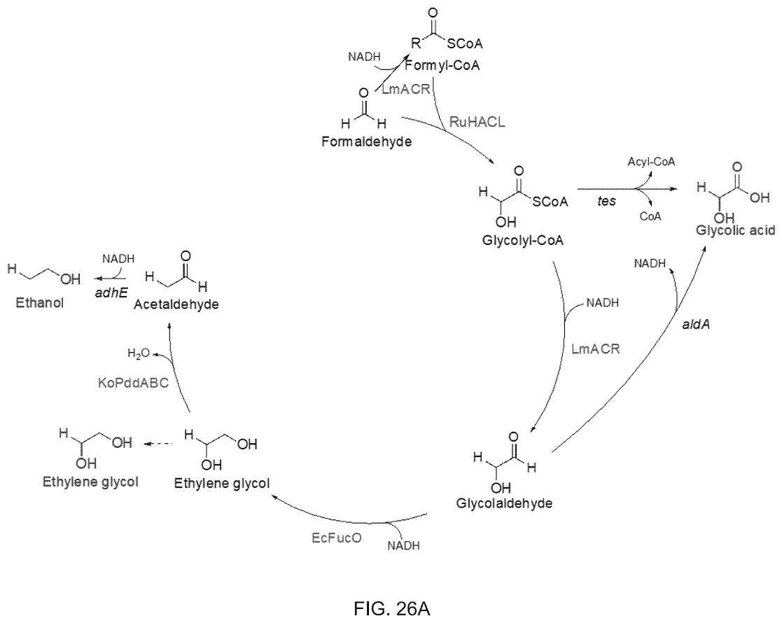

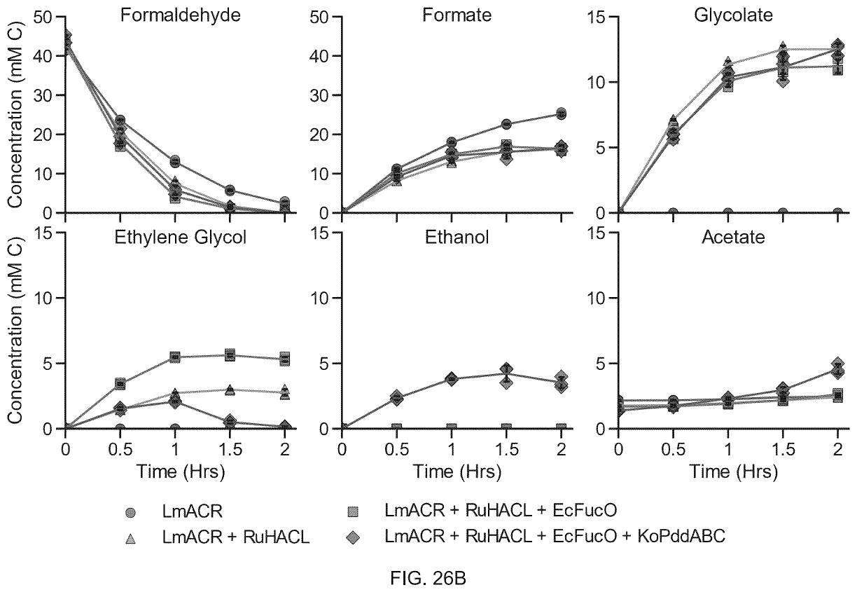

The purpose of this example is to demonstrate the cell-free method for prototyping C1 elongation pathway for formaldehyde as a starting substrate, which could be generated through activation and reduction of carboxylic acid, formate. We prototyped the use of enzymes identified in the previous example as part of a synthetic pathway for the conversion of C1 substrates to multi-carbon products using formaldehyde as the sole carbon source (FIG. 26A). The most immediate two-carbon ligation product of the pathway is glycolic acid, which can be readily hydrolyzed from glycolyl-CoA by endogenously expressed thioesterases. Thus, the synthesis of glycolic acid from formaldehyde using HACL only requires the additional generation of formyl-CoA, which can be accomplished by the use of an acyl-CoA reductase (ACR).

Cell-free reactions for pathway prototyping contained 50 mM KPi pH 7.4, 4 mM MgCl₂, 0.1 mM TPP, 2.5 mM CoASH, 5 mM NAD⁺, and 50 mM formaldehyde. For time course experiments, 0.1 mM coenzyme B12 was added. Individual cell extract loading was around 4.4 g/L protein (⅛ of the reaction volume), and the amount of protein added to each reaction was normalized with BL21(DE3) extract to ˜26 g/L protein (¾ of the reaction volume). Reactions were incubated at room temperature for one hour unless otherwise specified. ¼ of the reaction volume of saturated ammonium sulfate solution acidified with 1% sulfuric acid was added to terminate the reactions. Samples were centrifuged at 20817×g for 15 minutes and the supernatant analyzed by HPLC or GC-MS as described previously.

To demonstrate the utility of the HACL-catalyzed elongation reaction for generating varied chemical functionalities from the resulting 2-hydroxyacyl-CoA product, we included enzymes to extend the LmACR+RuHACL pathway (FIG. 26B). An acyl-CoA reductase is needed to reduce glycolyl-CoA to glycolaldehyde, and upon screening the same set of ACRs for activity on glycolaldehyde, we found that LmACR was also able to act upon glycolaldehyde. To minimize the complexity of the engineered system, we used LmACR in a bifunctional role, catalyzing both the oxidation of formaldehyde to formyl-CoA and the reduction of glycolyl-CoA to glycolaldehyde. As shown in FIG. 26B, LmACR alone resulted in only the conversion of formaldehyde to formate. With the inclusion of RuHACL, glycolate was observed. Glycolaldehyde, however, was not significantly detected as a product, probably due to the presence of endogenous oxidoreductases in the cell extract system, which catalyzed the oxidation of glycolaldehyde to glycolic acid or, to a lesser extent, reduction to ethylene glycol.

The synthesis of the next reduction product, ethylene glycol, was significantly increased by the addition of a cell extract of E. coli overexpressing E. coli FucO, a 1,2-diol oxidoreductase (a 2-fold increase, from 1.37±0.1 mM to 2.73±0.03 mM) (FIG. 26B). Ethylene glycol can be further dehydrated to acetaldehyde by a diol dehydratase. Upon addition of E. coli cell extract expressing diol dehydratase (DDR) from Klebsiella oxytoca, ethanol was detected (1.90±0.03 mM at one hour: FIG. 26B), a product of the reduction of acetaldehyde by endogenous aldehyde reductases, along with a corresponding decrease in ethylene glycol. Synthesis of these varied products (i.e. glycolate, ethylene glycol, ethanol) illustrates the use of the 2-hydroxyacyl-CoA node to readily generate products at varying levels of reduction, chain lengths, and functionalities.

### Example 19: Production of Glycine from Glycolate in Glycine Auxotroph Strain

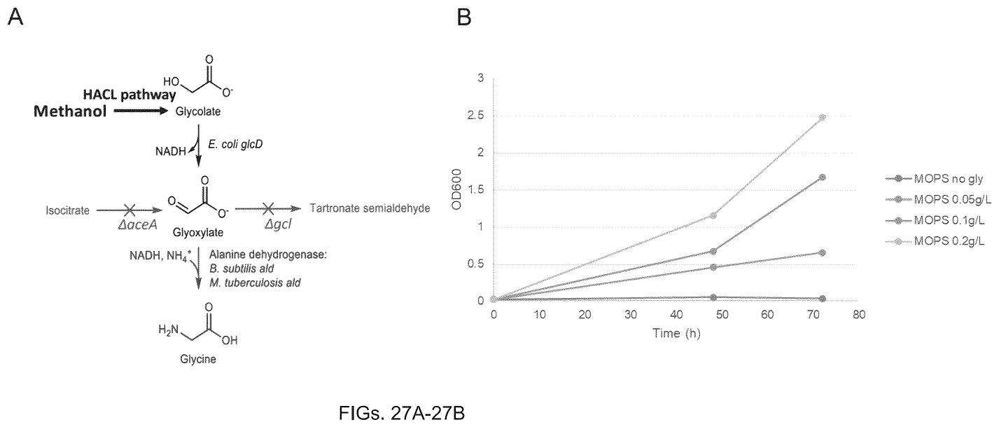

This Example illustrates the alternative route of 2-hydroxyacids to amino acid production when formate is used as the starting carboxylic acid. The demonstration of glycolate conversion to glycine was done through growth-coupled selection of two candidates for alanine dehydrogenases catalyzing glyoxylate reduction to glycine (FIG. 27A). As a host for the selection platform, we engineered a glycine auxotroph strain of E. coli based on MG1655(DE3) with knockouts for glycine production and utilization (ΔaceA Δkbl ΔltaE ΔglyA), which forced the strain to grow only in with the glycine supplementation (FIG. 27A).

For gene deletions, CRISPR is used based on the method developed in Appl. Environ. Microbiol. 81:2506-2514, 2015). First, the host strain is transformed with plasmid pCas, the vector for expression of Cas9 and λ-red recombinase. The resulting strain is grown under 30° C. with L-arabinose for induction of λ-red recombinase expression, and when OD reaches ˜0.6, competent cells are prepared and transformed with pTargetF (AddGene 62226) expressing sgRNA and N20 spacer targeting the locus and template of insertion of target gene. The template is the deleted gene with ˜500 bp sequences homologous with upstream and downstream of the insertion locus, constructed through overlap PCR with usage of Phusion polymerase or synthesized by GenScript (Piscataway, NJ). The way to switch N20 spacer of pTargetF plasmid is inverse PCR with the modified N20 sequence hanging at the 5′ end of primers with usage of Phusion polymerase and followed by self-ligation with usage of T4 DNA ligase and T4 polynucleotide kinase (New England Biolabs, Ipswich, MA, USA). Transformants that grow under 30° C. on solid media (LB+Agar) supplemented with spectinomycin and kanamycin (or other suitable antibiotic) are isolated and screened for the chromosomal gene insert by PCR. The sequence of the gene insert, which is amplified from genomic DNA through PCR using Phusion polymerase, is further confirmed by DNA sequencing. The pTargetF can then be cured through IPTG induction, and pCas can be cured through growth under higher temperature like 37-42° C.

The resulting glycine auxotroph strain was transformed with a vector constitutively expressing alanine dehydrogenase from Mycobacterium tuberculosis (MtAld) or Bacillus subtilis (BsAld). Out of the two candidates, a strain harboring BsAld started to grow with glycolate instead of glycine supplementation (FIG. 27B) indicating glycolate is successfully being converted to glycine via native glcD and heterologously expressed BsAld genes.

### Example 20: Formamide as Substrate for Condensation with Formyl-CoA

This Example demonstrates the implementation of the carboxylic acid (CA) platform using formic acid (formate) as the CA intermediate with C₁ elongation pathways. Amination of formate catalyzed by formamidase gives formamide which can also be used as substituted C₁ aldehyde substrate (Table 8) for the 2-hydroxyacyl-CoA synthase (HACS) reaction. Functional expression and characterization of formamidases from Methylophilus methylotrophus are demonstrated in literature (Eur. J. Biochem. 240:314-322 (1996)).

The subsequent C₁ elongation of formamide catalyzed by HACS forms 2-aminolactoyl-CoA. 2-aminolactoyl-CoA can be utilized as CoA donor for activation of formate using CoA transferases such as AbfT from Clostridium aminobutyricum, or hydrolyzed through the action of a thioesterase such as YciA from E. coli to yield 2-hydroxyglycine. In addition, it can undergo 2-aminolactoyl-phosphate intermediate to generate 2-hydroxyglycine and ATP similar to formic acid activation via phosphate intermediate catalyzed by E. coli Pta and Ack or equivalent enzymes. 2-hydroxyglycine can further be converted to oxamic acid via GlcD from E. coli or similar enzyme, followed by amino acid dehydrogenase reaction to generate diaminoacetic acid. Diaminoacetic acid can be reduced to diaminoethanal catalyzed by various aldehyde dehydrogenases/oxidases. The resulting diaminoethanal can be further aminated via diamine dehydrogenase or transaminase activity to form 1,1,2-ethanetriamine or reduced to form 2,2-diaminoethanol.

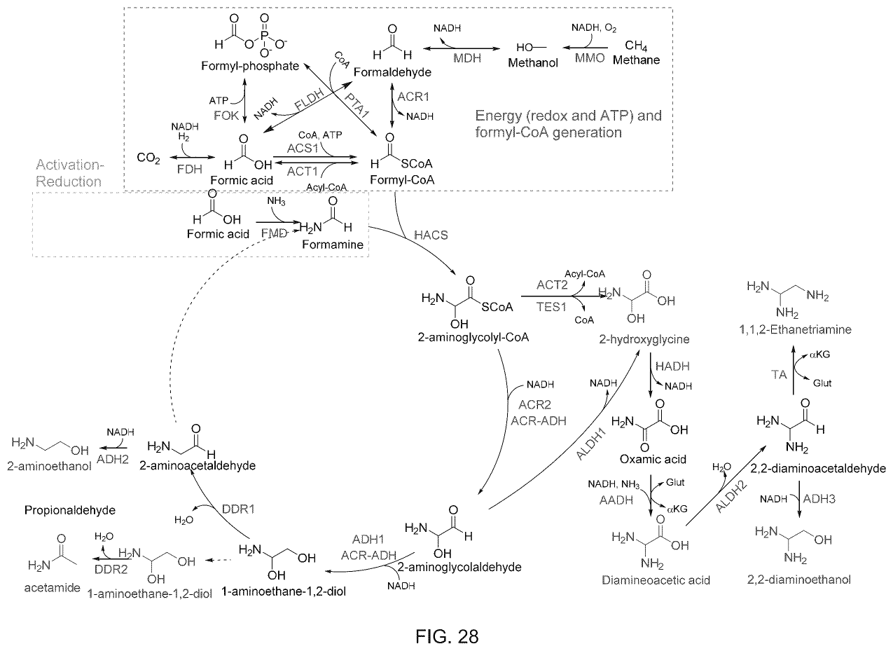

2-aminolactoyl-CoA can be further reduced to 2-aminolactaldehyde via acyl-CoA reductase activity. 2-aminolactaldehyde reduction to glycolamine is catalyzed by E. coli fucO or similar enzymes. Dehydration of glycolamine gives 2-aminoacetaldehyde, catalyzed by diol dehydratases. The resulting 2-aminoacetaldehyde can be fed to the subsequent iteration of C₁ elongation or reduced to ethanolamine catalyzed by alcohol dehydrogenases (FIG. 28 and Table 7).

### Example 21: Construction of Formamidase Expression Vector

This Example demonstrates the design and construction of a vector used for expression of Methylophilus methylotrophus formamidase (FmdA).

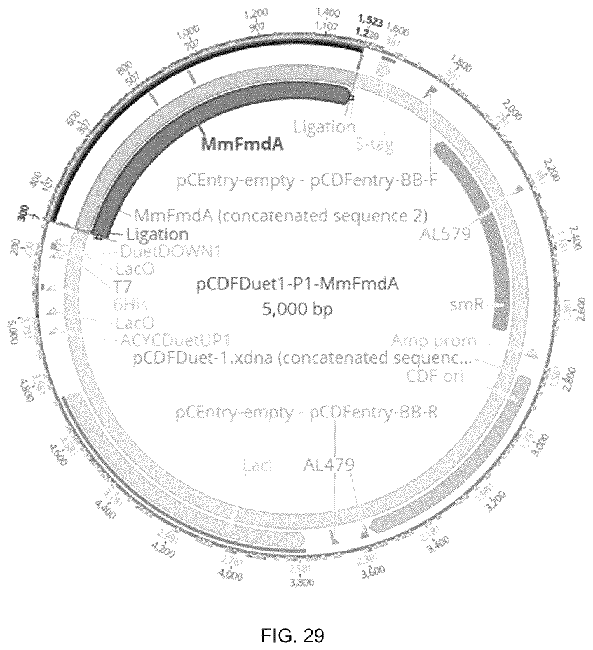

FmdA gene is codon-optimized and synthesized by Twist Biosciences. Then, the gene is amplified through PCR using appropriate primers to append homology on each end for recombination into the vector backbone with e.g., Phusion polymerase (Thermo Scientific, Waltham, MA) to serve as the gene insert. Plasmids are linearized by the appropriate restriction enzymes (New England Biolabs, Ipswich, MA, USA) and recombined with the gene inserts using the In-Fusion HD Eco-Dry Cloning system. The mixture is subsequently transformed into Stellar competent cells. Transformants that grow on solid media (LB+Agar) supplemented with the appropriate antibiotic are isolated and screened for the gene insert by PCR. Plasmids from verified transformants are isolated and the sequence of the gene insert is further confirmed by DNA sequencing. The sequence confirmed plasmids are then introduced to host strain through electroporation (FIG. 29).

### Example 22: Carboxylic Acid Platform Using C₂+ Carboxylic Acids as the CA Intermediate with C1 Elongation Pathways

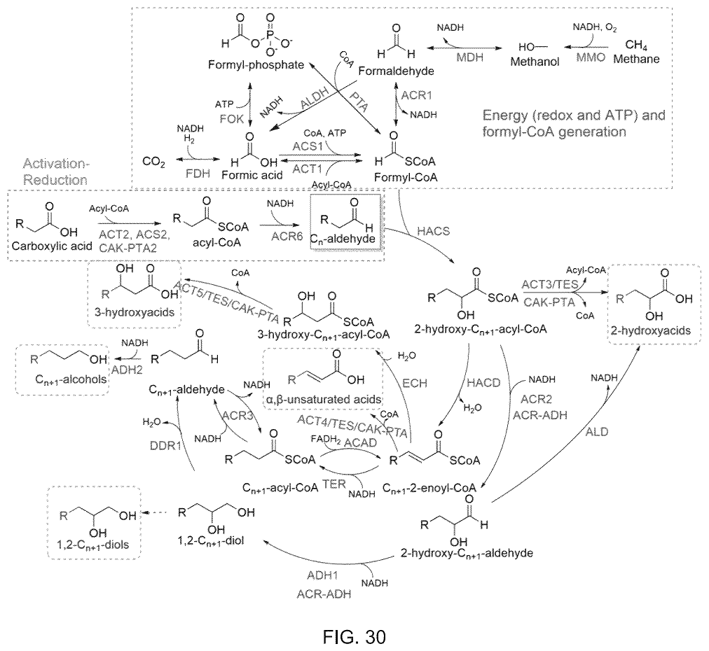

This Example demonstrates the implementation of the carboxylic acid (CA) platform using C₂+ carboxylic acids as the CA intermediate with C₁ elongation pathways. C₂+ carboxylic acids can be supplied as the carbon source. Activation and reduction C₂+ carboxylic acids give C₂+ aldehydes which serve as substrate for the condensation reaction with formyl-CoA (Table 9). The resulting 2-hydroxyacyl-CoAs can be hydrolyzed to the corresponding 2-hydroxyacids (Table 4), which can then be aminated to form 2-amino acids, alkanolamines and diamines. Alternatively, 2-hydroxyacids can go through α-reduction cycle to generate 1,2-diols, methyl ketones, alcohols, 3-hydroxyacids and α,β-unsaturated acids (FIG. 30 and Table 6).

### Example 23: Carboxylic Acid Platform Using Acetic Acid-Derived Acetaldehyde as Substrate for Condensation with Formyl-CoA

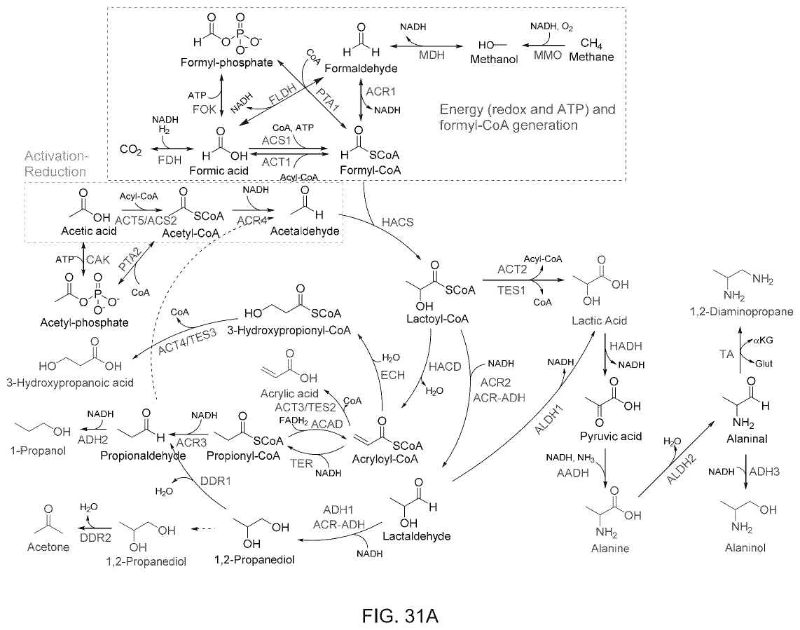

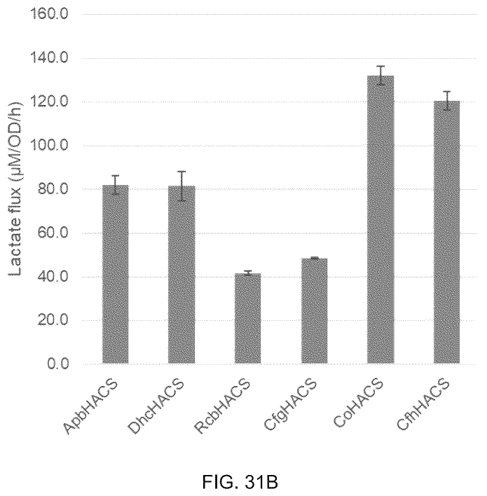

The purpose of this example is to demonstrate the implementation of the carboxylic acid (CA) platform using acetic acid (R═H) as the CA intermediate with C1 elongation pathways (FIG. 31A, Table 5). Acetate can be supplied as the carbon source or generated from electrochemical reduction or microbial conversion from acetogenic bacteria using CO2 as the carbon source. Conversion of CO2 to acetate has been demonstrated using CO2 electrolyzer system with up to 57% carbon selectivity (Hann et al. Nat. Food 3:461-471 (2022)).

Activation of acetic acid to acetyl-CoA and then reduction to acetaldehyde have been well studied in the literatures. Acetic acid can be activated to acetyl-CoA through acetyl-phosphate. First, acetic acid is phosphorylated to acetyl phosphate by acetate kinase from Escherichia coli (Skarstedt et al. J Biol Chem. 251(21):6775-6783 (1976)) and then converted to acetyl-CoA by phosphate acetyltransferase from Escherichia coli (Campos-Bermudez et al. FEBS J. 277(8):1957-1966 (2010)). The other pathway to activate acetic acid is acetyl-CoA synthetase from Escherichia coli (Biochem Biophys Res Commun. 449(3):272-277 (2014)) or succinyl-CoA transferase from Clostridium kluyveri (Söhling et al. Eur J Biochem. 212(1):121-127 (1993)). Finally, acetyl-CoA is reduced to acetaldehyde by acetaldehyde dehydrogenase from Escherichia coli (Song et al. Metab Eng. 35:38-45 (2016)).

C₁ elongation is initiated by formyl-CoA production through energy and formyl-CoA generation steps. 2-hydroxyacyl-CoA synthase (HACS) condenses acetaldehyde and formyl-CoA to produce lactoyl-CoA. Lactoyl-CoA is converted to lactic acid by lactoyl-CoA transferase from Megasphaera elsdenii (Niu et al. ACS Synth Biol. 4(4):378-382 (2015)).

Lactic acid can be further oxidized to pyruvic acid by lactate dehydrogenase from Lacticaseibacillus casei (Gordon et al. Eur J Biochem. 67(2):543-555 (1976)). Pyruvate is reduced to alanine by alanine dehydrogenase from Bacillus subtilis (Yoshida et al. Methods in enzymology 17:176-181 (1970)).

Lactoyl-CoA as the product of condensation can be reduced to 1,2-propanediol (1,2-PDO), acetone and 1-propanol. First, lactoyl-CoA is reduced to lactaldehyde through lactaldehyde dehydrogenase from Salmonella enterica (Niu et al. ACS Synth Biol. 4(4):378-382 (2015)) which can be further reduced to 1,2-PDO by lactaldehyde reductase from E. coli (Niu et al. ACS Synth Biol. 4(4):378-382 (2015)). Dehydration of 1,2-PDO by propanediol dehydratase from Roseburia inulinivorans (LaMattina et al. J Biol Chem. 291(30):15515-15526 (2016)) produces acetone. Moreover, dehydration of 1,2-PDO by diol dehydratase from Salmonella enterica (Bibok et al. J Bacteriol. 179(21):6633-6639(1997)) produces propionaldehyde. The resulting propionaldehyde can be fed to the subsequent iteration of C₁ elongation or reduced to 1-propanol by aldehyde reductase from E. coli (Pick et al. Appl Microbiol Biotechnol. 97(13):5815-5824(2013)).

Lactoyl-CoA can be used to produce unsaturated acids. Lactoyl-CoA is dehydrated to acryloyl-CoA by lactoyl-CoA dehydratase from Anaerotignum propionicum (Kandasamy et al. Appl Microbiol Biotechnol. 97(3):1191-1200(2013)). Acryloyl-CoA is converted to acrylic acid through CoA transferase from Halomonas sp. HTNK1 (Todd et al. Environ Microbiol. 12(2):327-343 (2010)). Moreover, acryloyl-CoA can be hydrolyzed to 3-hydroxypropionyl-CoA and further oxidized to 3-hydroxypropionic acid by acryloyl-CoA hydratase from Halomonas sp. HTNK1 (Todd et al. Environ Microbiol. 12(2):327-343 (2010)). Another pathway for acryloyl-CoA is to be converted to propionyl-CoA by propionyl-CoA synthase from Chloroflexus aurantiacus (Alber et al. J Biol Chem. 277(14):12137-12143 (2002)). Propionyl-CoA is further reduced to propionaldehyde by propanal dehydrogenase from Salmonella enterica (Niu et al. ACS Synth Biol. 4(4):378-382 (2015)).

To implement the C₁ elongation pathway for lactic acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.5 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 20 mM acetaldehyde and 20 mM formate were added at 0 hr and were incubated at 30° C. and 1000 rpm in Digital Microplate Shaker (Fisher Scientific). After incubation at 30° C. for 3 hours, the cells were pelleted by centrifugation and the media was analyzed using HPLC.

The result indicates that CoHACS is the best candidate under the given experimental conditions reaching up to 140 μM/hr per OD600 (FIG. 31B, Table 5).

### Example 24: Carboxylic Acid Platform Using Glycolic Acid-Derived Glycolaldehyde as Substrate for Condensation with Formyl-Coa

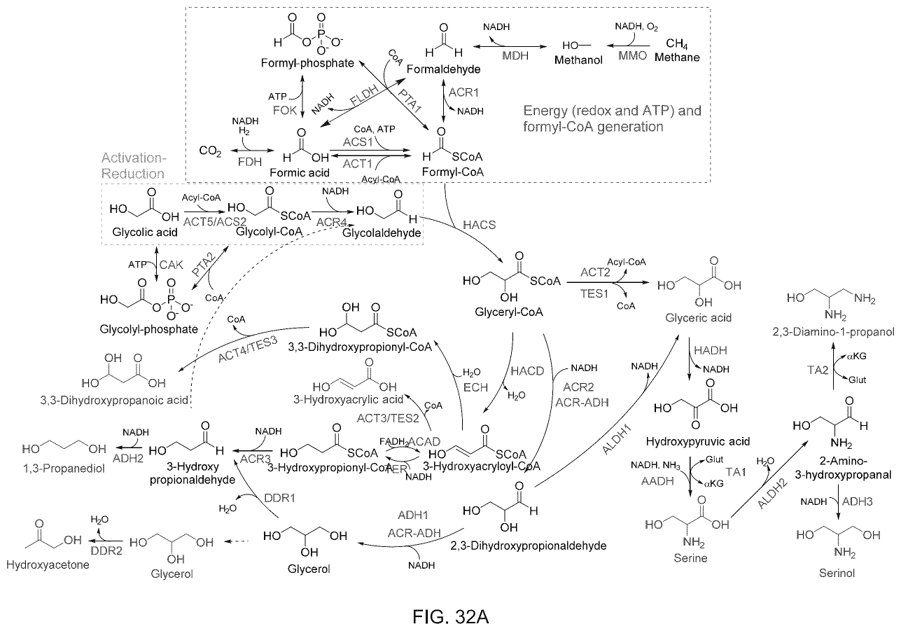

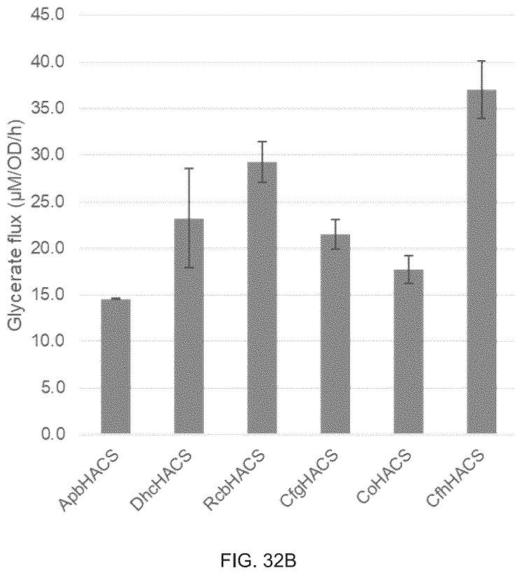

The purpose of this example is to demonstrate the implementation of the carboxylic acid (CA) platform using glycolic acid (R═OH) as the CA intermediate with C1 elongation pathways (FIG. 32A).

Glycolic acid can be activated to glycolyl-CoA through glycolyl-phosphate. First, glycolic acid is phosphorylated to glycolyl-phosphate by kinase and then converted to glycolyl-CoA by phosphotransacylase. The other pathway to activate glycolic acid is CoA synthetase from or CoA transferase. Finally, glycolyl-CoA is reduced to glycolaldehyde by aldehyde dehydrogenase.

C1 elongation is initiated by formyl-CoA production through energy and formyl-CoA generation steps. 2-hydroxyacyl-CoA synthase (HACS) condenses glycolaldehyde and formyl-CoA to produce glyceryl-CoA. Glyceryl-CoA is converted to glyceric acid by acyl-CoA thioesterase.

Glyceric acid can be further oxidized to hydroxypyruvic acid by 2-hydroxyacid dehydrogenase. Hydroxypyruvic acid is reduced to serine by amino dehydrogenase/transaminase. Dehydration of serine produces 2-amino-3-hydroxypropanal which can be further reduced to serinol or aminated to 2,3-diamino-1-propanol.

Glyceryl-CoA as the product of condensation can be reduced to glycerol, hydroxyacetone and 1,3-propanediol. First, glyceryl-CoA is reduced to 2,3-dihydroxypropionaldehyde through aldehyde dehydrogenase which can be further reduced to glycerol by aldehyde reductase. Dehydration of glycerol by diol dehydratase produces hydroxyacetone. Moreover, dehydration of glycerol by diol dehydratase produces 3-hydroxypropionaldehyde. The resulting 3-hydroxypropionaldehyde can be fed to the subsequent iteration of C₁ elongation or reduced to 1,3-propanediol by aldehyde reductase.

Glyceryl-CoA can be used to produce unsaturated acids. Glyceryl-CoA is dehydrated to 3-hydroxyacryloyl-CoA by acyl-CoA dehydratase. 3-Hydroxyacryloyl-CoA is converted to 3-hydroxyacrylic acid through CoA thioesterase. Moreover, 3-hydroxyacryloyl-CoA can be hydrolyzed to 3,3-dihydroxypropionyl-CoA by acyl-CoA dehydratase and further oxidized to 3,3-dihydroxypropionic acid by acyl-CoA thioesterase. Another pathway for 3-hydroxyacryloyl-CoA is to be converted to 3-hydroxypropionyl-CoA by acyl-CoA dehydrogenase. 3-Hydroxypropionyl-CoA is further reduced to 3-hydroxypropionaldehyde by aldehyde dehydrogenase.

To implement the C1 elongation pathway for glyceric acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.5 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 20 mM glycolaldehyde and 20 mM formate were added at 0 hr and were incubated at 30° C. and 1000 rpm in Digital Microplate Shaker (Fisher Scientific). After incubation at 30° C. for 3 hour, the cells were pelleted by centrifugation and the media was analyzed using HPLC.

The result indicates that CfhHACS is the best candidate under the given experimental conditions reaching up to 36 M/hr per OD600 (FIG. 32B, Table 5).

### Example 25: Carboxylic Acid Platform Using Succinic Acid-Derived Succinic Semialdehyde as Substrate for Condensation with Formyl-CoA

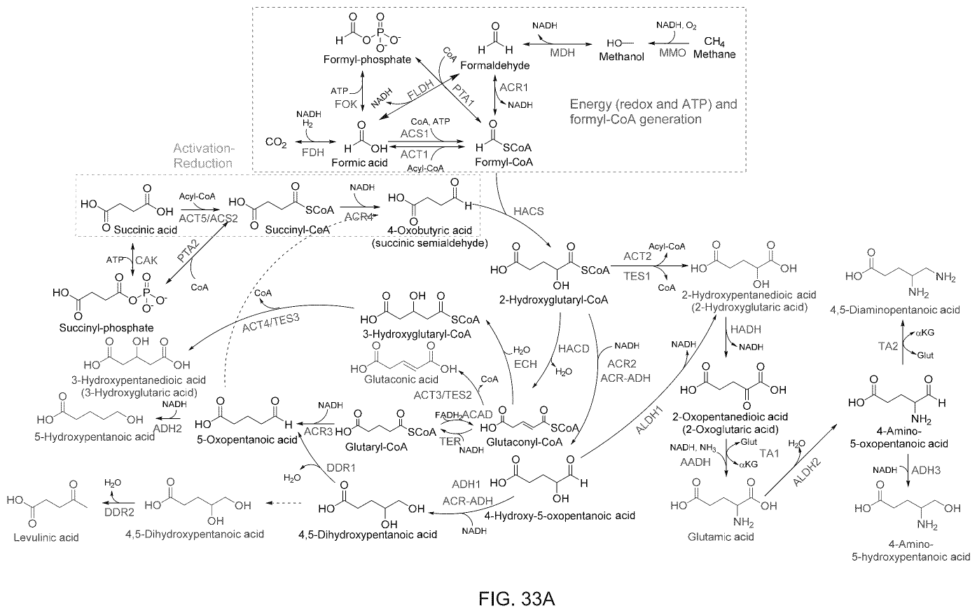

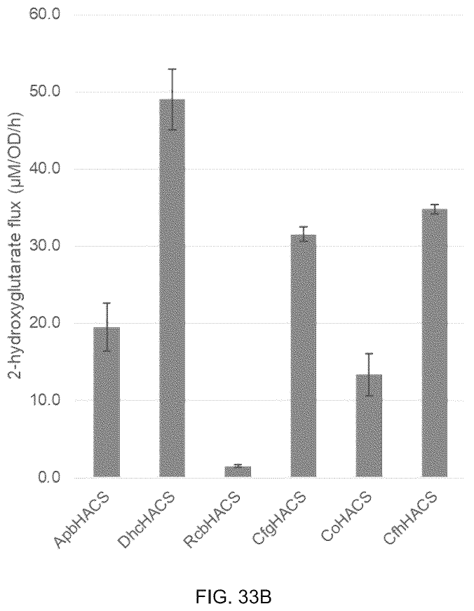

The purpose of this example is to demonstrate the implementation of the carboxylic acid (CA) platform using succinic acid (R═CH₂COOH) as the CA intermediate with C1 elongation pathways (FIG. 33A).

Activation of succinic acid to succinyl-CoA and then reduction to succinic semialdehyde have been well studied in the literature. Succinic acid can be activated to succinyl-CoA through succinyl-phosphate. First, succinic acid is phosphorylated to succinyl-phosphate by kinase and then converted to succinyl-CoA by phosphotransacylase. The other pathway to activate succinic acid is CoA synthetase from E. coli (Yim et al. Nat Chem Biol. 7(7):445-452 (2011)) or CoA transferase. Finally, succinyl-CoA is reduced to succinic semialdehyde by aldehyde dehydrogenase from Clostridium kluyveri (Schwander et al. Science. 354(6314):900-904 (2016)).

C1 elongation is initiated by formyl-CoA production through energy and formyl-CoA generation steps. 2-Hydroxyacyl-CoA synthase (HACS) condenses succinic semialdehyde and formyl-CoA to produce 2-hydroxyglutaryl-CoA. 2-Hydroxyglutaryl-CoA is converted to 2-hydroxyglutaric acid by acyl-CoA thioesterase. 2-Hydroxyglutaric acid can be further oxidized to 2-oxoglutaric acid by 2-hydroxyacid dehydrogenase. 2-Oxoglutaric acid is reduced to glutamic acid by amino dehydrogenase/transaminase. Dehydration of glutamic acid produces 4-amino-5-oxopentanoic acid which can be further reduced to 4-amino-5-hydroxypentanoic acid or aminated to 4,5-diaminopentanoic acid.

2-Hydroxyglutaryl-CoA as the product of condensation can be reduced to 4,5-dihydroxypentanoic acid, levulinic acid and 5-hydroxypentanoic acid. First, 2-hydroxyglutaryl-CoA is reduced to 4-hydroxy-5-oxopentanoic acid through aldehyde dehydrogenase which can be further reduced to 4,5-dihydroxypentanoic acid by aldehyde reductase. Dehydration of 4,5-dihydroxypentanoic acid by diol dehydratase produces levulinic acid. Moreover, dehydration of 4,5-dihydroxypentanoic acid by diol dehydratase produces 5-oxopentanoic acid. The resulting 5-oxopentanoic acid can be fed to the subsequent iteration of C1 elongation or reduced to 5-hydroxypentanoic acid by aldehyde reductase. 2-Hydroxyglutaryl-CoA can be used to produce unsaturated acids. 2-Hydroxyglutaryl-CoA is dehydrated to glutaconyl-CoA by acyl-CoA dehydratase. Glutaconyl-CoA is converted to glutaconic acid through CoA thioesterase. Moreover, glutaconyl-CoA can be hydrolyzed to 3-hydroxyglutaryl-CoA by acyl-CoA dehydratase and further oxidized to 3-hydroxyglutaric acid by acyl-CoA thioesterase. Another pathway for glutaconyl-CoA is to be converted to glutaryl-CoA by acyl-CoA dehydrogenase. Glutaryl-CoA is further reduced to 5-oxopentanoic acid by aldehyde dehydrogenase.

To implement the C1 elongation pathway for 2-hydroxyglutaric acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.5 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 20 mM succinic semialdehyde and 20 mM formate were added at 0 hr and were incubated at 30° C. and 1000 rpm in Digital Microplate Shaker (Fisher Scientific). After incubation at 30° C. for 3 hours, the cells were pelleted by centrifugation and the media was analyzed using HPLC.

The result indicates that DhcHACS is the best candidate under the given experimental conditions reaching up to 50 μM/hr per OD600 (FIG. 33B, Table 5).

### Example 26: Carboxylic Acid Platform Using Isovaleric Acid-Derived Isovaleraldehyde as Substrate for Condensation with Formyl-CoA

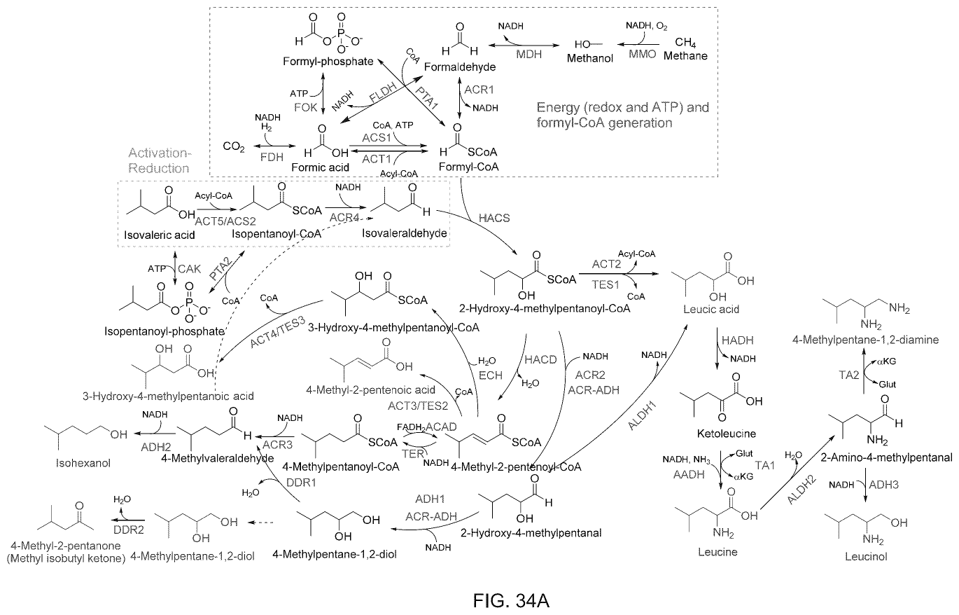

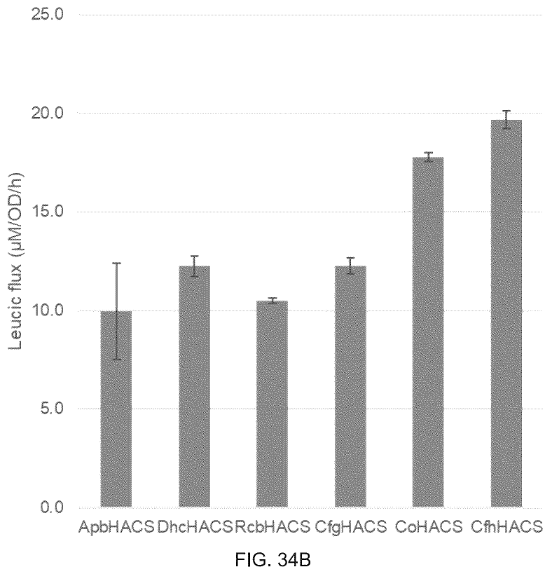

The purpose of this example is to demonstrate the implementation of the carboxylic acid (CA) platform using isovaleric acid (R═CHCH₃CH₃) as the CA intermediate with C1 elongation pathways (FIG. 34A).

Isovaleric acid can be activated to isopentanoyl-CoA through isopentanoyl-phosphate. First, isovaleric acid is phosphorylated to isopentanoyl-phosphate by kinase and then converted to isopentanoyl-CoA by phosphotransacylase. The other pathway to activate isovaleric acid is CoA synthetase or CoA transferase. Finally, isopentanoyl-CoA is reduced to isovaleraldehyde by aldehyde dehydrogenase.

C1 elongation is initiated by formyl-CoA production through energy and formyl-CoA generation steps. 2-Hydroxyacyl-CoA synthase (HACS) condenses isovaleraldehyde and formyl-CoA to produce 2-hydroxy-4-methylpentanoyl-CoA. 2-Hydroxy-4-methylpentanoyl-CoA is converted to leucic acid by acyl-CoA thioesterase.

Leucic acid can be further oxidized to ketoleucine by 2-hydroxyacid dehydrogenase. Ketoleucine is reduced to leucine by amino dehydrogenase/transaminase. Dehydration of leucine produces 2-amino-4-methylpentanal which can be further reduced to leucinol or aminated to 4-methylpentane-1,2-diamine.

2-Hydroxy-4-methylpentanoyl-CoA as the product of condensation can be reduced to 4-methylpentane-1,2-diol, 4-methyl-2-pentanone and isohexanol. First, 2-hydroxy-4-methylpentanoyl-CoA is reduced to 2-hydroxy-4-methylpentanal through aldehyde dehydrogenase which can be further reduced to 4-methylpentane-1,2-diol by aldehyde reductase. Dehydration of 4-methylpentane-1,2-diol by diol dehydratase produces 4-methyl-2-pentanone. Moreover, dehydration of 4-methylpentane-1,2-diol by diol dehydratase produces 4-methylvaleraldehyde. The resulting 4-methylvaleraldehyde can be fed to the subsequent iteration of C1 elongation or reduced to isohexanol by aldehyde reductase.

2-Hydroxy-4-methylpentanoyl-CoA can be used to produce unsaturated acids. 2-Hydroxy-4-methylpentanoyl-CoA is dehydrated to 4-methyl-2-pentenoyl-CoA by acyl-CoA dehydratase. 4-Methyl-2-pentenoyl-CoA is converted to 4-methyl-2-pentenoic acid through CoA thioesterase. Moreover, 4-methyl-2-pentenoyl-CoA can be hydrolyzed to 3-hydroxy-4-methylpentanoyl-CoA by acyl-CoA dehydratase and further oxidized to 3-hydroxy-4-methylpentanoic acid by acyl-CoA thioesterase. Another pathway for 4-methyl-2-pentenoyl-CoA is to be converted to 4-methylpentanoyl-CoA by acyl-CoA dehydrogenase. 4-Methylpentanoyl-CoA is further reduced to 4-methylvaleraldehyde by aldehyde dehydrogenase.

To implement the C1 elongation pathway for leucic acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.5 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 20 mM isovaleraldehyde and 20 mM formate were added at 0 hr and were incubated at 30° C. and 1000 rpm in Digital Microplate Shaker (Fisher Scientific). After incubation at 30° C. for 3 hours, the cells were pelleted by centrifugation and the media was analyzed using HPLC.

The result indicates that CfhHACS is the best candidate under the given experimental conditions reaching up to 20 M/hr per OD600 (FIG. 34B, Table 5).

### Example 27: Carboxylic Acid Platform Using 3-(Methylthio)Propionic Acid-Derived Methional as Substrate for Condensation with Formyl-CoA

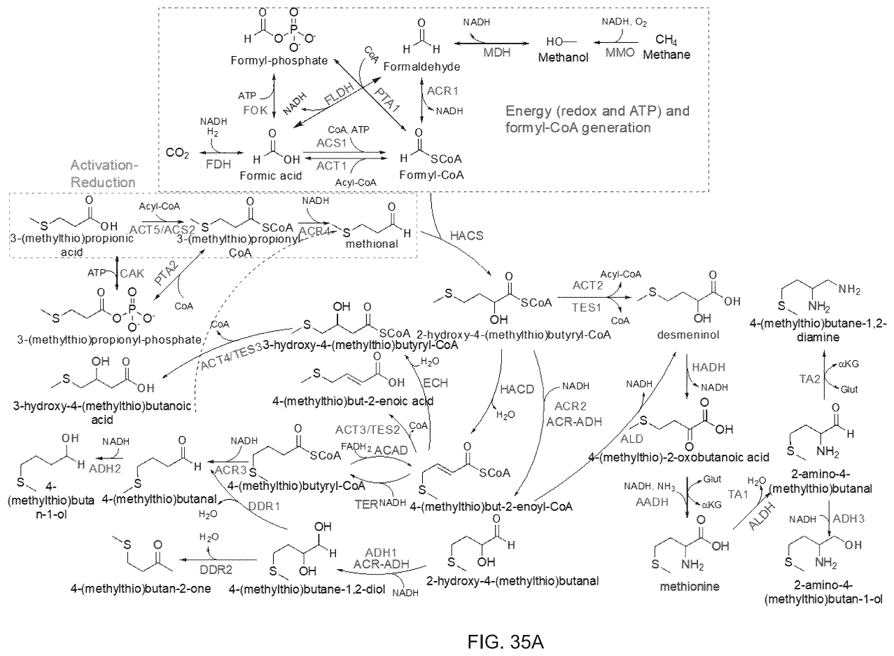

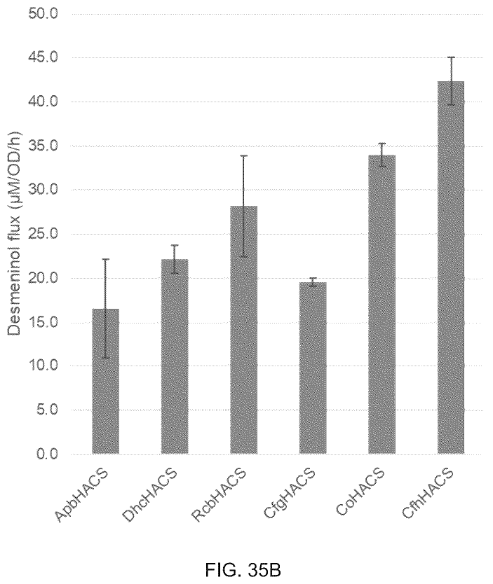

The purpose of this example is to demonstrate the implementation of the carboxylic acid (CA) platform using 3-(methylthio)propionic acid (R═CH3SCH2) as the CA intermediate with C1 elongation pathways (FIG. 35A).

3-(methylthio)propionic acid can be activated to 3-(methylthio)propionyl-CoA through 3-(methylthio)propionyl-phosphate. First, 3-(methylthio)propionic acid is phosphorylated to 3-(methylthio)propionyl-phosphate by kinase and then converted to 3-(methylthio)propionyl-CoA by phosphotransacylase. The other pathway to activate 3-(methylthio)propionic acid is CoA synthetase or CoA transferase. Finally, 3-(methylthio)propionyl-CoA is reduced to methional by aldehyde dehydrogenase.

C1 elongation is initiated by formyl-CoA production through energy and formyl-CoA generation steps. 2-Hydroxyacyl-CoA synthase (HACS) condenses methional and formyl-CoA to produce 2-hydroxy-4-(methylthio)butyryl-CoA. 2-hydroxy-4-(methylthio)butyryl-CoA is converted to desmeninol by acyl-CoA thioesterase.

Desmeninol can be further oxidized to 4-(methylthio)-2-oxobutyric acid by 2-hydroxyacid dehydrogenase. 4-(methylthio)-2-oxobutyric acid is reduced to methionine by amino dehydrogenase/transaminase. Dehydration of methionine produces 2-amino-4-methylthiobutanal which can be further reduced to 2-amino-4-(methylthio)butan-1-ol or aminated to 4-(methylthio)butane-1,2-diamine.

2-hydroxy-4-(methylthio)butyryl-CoA as the product of condensation can be reduced to 4-methylthiobutane-1,2-diol, 4-methylthiobutanal and methylthiobutan-1-ol. First, 2-hydroxy-4-(methylthio)butyryl-CoA is reduced to 2-hydroxy-4-(methylthio)butanal through aldehyde dehydrogenase which can be further reduced to 4-methylthiobutane-1,2-diol by aldehyde reductase. Dehydration of 4-methylthiobutane-1,2-diol by diol dehydratase produces 4-methylthio-2-butanone. Moreover, dehydration of 4-methylthiobutane-1,2-diol by diol dehydratase produces 4-methylthiobutanal. The resulting 4-methylthiobutanal can be fed to the subsequent iteration of C₁ elongation or reduced to methylthiobutan-1-ol by aldehyde reductase.

2-hydroxy-4-(methylthio)butyryl-CoA can be used to produce unsaturated acids. 2-hydroxy-4-(methylthio)butyryl-CoA is dehydrated to 4-(methylthio)but-2-enoyl-CoA by acyl-CoA dehydratase. 4-(methylthio)but-2-enoyl-CoA is converted to 4-(methylthio)but-2-enoic acid through CoA thioesterase. Moreover, 4-(methylthio)but-2-enoyl-CoA can be hydrolyzed to 3-hydroxy-4-methylthiobutyryl-CoA by acyl-CoA dehydratase and further oxidized to 3-hydroxy-4-methylthiobutyric acid by acyl-CoA thioesterase. Another pathway for 4-(methylthio)but-2-enoyl-CoA is to be converted to 4-methylthiobutyryl-CoA by acyl-CoA dehydrogenase. 4-methylthiobutyryl-CoA is further reduced to 4-methylthiobutan-1-ol by aldehyde dehydrogenase.

To implement the C1 elongation pathway for leucic acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.5 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 20 mM methional and 20 mM formate were added at 0 hr and were incubated at 30° C. and 1000 rpm in Digital Microplate Shaker (Fisher Scientific). After incubation at 30° C. for 3 hours, the cells were pelleted by centrifugation and the media was analyzed using HPLC.

The result indicates that CfhHACS is the best candidate under the given experimental conditions reaching up to 43 M/hr per OD600 (FIG. 35B, Table 5).

### Example 28: Carboxylic Acid Platform Using Hydroxypivalic Acid-Derived Hydroxypivaldehyde as Substrate for Condensation with Formyl-CoA

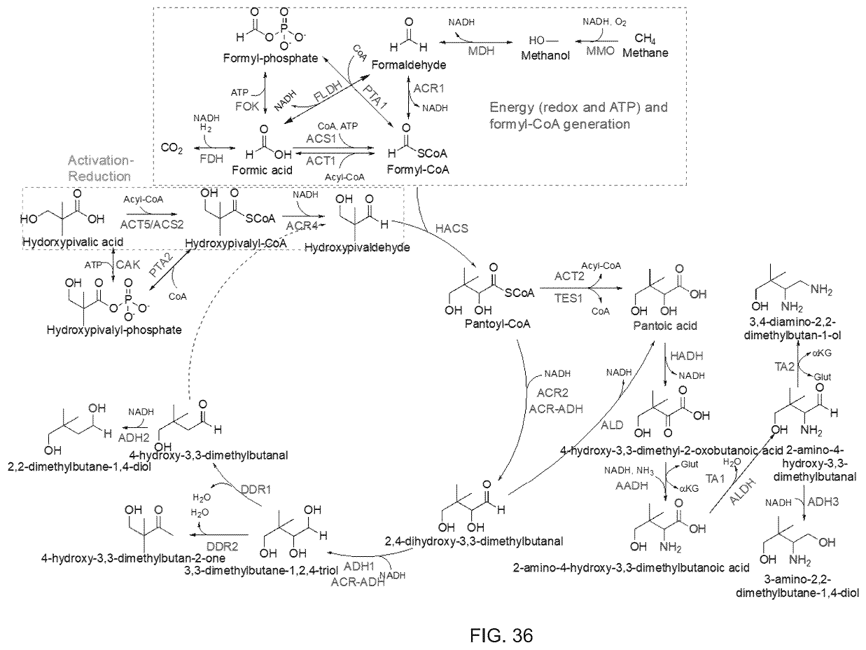

The purpose of this example is to demonstrate the implementation of the carboxylic acid (CA) platform using hydroxypivalic acid (R═(CH₃)₂CH₂OH) as the CA intermediate with C1 elongation pathways (FIG. 36).

Hydroxypivalic acid can be activated to hydroxypivalyl-CoA through hydroxypivalyl-phosphate. First, hydroxypivalic acid is phosphorylated to hydroxypivalyl-phosphate by kinase and then converted to hydroxypivalyl-CoA by phosphotransacylase. The other pathway to activate hydroxypivalic acid is CoA synthetase or CoA transferase. Finally, hydroxypivalyl-CoA is reduced to hydroxypivaldehyde by aldehyde dehydrogenase.

C1 elongation is initiated by formyl-CoA production through energy and formyl-CoA generation steps. 2-Hydroxyacyl-CoA synthase (HACS) condenses methional and formyl-CoA to produce pantoyl-CoA. pantoyl-CoA is converted to pantoic acid by acyl-CoA thioesterase. Pantoic acid can be further oxidized to 4-hydroxy-3,3-dimethyl-2-oxobutanoic acid by 2-hydroxyacid dehydrogenase. 4-hydroxy-3,3-dimethyl-2-oxobutanoic acid is reduced to 2-amino-4-hydroxy-3,3-dimethylbutanoic acid by amino dehydrogenase/transaminase. Dehydration of 2-amino-4-hydroxy-3,3-dimethylbutanoic acid produces 2-amino-4-hydroxy-3,3-dimethylbutanal which can be further reduced to 3-amino-2,2-dimethylbutane-1,4-diol or aminated to 3,4-diamino-2,2-dimethylbutan-1-ol.

Pantoyl-CoA as the product of condensation can be reduced to 2,4-dihydroxy-3,3-dimethylbutanal, 3,3-dimethylbutane-1,2,4-triol and 2,2-dimethylbutane-1,4-diol. First, pantoyl-CoA is reduced to 2,4-dihydroxy-3,3-dimethylbutanal through aldehyde dehydrogenase which can be further reduced to 3,3-dimethylbutane-1,2,4-triol by aldehyde reductase. Dehydration of 3,3-dimethylbutane-1,2,4-triol by diol dehydratase produces 4-hydroxy-3,3-dimethylbutan-2-one. Moreover, dehydration of 3,3-dimethylbutane-1,2,4-triol by diol dehydratase produces 4-hydroxy-3,3-dimethylbutanal. The resulting 4-hydroxy-3,3-dimethylbutanal can be fed to the subsequent iteration of C1 elongation or reduced to 2,2-dimethylbutane-1,4-diol by aldehyde reductase.

### Example 29: Carboxylic Acid Platform Using 3-Hydroxypropionic Acid-Derived 3-Hydroxypropionaldehyde as Substrate for Condensation with Formyl-CoA

The purpose of this example is to demonstrate the implementation of the carboxylic acid (CA) platform using 3-hydroxypropionic acid (R═CH₂OH) as the carboxylic acid (CA) intermediate with C₁ elongation pathways.

3-Hydroxypropionic acid can be activated to 3-hydroxypropionyl-CoA through 3-hydroxypropionyl-phosphate. First, 3-hydroxypropionic acid is phosphorylated to 3-hydroxypropionyl-phosphate by kinase and then converted to 3-hydroxypropionyl-CoA by phosphotransacylase. The other pathway to activate 3-hydroxypropionic acid is CoA synthetase or CoA transferase. Finally, 3-hydroxypropionyl-CoA is reduced to 3-hydroxypropionaldehyde by aldehyde dehydrogenase.

C1 elongation is initiated by formyl-CoA production through energy and formyl-CoA generation steps. 2-Hydroxyacyl-CoA synthase (HACS) condenses 3-hydroxypropionaldehyde and formyl-CoA to produce 2,4-dihydroxybutyryl-CoA. 2,4-Dihydroxybutyryl-CoA is converted to 2,4-dihydroxybutyric acid by acyl-CoA thioesterase.

2,4-Dihydroxybutyric acid can be further oxidized to 2-oxo-4-hydroxybutyric acid by 2-hydroxyacid dehydrogenase. 2-Oxo-4-hydroxybutyric acid is reduced to 2-amino-4-hydroxybutyric acid by amino dehydrogenase/transaminase. Dehydration of 2-amino-4-hydroxybutyric acid produces 2-amino-4-hydroxybutanal which can be further reduced to 2-amino-1,4-butanediol or aminated to 3,4-diamino-1-butanol.

2,4-Dihydroxybutyryl-CoA as the product of condensation can be reduced to 1,2,4-butanetriol, 4-hydroxy-2-butanone and 1,4-butanediol. First, 2,4-dihydroxybutyryl-CoA is reduced to 2,4-dihydroxybutyraldehyde through aldehyde dehydrogenase which can be further reduced to 1,2,4-butanetriol by aldehyde reductase. Dehydration of 1,2,4-butanetriol by diol dehydratase produces 4-hydroxy-2-butanone. Moreover, dehydration of 1,2,4-butanetriol by diol dehydratase produces 4-hydroxybutyraldehyde. The resulting 4-hydroxybutyraldehyde can be fed to the subsequent iteration of C1 elongation or reduced to 1,4-butanediol by aldehyde reductase.

2,4-Dihydroxybutyryl-CoA can be used to produce unsaturated acids. 2,4-Dihydroxybutyryl-CoA is dehydrated to 4-hydroxycrotonyl-CoA by acyl-CoA dehydratase. 4-Hydroxycrotonyl-CoA is converted to 4-hydroxycrotonic acid through CoA thioesterase. Moreover, 4-hydroxycrotonyl-CoA can be hydrolyzed to 3,4-dihydroxybutyryl-CoA by acyl-CoA dehydratase and further oxidized to 3,4-dihydroxybutyric acid by acyl-CoA thioesterase. Another pathway for 4-hydroxycrotonyl-CoA is to be converted to 4-hydroxybutyryl-CoA by acyl-CoA dehydrogenase. 4-Hydroxybutyryl-CoA is further reduced to 4-hydroxybutyraldehyde by aldehyde dehydrogenase.

### Example 30: Carboxylic Acid Platform Using Formic Acid

This example demonstrates the implementation of the carboxylic acid (CA) platform using formic acid (formate) as the CA with C1 elongation pathways. Formate can be supplied as the carbon source, internally generated by the cells through the enzyme pyruvate formate lyase (e.g. PFL1 from Chlamydomonas reinhardtii, pfl from Clostridium pasteurianum, pflB and tdcE from Escherichia coli, pfl from Streptococcus mutans: Nucleic Acids Research 42(1):D459-D471, 2014), generated from electrochemical or enzymatic reduction of CO2. Activation and reduction or amination of formate gives (substituted) C1 aldehydes which serve as substrates for the condensation reaction with formyl-CoA. The resulting 2-hydroxyacyl-CoAs can be hydrolyzed to the corresponding acids, which can then be aminated to form amino acids. Reduction of amino acids to aldehydes gives 2-aminoaldehydes, which could subsequently be aminated to form diamines (catalyzed by diamine dehydrogenase or transaminase) or further reduced to form alkanoamines (2-aminoalcohols) (catalyzed by alcohol dehydrogenase). Alternatively, they can go through α-reduction cycle to generate 1,2-diols and alcohols.

The activation of formate to formyl-CoA is demonstrated using various native and engineered acyl-CoA synthetases (Synth. Biol. 6:1-14, 2021), CoA transferases (J. Biol. Chem. 283: 6519-6529, 2008; ACS Catal. 11:5396-5404, 2021; Nat. Metab. 3:1385-1399, 2021), and carboxylic acid kinase-phosphotransacylase (J. Biol. Chem. 238:2639-2647, 1963). Further reduction of formyl-CoA to formaldehyde is catalyzed by CoA acylating formaldehyde dehydrogenases (e.g. from Listeria monocytogenes: Synth. Biol. 6:1-14, 2021; Nat. Metab. 3:1385-1399, 2021).

When formaldehyde is generated from formyl-CoA, the HACL from Rhodospirillales bacterium URHD0017 can be used to condense formaldehyde with formyl-CoA forming glycolyl-CoA. Glycolyl-CoA can be utilized as CoA donor for activation of formate as demonstrated using AbfT from Clostridium aminobutyricum, or hydrolyzed through the action of a thioesterase such as YciA from E. coli (ACS Catal. 11:5396-5404, 2021) to yield glycolic acid (glycolate). In addition, it can undergo glycolyl-phosphate intermediate to generate glycolate and ATP similar to formic acid activation via phosphate intermediate catalyzed by E. coli Pta and Ack or equivalent enzymes. Glycolate can further be converted to glyoxylate via GlcD from E. coli, followed by alanine dehydrogenase reaction to generate glycine, one of the essential amino acids for living organisms. Multiple alanine dehydrogenases have activity with glyoxylate to yield glycine (J. Bacteriol. 194:1045-1054, 2012; Biochemistry 20:5650-5655, 1981). Glycine can be reduced to aminoacetaldehyde catalyzed by various aldehyde dehydrogenases/oxidases. The resulting aminacetaldehyde can be further aminated via diamine dehydrogenase or transaminase activity to form ethylene diamines or reduced to form ethanolamine.

Glycolyl-CoA can be further reduced to glycolaldehyde via acyl-CoA reductase. In this case the same enzyme (LmACR) used for reduction of formyl-CoA to formaldehyde can catalyze reduction of glycolyl-CoA to glycolaldehyde (Nat. Metab. 3:1385-1399 (2021)). Glycolaldehyde reduction to ethylene glycol is catalyzed by E. coli fucO. Dehydration of ethylene glycol gives acetaldehyde, catalyzed by PddABC from Klebsiella oxytoca. The resulting acetaldehyde can be fed to the subsequent iteration of C₁ elongation or reduced to ethanol catalyzed by E. coli adhE.

### Example 31: Conversion of Formate to Ethylene Glycol

This Example demonstrates the implementation of the carboxylic acid (CA) platform using formic acid (formate) as the CA intermediate with C1 elongation pathways. The activation of formate to formyl-CoA is demonstrated using various native and engineered acyl-CoA synthetases (Synth. Biol. 6:1-14, 2021) and CoA transferases (J. Biol. Chem. 283: 6519-6529, 2008; ACS Catal. 11:5396-5404, 2021; Nat. Metab. 3:1385-1399, 2021). Further reduction of formyl-CoA to formaldehyde is catalyzed by CoA acylating formaldehyde dehydrogenases (e.g., from Listeria monocytogenes: Synth. Biol. 6:1-14, 2021; Nat. Metab. 3:1385-1399, 2021).

When formaldehyde is generated from formyl-CoA, the HACL from Rhodospirillales bacterium URHD0017 (RuHACL) or beach sand metagenome ApbHACS (UniProt accession: A0A3C0TX30) is used to condense formaldehyde with formyl-CoA forming glycolyl-CoA. Glycolyl-CoA can be further reduced to glycolaldehyde via acyl-CoA reductase. In this case the same enzyme (LmACR) used for reduction of formyl-CoA to formaldehyde can catalyze reduction of glycolyl-CoA to glycolaldehyde (Nat. Metab. 3:1385-1399, 2021). Glycolaldehyde reduction to ethylene glycol is catalyzed by E. coli fucO.

Expression of selected enzyme variants was achieved using plasmid-based gene expression by cloning the desired gene(s) into pETDuet-1 or pCDFDuet-1 (Novagen, Darmstadt, Germany) digested with appropriate restriction enzymes and by utilizing In-Fusion cloning technology (Clontech Laboratories, Inc., Mountain View, CA). Linear DNA fragments for insertion were created via PCR of the open reading frame of interest (for genes native to E. coli) or by gene synthesis of the codon optimized gene. Genes were synthesized by GeneArt (Life Technologies, Carlsbad, CA). Resulting in-Fusion reaction products were used to transform E. coli Stellar cells (Clontech Laboratories, Inc., Mountain View, CA), and clones identified by PCR screening were further confirmed by DNA sequencing.

Overnight cultures of the expression strains were grown in LB, which was used to inoculate 25 mL TB medium in a 250 mL baffled flask at 1%. The culture was grown at 30° C. and 250 rpm in an orbital shaker until OD550 reached 0.4-0.6, at which point expression was induced with 0.1 mM IPTG. 24 hours post inoculation, cells were harvested by centrifugation. The cell pellets were washed once with cold 9 g/L NaCl solution and stored at −80° C. until needed. Antibiotics were included where appropriate at the following concentrations: ampicillin (100 μg/mL), carbenicillin (50 μg/mL), and spectinomycin (50 μg/mL).

For protein purification, E. coli cell pellets expressing the desired his-tagged enzymes were prepared as described above. The frozen cell pellets were resuspended in cold lysis buffer (50 mM NaPi pH 7.4, 300 mM NaCl, 10 mM imidazole, 0.1% Triton-X 100) to an approximate OD550 of 40, to which 1 mg/mL of lysozyme and 250 U of Benzonase nuclease was added. The mixture was further treated by sonication on ice using a Branson Sonifier 250 (5 minutes with a 25% duty cycle and output control set at 3), and centrifuged at 7500×g for 15 minutes at 4° C. The supernatant was applied to a chromatography column containing 1 mL TALON metal affinity resin (Clontech Laboratories, Inc., Mountain View, CA), which had been pre-equilibrated with the lysis buffer. The column was then washed first with 10 mL of the lysis buffer and then twice with 20 mL of wash buffer (50 mM NaPi pH 7.4, 300 mM NaCl, 20 mM imidazole). The his-tagged protein of interest was eluted with 1-2 applications of 4 mL elution buffer (50 mM NaPi pH 7.4, 300 mM NaCl, 250 mM imidazole). The eluate was collected and applied to a 10,000 MWCO Amicon ultrafiltration centrifugal device (Millipore, Billerica, MA), and the concentrate (˜100 μL) was washed twice with 4 mL of 50 mM KPi pH 7.4 for desalting. Protein concentrations were estimated by the Bradford method. Purified protein was saved in 20 μL aliquots at −80° C. until needed.

SDS-PAGE was performed using NuPAGE 12% Bis-Tris Protein Gels with SDS running buffer and stained with SimplyBlue SafeStain according to manufacturer protocols (ThermoFisher Scientific, Waltham, MA).

In vitro purified enzyme reactions for products from formate as a sole carbon source was comprised of 200 mM KPi pH 8.0, 5 mM MgCl₂, 0.2 mM TPP, 6 mM NADH, 2 mM succinyl-CoA, 2 μM ApbHACS or RuHACL^{G390N}, 2 μM OfFrc, 4 μM LmACR, 2 μM FucO and 50 mM sodium formate. Reactions were incubated at 30° C. for 24 hours unless otherwise specified. 1/20 of the reaction volume of 10 M NaOH solution was added to terminate the reactions. After 30 min hydrolysis, 1/20 of the reaction volume of 10 N H₂SO₄ was added to neutralize the pH. Samples were centrifuged at 20817×g for 15 minutes and the supernatant analyzed by HPLC or GC-MS as described below.

Quantification of product and substrate concentrations (formic acid, formaldehyde, glycolic acid and ethylene glycol) was performed via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature 42° C.). Compound identification and analysis was performed by GC-MS using an Agilent 7890B Series Custom Gas Chromatography system equipped with a 5977B Inert Plus Mass Selective Detector (for identification) and an Agilent HP-INNOWax Columns (0.25 mm internal diameter, 0.25 m film thickness, 30 m length).

By using the best identified ACR enzyme LmACR*, ethylene glycol titer was improved 85% compared to the wide type LmACR. By adding more LmACR*, providing more reducing equivalent NADH and more CoA donor, the ethylene glycol titer was further increased to 145.8 mg/L. The in vitro experiments demonstrate that LmACR and NADH/reducing equivalents have a significant impact on formate conversion to ethylene glycol.

### Example 32: Carboxylic Acid Platform Using Propionic Acid-Derived Propionaldehyde as Substrate for Condensation with Formyl-CoA

The purpose of this example is to demonstrate the implementation of the carboxylic acid (CA) platform using propionic acid (R═CH₃) as the CA intermediate with C1 elongation pathways.

Activation of propionic acid to propionyl-CoA and then reduction to propionaldehyde have been well studied in the literatures. Propionic acid can be activated to propionyl-CoA through propionyl-phosphate. First, propionic acid is phosphorylated to propionyl-phosphate by propionate kinase from Escherichia coli (Hesslinger et al. Mol Microbiol. 27(2):477-492(1998)) and then converted to propionyl-CoA by phosphate acetyltransferase from Escherichia coli (Hesslinger et al. Mol Microbiol. 27(2):477-492(1998)). The other pathway to activate propionic acid is propionyl-CoA synthetase from Escherichia coli (Guo et al. Prikl Biokhim Mikrobiol. 48(3):289-293(2012)) or succinyl-CoA transferase from Escherichia coli (Haller et al. Biochemistry. 39(16):4622-4629(2000)). Finally, propionyl-CoA is reduced to propionaldehyde by propanal dehydrogenase from Salmonella enterica (Niu et al. ACS Synth Biol. 4(4):378-382(2015)).

C1 elongation is initiated by formyl-CoA production through energy and formyl-CoA generation steps. 2-hydroxyacyl-CoA synthase (HACS) condenses propionaldehyde and formyl-CoA to produce 2-hydroxybutyryl-CoA. 2-Hydroxybutyryl-CoA is converted to 2-hydroxybutyric acid by acyl-CoA thioesterase from Escherichia coli (Lee et al. Biochem Biophys Res Commun. 231(2):452-456(1997)).

2-Hydroxybutyric acid can be further oxidized to 2-oxobutyric acid by lactate dehydrogenase from Desulfovibrio vulgaris (Ogata et al. J Biochem. 89(5):1423-1431 (1981)). 2-Oxobutyric acid is reduced to 2-aminobutyric acid by phenylalanine dehydrogenase from Thermoactinomyces intermedius (Kataoka et al. J Biochem. 116(4):931-936 (1994)). 2-Hydroxybutyryl-CoA as the product of condensation can be reduced to 1,2-butanediol (1,2-BDO), 2-butanone and 1-butanol. First, 2-hydroxybutyryl-CoA is reduced to 2-hydroxybutyraldehyde through aldehyde dehydrogenase from Salmonella enterica (Niu et al. ACS Synth Biol. 4(4):378-382 (2015)) which can be further reduced to 1,2-BDO by aldehyde reductase from E. coli (Niu et al. ACS Synth Biol. 4(4):378-382 (2015)). Dehydration of 1,2-BDO by diol dehydratase produces 2-butanone. Moreover, dehydration of 1,2-BDO by diol dehydratase from Klebsiella oxytoca (Toraya et al. J Biochem. 144(4):437-446 (2008)) produces butyraldehyde. The resulting butyraldehyde can be fed to the subsequent iteration of C₁ elongation or reduced to 1-butanol by aldehyde reductase from E. coli (Pick et al. Appl Microbiol Biotechnol. 97(13):5815-5824(2013)).

2-Hydroxybutyryl-CoA can be used to produce unsaturated acids. 2-Hydroxybutyryl-CoA is dehydrated to crotonyl-CoA by acyl-CoA dehydratase from Anaerotignum propionicum (Kandasamy et al. Appl Microbiol Biotechnol. 97(3):1191-1200(2013)). Crotonyl-CoA is converted to crotonic acid through crotonyl-CoA thioesterase from Emergencia timonensis (Buffa et al. Nat Microbiol. 7(1):73-86 (2022)). Moreover, crotonyl-CoA can be hydrolyzed to 3-hydroxybutyryl-CoA by acyl-CoA dehydratase from Clostridium acetobutylicum (Waterson et al. Methods Enzymol. 71 Pt C:421-430 (1981)) and further oxidized to 3-hydroxybutyric acid by acyl-CoA thioesterase from E. coli (Tseng et al. Appl Environ Microbiol. 75(10):3137-3145 (2009)). Another pathway for crotonyl-CoA is to be converted to butyryl-CoA by butyryl-CoA dehydrogenase from Clostridium kluyveri (Li et al. J Bacteriol. 190(3):843-850 (2008)). Butyryl-CoA is further reduced to butyraldehyde by aldehyde dehydrogenase from Clostridium beijerinckii (Yan et al. Appl Environ Microbiol. 56(9):2591-2599 (1990)).

To implement the C₁ elongation pathway for 2-hydroxybutyric acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 g/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 5 mM propionaldehyde and 20 mM formate were added at 0 hr and were incubated at 30° C. and 1000 rpm in Digital Microplate Shaker (Fisher Scientific). After incubation at 30° C. for 1 hour, the cells were pelleted by centrifugation and the media was analyzed using HPLC.

The result indicates that AbpHACS is the best candidates under the given experimental conditions reaching up to 20 μM/hr per OD600.

### Example 33: Carboxylic Acid Platform Using Butyric Acid-Derived Butyraldehyde as Substrate for Condensation with Formyl-CoA

The purpose of this example is to demonstrate the implementation of the carboxylic acid (CA) platform using butyric acid (R═CH₂CH₃) as the CA intermediate with C1 elongation pathways.

Activation of butyric acid to butyryl-CoA and then reduction to butyraldehyde have been well studied in the literatures. Butyric acid can be activated to butyryl-CoA through butyryl-phosphate. First, butyric acid is phosphorylated to butyryl-phosphate by butyrate kinase from Clostridium acetobutylicum (Cary et al. J Bacteriol. 170(10):4613-4618 (1988)) and then converted to butyryl-CoA by phosphotransbutyrylase from Clostridium acetobutylicum (Wiesenborn et al. Appl Environ Microbiol. 55(2):317-322 (1989)). The other pathway to activate butyric acid is CoA synthetase from Pseudomonas putida (Rand et al. Nat Microbiol. 2(12):1624-1634 (2017)) or CoA transferase from Clostridium kuyveri (Seedorf et al. Proc Natl Acad Sci USA. 105(6):2128-2133 (2008)). Finally, butyryl-CoA is reduced to butyraldehyde by aldehyde dehydrogenase from Clostridium acetobutylicum (Fontaine et al. J Bacteriol. 184(3):821-830 (2002)).

C1 elongation is initiated by formyl-CoA production through energy and formyl-CoA generation steps. 2-hydroxyacyl-CoA synthase (HACS) condenses butyraldehyde and formyl-CoA to produce 2-hydroxypentanoyl-CoA. 2-Hydroxypentanoyl-CoA is converted to 2-hydroxyvaleric acid by acyl-CoA thioesterase.

2-Hydroxyvaleric acid can be further oxidized to 2-oxovaleric acid by 2-hydroxyacid dehydrogenase. 2-Oxovaleric acid is reduced to 2-aminovaleric acid by amino dehydrogenase/transaminase.

2-Hydroxypentanoyl-CoA as the product of condensation can be reduced to 1,2-pentanediol, 2-pentanone and valeraldehyde. First, 2-hydroxypentanoyl-CoA is reduced to 2-hydroxypentanal through aldehyde dehydrogenase which can be further reduced to 1,2-pentanediol by aldehyde reductase. Dehydration of 1,2-pentanediol by diol dehydratase produces 2-pentanone. Moreover, dehydration of 1,2-pentanediol by diol dehydratase produces valeraldehyde. The resulting valeraldehyde can be fed to the subsequent iteration of C₁ elongation or reduced to 1-pentanol by aldehyde reductase.

2-Hydroxypentanoyl-CoA can be used to produce unsaturated acids. 2-Hydroxypentanoyl-CoA is dehydrated to 2-pentenoyl-CoA by acyl-CoA dehydratase. 2-Pentenoyl-CoA is converted to 2-pentenoic acid through CoA thioesterase. Moreover, 2-pentenoyl-CoA can be hydrolyzed to 3-hydroxypentanoyl-CoA by acyl-CoA dehydratase from Pseudomonas putida (Rand et al. Nat Microbiol. 2(12):1624-1634 (2017)) and further oxidized to 3-hydroxyvaleric acid by acyl-CoA thioesterase. Another pathway for 2-pentenoyl-CoA is to be converted to pentanoyl-CoA by acyl-CoA dehydrogenase from E. coli (Campbell et al. J Bacteriol. 184(13):3759-3764 (2002)). Pentanoyl-CoA is further reduced to valeraldehyde by aldehyde dehydrogenase.

### Example 34: Carboxylic Acid Platform Using Lactic Acid-Derived Lactaldehyde as Substrate for Condensation with Formyl-CoA

The purpose of this example is to demonstrate the implementation of the carboxylic acid (CA) platform using lactic acid (R═CH30H) as the CA intermediate with C₁ elongation pathways.

Lactic acid can be activated to lactoyl-CoA through lactoyl-phosphate. First, lactic acid is phosphorylated to lactoyl-phosphate by kinase and then converted to lactoyl-CoA by phosphotransacylase. The other pathway to activate lactic acid is CoA synthetase from or CoA transferase from Megasphaera elsdenii (Niu et al. ACS Synth Biol. 4(4):378-382 (2015)). Finally, lactoyl-CoA is reduced to lactaldehyde by aldehyde dehydrogenase from Salmonella enterica (Niu et al. ACS Synth Biol. 4(4):378-382 (2015)).

C1 elongation is initiated by formyl-CoA production through energy and formyl-CoA generation steps. 2-Hydroxyacyl-CoA synthase (HACS) condenses lactaldehyde and formyl-CoA to produce 2,3-dihydroxybutyryl-CoA. 2,3-Dihydroxybutyryl-CoA is converted to 2,3-dihydroxybutyric acid by acyl-CoA thioesterase.

2,3-Dihydroxybutyric acid can be further oxidized to 2-oxo-3-hydroxybutyric acid by 2-hydroxyacid dehydrogenase. 2-Oxo-3-hydroxybutyric acid is reduced to 2-amino-3-hydroxybutyric acid by amino dehydrogenase/transaminase. Dehydration of 2-amino-3-hydroxybutyric acid produces 2-amino-3-hydroxybutanal which can be further reduced to 2-amino-1,3-butanediol or aminated to 3,4-diamino-2-butanol.

2,3-Dihydroxybutyryl-CoA as the product of condensation can be reduced to 1,2,3-butanetriol, acetion and 1,3-butanediol. First, 2,3-dihydroxybutyryl-CoA is reduced to 2,3-dihydroxybutyraldehyde through aldehyde dehydrogenase which can be further reduced to 1,2,3-butanetriol by aldehyde reductase. Dehydration of 1,2,3-butanetriol by diol dehydratase produces acetion. Moreover, dehydration of 1,2,3-butanetriol by diol dehydratase produces 3-hydroxybutyraldehyde. The resulting 3-hydroxybutyraldehyde can be fed to the subsequent iteration of C₁ elongation or reduced to 1,3-butanediol by aldehyde reductase.

2,3-Dihydroxybutyryl-CoA can be used to produce unsaturated acids. 2,3-Dihydroxybutyryl-CoA is dehydrated to 3-hydroxycrotonyl-CoA by acyl-CoA dehydratase. 3-Hydroxycrotonyl-CoA is converted to 3-hydroxycrotonic acid through CoA thioesterase. Moreover, 3-hydroxycrotonyl-CoA can be hydrolyzed to 3,3-dihydroxybutyryl-CoA by acyl-CoA dehydratase and further oxidized to 3,3-dihydroxybutyric acid by acyl-CoA thioesterase. Another pathway for 3-hydroxycrotonyl-CoA is to be converted to 3-hydroxybutyryl-CoA by acyl-CoA dehydrogenase. 3-Hydroxybutyryl-CoA is further reduced to 3-hydroxybutyraldehyde by aldehyde dehydrogenase.

### Example 35: Carboxylic Acid Platform Using Glyceric Acid-Derived Glyceraldehyde as Substrate for Condensation with Formyl-CoA

The purpose of this example is to demonstrate the implementation of the carboxylic acid (CA) platform using glyceric acid (R═OHCH₂OH) as the CA intermediate with C₁ elongation pathways.

Glyceric acid can be activated to glyceryl-CoA through glyceryl-phosphate. First, glyceric acid is phosphorylated to glyceryl-phosphate by kinase and then converted to glyceryl-CoA by phosphotransacylase. The other pathway to activate glyceric acid is CoA synthetase or CoA transferase. Finally, glyceryl-CoA is reduced to glyceraldehyde by aldehyde dehydrogenase.

C1 elongation is initiated by formyl-CoA production through energy and formyl-CoA generation steps. 2-Hydroxyacyl-CoA synthase (HACS) condenses glyceraldehyde and formyl-CoA to produce 2,3,4-trihydroxybutyryl-CoA. 2,3,4-Trihydroxybutyryl-CoA is converted to 2,3,4-trihydroxybutyric acid by acyl-CoA thioesterase.

2,3,4-Trihydroxybutyric acid can be further oxidized to 2-oxo-3,4-dihydroxybutyric acid by 2-hydroxyacid dehydrogenase. 2-Oxo-3,4-dihydroxybutyric acid is reduced to 2-amino-3,4-dihydroxybutyric acid by amino dehydrogenase/transaminase. Dehydration of 2-amino-3,4-dihydroxybutyric acid produces 2-amino-3,4-dihydroxybutanal which can be further reduced to 3-amino-1,2,4-butanetriol or aminated to 3,4-diamino-1,2-butanediol.

2,3,4-Trihydroxybutyryl-CoA as the product of condensation can be reduced to threitol, 3,4-dihydroxy-2-butanone and 1,2,4-butanetriol. First, 2,3,4-trihydroxybutyryl-CoA is reduced to 2,3,4-trihydroxybutyraldehyde through aldehyde dehydrogenase which can be further reduced to threitol by aldehyde reductase. Dehydration of threitol by diol dehydratase produces 3,4-dihydroxy-2-butanone. Moreover, dehydration of threitol by diol dehydratase produces 3,4-dihydroxybutyraldehyde. The resulting 3,4-dihydroxybutyraldehyde can be fed to the subsequent iteration of C1 elongation or reduced to 1,2,4-butanetriol by aldehyde reductase. 2,3,4-Trihydroxybutyryl-CoA can be used to produce unsaturated acids. 2,3,4-Trihydroxybutyryl-CoA is dehydrated to 3,4-dihydroxycrotonyl-CoA by acyl-CoA dehydratase. 3,4-Dihydroxycrotonyl-CoA is converted to 3,4-dihydroxycrotonic acid through CoA thioesterase. Moreover, 3,4-dihydroxycrotonyl-CoA can be hydrolyzed to 3,3,4-trihydroxybutyryl-CoA by acyl-CoA dehydratase and further oxidized to 3,3,4-trihydroxybutyric acid by acyl-CoA thioesterase. Another pathway for 3,4-dihydroxycrotonyl-CoA is to be converted to 3,4-dihydroxybutyryl-CoA by acyl-CoA dehydrogenase. 3,4-Dihydroxybutyryl-CoA is further reduced to 3,4-dihydroxybutyraldehyde by aldehyde dehydrogenase.

### Example 36: Carboxylic Acid Platform Using Oxalic Acid-Derived Oxalic Semialdehyde as Substrate for Condensation with Formyl-CoA

The purpose of this example is to demonstrate the implementation of the carboxylic acid (CA) platform using oxalic acid (R═OOH) as the CA intermediate with C1 elongation pathways.

Oxalic acid can be activated to oxalyl-CoA through oxalyl-phosphate. First, oxalic acid is phosphorylated to oxalyl-phosphate by kinase and then converted to oxalyl-CoA by phosphotransacylase. The other pathway to activate oxalic acid is oxalate-CoA ligase from Arabidopsis thaliana (Foster et al. Plant Cell. 24(3):1217-1229 (2012)) or CoA transferase from 20 E. coli (Mullins et al. PLoS One. 8(7):e67901 (2013)). Finally, oxalyl-CoA is reduced to glyoxylic acid by aldehyde dehydrogenase.

C1 elongation is initiated by formyl-CoA production through energy and formyl-CoA generation steps. 2-Hydroxyacyl-CoA synthase (HACS) condenses glyoxylic acid and formyl-CoA to produce tartronyl-CoA. Tartronyl-CoA is converted to tartronic acid by acyl-CoA thioesterase.

Tartronic acid can be further oxidized to mesoxalic acid by 2-hydroxyacid dehydrogenase. Mesoxalic acid is reduced to aspartic acid by amino dehydrogenase/transaminase. Dehydration of Aminomalonic acid produces 2-amino-3-oxopropionic acid which can be further reduced to serine or aminated to 3-aminoalanine.

Tartronyl-CoA as the product of condensation can be reduced to glyceric acid, pyruvic acid and 3-hydroxypropionic acid. First, tartronyl-CoA is reduced to 2-hydroxy-3-oxopropionic acid through aldehyde dehydrogenase which can be further reduced to glyceric acid by aldehyde reductase. Dehydration of glyceric acid by diol dehydratase produces pyruvic acid. Moreover, dehydration of glyceric acid by diol dehydratase produces 3-oxopropionic acid. The resulting 3-oxopropionic acid can be fed to the subsequent iteration of C1 elongation or reduced to 3-hydroxypropionic acid by aldehyde reductase.

To implement the C1 elongation pathway for tartronic acid production in vivo, we engineered vectors to independently control expression of various HACS candidates and the acyl-CoA transferase from Clostridium aminobutyricum (CaAbfT), with HACS under control of the IPTG-inducible T7 promoter in pCDFDuet-1 and CaAbfT under control of a cumate-inducible T5 promoter in pETDuet-1. As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD).

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hours) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside and cumate) were added. Plates were incubated for a total of 24 hours post-inoculation.

Cells from the above pre-cultures were then centrifuged (4000 rpm, 22° C.), washed with the above minimal media without any carbon source, and resuspended with 1 mL of above minimal media containing indicated amounts of carbon source. 5 mM glyoxylic acid and 20 mM formate were added at 0 hr and were incubated at 30° C. and 1000 rpm in Digital Microplate Shaker (Fisher Scientific). After incubation at 30° C. for 1 hour, the cells were pelleted by centrifugation and the media was analyzed using HPLC.

The result indicates that RuHACL is the best candidate under the given experimental conditions reaching up to 13 μM/hr per OD600.

### Example 37: Carboxylic Acid Platform Using Malonic Acid-Derived Malonic Semialdehyde as Substrate for Condensation with Formyl-CoA

The purpose of this example is to demonstrate the implementation of the carboxylic acid (CA) platform using malonic acid (R═COOH) as the CA intermediate with C₁ elongation pathways.

Malonic acid can be activated to malonyl-CoA through malonyl-phosphate. First, malonic acid is phosphorylated to malonyl-phosphate by kinase and then converted to malonyl-CoA by phosphotransacylase. The other pathway to activate malonic acid is CoA synthetase or CoA transferase. Finally, malonyl-CoA is reduced to malonic semialdehyde by aldehyde dehydrogenase.

C1 elongation is initiated by formyl-CoA production through energy and formyl-CoA generation steps. 2-Hydroxyacyl-CoA synthase (HACS) condenses malonic semialdehyde and formyl-CoA to produce malyl-CoA. Malyl-CoA is converted to malic acid by acyl-CoA thioesterase.

Malic acid can be further oxidized to 2-oxosuccinic acid (oxalacetic acid) by 2-hydroxyacid dehydrogenase. 2-Oxosuccinic acid is reduced to aspartic acid by amino dehydrogenase/transaminase. Dehydration of aspartic acid produces 3-amino-4-oxobutyric acid which can be further reduced to 3-amino-4-hydroxybutyric acid or aminated to 3,4-diaminobutyric acid.

Malyl-CoA as the product of condensation can be reduced to 3,4-dihydroxybutyric acid, acetoacetic acid and 4-hydroxybutyric acid. First, malyl-CoA is reduced to 3-hydroxy-4-oxobutyric acid through aldehyde dehydrogenase which can be further reduced to 3,4-dihydroxybutyric acid by aldehyde reductase. Dehydration of 3,4-dihydroxybutyric acid by diol dehydratase produces acetoacetic acid. Moreover, dehydration of 3,4-dihydroxybutyric acid by diol dehydratase produces 4-oxobutyric acid. The resulting 4-oxobutyric acid can be fed to the subsequent iteration of C1 elongation or reduced to 4-hydroxybutyric acid by aldehyde reductase.

Malyl-CoA can be used to produce unsaturated acids. Malyl-CoA is dehydrated to fumaryl-CoA by acyl-CoA dehydratase. Fumaryl-CoA is converted to fumaric acid through CoA thioesterase. Moreover, fumaryl-CoA can be hydrolyzed to 2-hydroxysuccinyl-CoA by acyl-CoA dehydratase and further oxidized to 2-hydroxysuccinic acid by acyl-CoA thioesterase. Another pathway for fumaryl-CoA is to be converted to succinyl-CoA by acyl-CoA dehydrogenase. Succinyl-CoA is further reduced to 4-oxobutyric acid by aldehyde dehydrogenase.

### Example 38: Carboxylic Acid Platform Using Isobutyric Acid-Derived Isobutyraldehyde as Substrate for Condensation with Formyl-CoA

The purpose of this example is to demonstrate the implementation of the carboxylic acid (CA) platform using isobutyric acid (R═CH₃CH₃) as the CA intermediate with C1 elongation pathways.

Isobutyric acid can be activated to isobutyryl-CoA through isobutyryl-phosphate. First, isobutyric acid is phosphorylated to isobutyryl-phosphate by kinase and then converted to isobutyryl-CoA by phosphotransacylase. The other pathway to activate isobutyric acid is CoA synthetase or CoA transferase. Finally, isobutyryl-CoA is reduced to isobutyraldehyde by aldehyde dehydrogenase.

C1 elongation is initiated by formyl-CoA production through energy and formyl-CoA generation steps. 2-Hydroxyacyl-CoA synthase (HACS) condenses isobutyraldehyde and formyl-CoA to produce 2-hydroxy-3-methylbutyryl-CoA. 2-Hydroxy-3-methylbutyryl-CoA is converted to 2-hydroxyisovaleric acid by acyl-CoA thioesterase.

2-Hydroxyisovaleric acid can be further oxidized to 2-oxoisovaleric acid by 2-hydroxyacid dehydrogenase. 2-Oxoisovaleric acid is reduced to valine by amino dehydrogenase/transaminase. Dehydration of valine produces 2-amino-3-methylbutanal which can be further reduced to valinol or aminated to 3-methylbutane-1,2-diamine.

2-Hydroxy-3-methylbutyryl-CoA as the product of condensation can be reduced to 3-methylbutane-1,2-diol, 3-methyl-2-butanone and isoamyl alcohol. First, 2-hydroxy-3-methylbutyryl-CoA is reduced to 2-hydroxy-3-methylbutanal through aldehyde dehydrogenase which can be further reduced to 3-methylbutane-1,2-diol by aldehyde reductase. Dehydration of 3-methylbutane-1,2-diol by diol dehydratase produces 3-methyl-2-butanone. Moreover, dehydration of 3-methylbutane-1,2-diol by diol dehydratase produces isovaleraldehyde. The resulting isovaleraldehyde can be fed to the subsequent iteration of C1 elongation or reduced to isoamyl alcohol by aldehyde reductase.

2-Hydroxy-3-methylbutyryl-CoA can be used to produce unsaturated acids. 2-Hydroxy-3-methylbutyryl-CoA is dehydrated to 3-methylcrotonyl-CoA by acyl-CoA dehydratase. 3-Methylcrotonyl-CoA is converted to 3-methylcrotonic acid through CoA thioesterase. Moreover, 3-methylcrotonyl-CoA can be hydrolyzed to 3-hydroxy-3-methylbutyryl-CoA by acyl-CoA dehydratase and further oxidized to 3-hydroxyisovaleric acid by acyl-CoA thioesterase. Another pathway for 3-methylcrotonyl-CoA is to be converted to 3-methylbutyryl-CoA by acyl-CoA dehydrogenase. 3-Methylbutyryl-CoA is further reduced to isovaleraldehyde by aldehyde dehydrogenase.

### Tables 1-10

TABLE 1
List of 2-hydroxyacyl-CoA (HACS) variants (JGI) identified
by selecting representative genes from gene clusters with
sequence similarity using RuHACL as reference enzyme

| JGI# | GenBank Accession Number |
|---|---|
| 1 | XP_012756082.1 |
| 2 | TMK01573.1 |
| 3 | PYM26381.1 |
| 4 | EEG70177.1 |
| 5 | MBH80817.1 |
| 6 | WP_030891887.1 |
| 7 | AGK93615.1 |
| 8 | MAX57815.1 |
| 9 | WP_068916287.1 |
| 10 | WP_062165271.1 |
| 11 | MBB43458.1 |
| 12 | PCJ72347.1 |
| 13 | TMQ19149.1 |
| 14 | MAX11513.1 |
| 15 | HAK63664.1 |
| 16 | MBG92919.1 |
| 17 | PZC46201.1 |
| 18 | MBB84818.1 |
| 19 | OGA51379.1 |
| 20 | PWB41796.1 |
| 21 | MAE93843.1 |
| 22 | OGP60024.1 |
| 23 | OWB57166.1 |
| 24 | KXN72624.1 |
| 25 | PVU86112.1 |
| 26 | ORZ16580.1 |
| 27 | XP_005644825.1 |
| 28 | KZV27770.1 |
| 29 | EJY87672.1 |

TABLE 2
List of 2-hydroxyacyl-CoA (HACS) variants (JGIH)
identified by selecting representative genes from
gene clusters with sequence similarity using AcHACS,
JGI15, JGI19 and JGI20 as reference enzymes

| JGIH# | GenBank Accession Number |
|---|---|
| 1 | HIG47824.1 |
| 2 | TMD03111.1 |
| 3 | MBJ56818.1 |
| 4 | WP_095860310.1 |
| 5 | MBL8483477.1 |
| 6 | WP_058697592.1 |
| 7 | WP_130292058.1 |
| 8 | WP_207956071.1 |
| 9 | WP_132429652.1 |
| 10 | WP_060575023.1 |
| 11 | WP_068796145.1 |
| 12 | OJY48151.1 |
| 13 | WP_062397209.1 |
| 14 | WP_169186431.1 |
| 15 | WP_133828190.1 |
| 16 | MBS0560157.1 |
| 17 | PCJ59575.1 |
| 18 | MXY78649.1 |
| 19 | MBA01399.1 |
| 20 | MXX31676.1 |
| 21 | MXV80929.1 |
| 22 | MBI4083577.1 |
| 23 | MBK6319978.1 |
| 24 | MBI5948182.1 |
| 25 | PFG74273.1 |
| 26 | WP_158065972.1 |
| 27 | MBN9492325.1 |
| 28 | MBK6663287.1 |
| 29 | MBI2766664.1 |
| 30 | HEM18354.1 |
| 31 | GBD22648.1 |
| 32 | MBF6599205.1 |
| 33 | MXW00101.1 |
| 34 | MYA07641.1 |
| 35 | REJ76484.1 |
| 36 | HDY15625.1 |
| 37 | MBW2231087.1 |
| 38 | NRA08835.1 |
| 39 | NQZ98823.1 |
| 40 | MBI3918747.1 |
| 41 | MBI2761137.1 |
| 42 | MBE0608783.1 |
| 43 | MYA54281.1 |
| 44 | NRA01576.1 |
| 45 | MBW2623123.1 |
| 46 | MBI5615765.1 |
| 47 | MSR14309.1 |
| 48 | XP_004342722.2 |
| 49 | MSP42197.1 |
| 50 | TDI61101.1 |
| 51 | MBO0741576.1 |
| 52 | MBO0736096.1 |
| 53 | MBV9828771.1 |
| 54 | MAW55136.1 |
| 55 | MBV38827.1 |
| 56 | TMJ68231.1 |
| 57 | TMJ64557.1 |
| 58 | MBV9815528.1 |
| 59 | MYH41266.1 |
| 60 | MPZ97997.1 |
| 61 | MBT5774752.1 |
| 62 | XP_014714961.1 |
| 63 | TAK78428.1 |
| 64 | TAJ19927.1 |
| 65 | PKN81274.1 |
| 66 | RLT34960.1 |
| 67 | MBT5775398.1 |
| 68 | TMD99851.1 |
| 69 | MSQ12864.1 |
| 70 | MBL0714078.1 |
| 71 | WP_114297888.1 |
| 72 | MAK25262.1 |
| 73 | WP_068138361.1 |
| 74 | RMG94145.1 |
| 75 | MBA4180234.1 |
| 76 | MBM3723043.1 |
| 77 | ABF11225.1 |
| 78 | TAL98798.1 |
| 79 | NNN20496.1 |
| 80 | MBP1761901.1 |
| 81 | PPQ43247.1 |
| 82 | MSQ25793.1 |
| 83 | TMK28344.1 |
| 84 | HIB12002.1 |
| 85 | WP_179589464.1 |
| 86 | MXY42918.1 |
| 87 | WP_184156128.1 |
| 88 | HET53513.1 |
| 89 | TMK22624.1 |
| 90 | MXX66290.1 |
| 91 | GIS94895.1 |
| 92 | MBN1557905.1 |
| 93 | MSV30368.1 |
| 94 | MBN2179295.1 |
| 95 | TDI90456.1 |
| 96 | OGN76415.1 |
| 97 | WP_102074055.1 |
| 98 | PZC47999.1 |
| 99 | HHH88785.1 |
| 100 | OLB93949.1 |
| 101 | PKB76696.1 |
| 102 | HED24197.1 |
| 103 | WP_066960443.1 |
| 104 | WP_169259343.1 |
| 105 | WP_201494572.1 |
| 106 | MBN9621549.1 |
| 107 | OZG26106.1 |
| 108 | WP_016501746.1 |

TABLE 3
List of enzymes for one-carbon compounds
interconversion and formyl-CoA generation

| | | | Uniprot |
|---|---|---|---|
| Reaction | Gene | Organism | accession |
| sMMO | mmoXYBZCD | Methylococcus | P18797 |
| | | capsulatus | |
| pMMO | pmoA1A2B1B2 | Methylococcus | G1UBD1 |
| | | capsulatus | Q607G3 |
| MDH | BmMDH2 | Bacillus | I3E2P9 |
| | | methanolicus | |
| | | MGA3 | |
| | CnMDH2 | Cupriavidus | F8GNE5 |
| | | necator | |
| | BsMDH | Bacillus | P42327 |
| | | stearothermophilus | |
| ACR1 | LmACR | Listeria | Q8Y7U1 |
| | | monocytogenes | |
| | StEutE | Salmonella | P41793 |
| | | typhymurium | |
| | CbAld | Clostridium | Q716S8 |
| | | beijerinckii | |
| | EcMhpF | Escherichia coli | P77580 |
| | PsDmpF | Pseudomonas sp. | Q52060 |
| | | strain CF600 | |
| ALDH | EcFrmA | Escherichia coli | P25437 |
| | PpFdhA | Pseudomonas | P46154 |
| | | putida | |
| PTA-FOK | CcPta-Ack | Clostridium | A0A0J8D6J2 |
| | | cylindrosporum | A0A0J8DB00 |
| | EcPta-Ack | Escherichia coli | P0A9M8 |
| | | | P0A6A3 |
| ACS | EcACS | Escherichia coli | P27550 |
| | StACSstab | Salmonella | Q8ZKF6_PROSS |
| | | typhimurium | |
| | MhACS | Marinithermus | F2NQX2 |
| | | hydrothemalis | |
| | ArACS | Angustibacter sp. | A0A0Q7JEV7 |
| | | Root456 | |
| ACT | CaAbfT | Clostridium | Q9RM86 |
| | | aminobutyricum | |
| | OfFrc | Oxalobacter | O06644 |
| | | formigenes | |
| FDH | PsFdh | Pseudomonas sp. | P33160 |
| | | (strain 101) | |
| | CbFdh | Candida boidinii | O13437 |

TABLE 4
Summary table for different aldehydes that can be utilized by HACS as substrates

| R group | Substrate | Product |
|---|---|---|
| NH₂ | 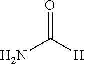 | 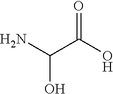 |
| | Formamide | 2-Hydroxyglycine |
| Phenyl | 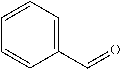 | 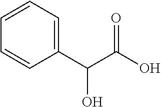 [SMILES: O=C(O)C(O)c1ccccc1] |
| | Benzaldehyde | Mandelic acid |
| H | 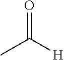 | 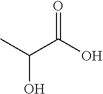 |
| | Acetaldehyde | Lactate |
| CH₃ | 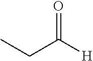 | 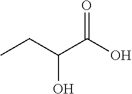 |
| | Propionaldehyde | 2-Hydroxybutrate |
| OH | 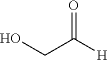 | 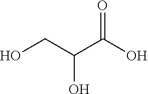 |
| | Glycolaldehyde | Glycerate |
| OOH | 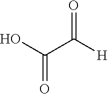 | 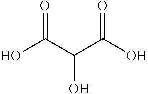 |
| | Glyoxylate | Tartronic acid |
| CH₂OH | 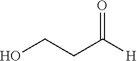 | 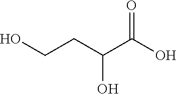 |
| | 3- | 2,4- |
| | Hydroxypropionaldehyde | Dihydroxybutrate |
| COOH | 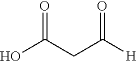 | 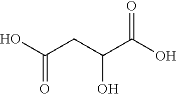 |
| | Malonic semialdehyde | Malic acid |
| CH₂COOH | 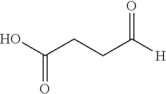 | 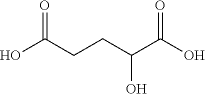 |
| | Succinic semialdehyde | 2-Hydroxyglutaric |
| | | acid |
| CH(CH₃) | 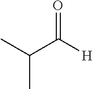 | 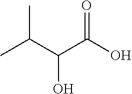 |
| | Isobuteraldehyde | 2-Hydroxyisovaleric |
| | | acid |
| CH(CH₃)₂ | 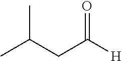 | 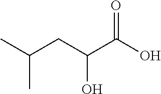 |
| | Isovaleraldehyde | Leucic acid |
| CSCH₃ | 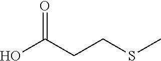 | 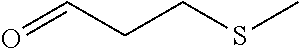 |
| | 3-(methylthio)propanoic | methional |
| | acid | |
| (CH₃)₂CH₂OH | 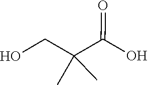 | 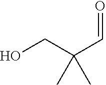 |
| | 3-hydroxy-2,2- | Hydroxypivaldehyde |
| | dimethylpropanoic acid | |

TABLE 5
Activities of HACS variants on different aldehydes for 2-hydroxyacid
production. Numbers present product flux in μM/OD/h.

| | Substrate | | | | | |
|---|---|---|---|---|---|---|
| | | | | | Succinic | |
| | Formaldehyde | Acetaldehyde | Glycolaldehyde | Methional | semialdehyde | Isovaleraldehyde |
| | Product | | | | | |
| | Glycolate | Lactate | Glycerate | Desmeninol | 2-hydroxyglutarate | Leucic acid |
| RuHACL | 44.5 | 78.1 | 7.2 | 20.0 | 15.2 | 12.5 |
| AcHACS | 37.0 | 12.7 | 3.6 | −0.8 | 1.2 | 3.0 |
| JGI1 | 0.0 | 42.3 | 2.1 | 22.7 | 1.2 | 14.0 |
| JGI2 | 0.0 | 6.6 | 5.3 | −2.0 | 4.2 | 8.1 |
| JGI15 | 194.8 | 81.9 | 14.5 | 16.5 | 19.5 | 9.9 |
| JGI19 | 53.9 | 22.8 | 8.1 | −1.2 | 11.5 | 7.0 |
| JGI20 | 206.2 | 81.4 | 23.2 | 22.1 | 49.0 | 12.2 |
| JGI23 | 0.0 | 15.0 | 7.1 | −1.1 | 2.2 | 12.3 |
| JGI24 | 0.0 | 34.0 | 1.6 | 27.5 | 1.1 | 20.6 |
| JGI29 | 0.0 | 16.3 | 5.4 | 12.6 | 1.2 | 7.6 |
| JGIH4 | 102.4 | 42.5 | 21.7 | 17.7 | 1.1 | 2.9 |
| JGIH5 | 175.3 | 41.7 | 29.2 | 28.2 | 1.5 | 10.5 |
| JGIH7 | 128.1 | 29.9 | 24.8 | 18.3 | 1.3 | 5.9 |
| JGIH12 | 146.3 | 36.9 | 21.3 | 25.4 | 1.1 | 7.2 |
| JGIH13 | 93.6 | 31.1 | 18.8 | 14.8 | 1.2 | 6.0 |
| JGIH14 | 66.1 | 22.3 | 9.8 | 13.6 | 1.4 | 5.9 |
| JGIH24 | 127.8 | 74.9 | 15.2 | 22.0 | 40.2 | 14.0 |
| JGIH25 | 247.8 | 48.5 | 21.5 | 19.5 | 31.5 | 12.2 |
| JGIH26 | 211.3 | 47.6 | 21.8 | 20.3 | 24.4 | 10.3 |
| JGIH27 | 47.2 | 35.8 | 11.3 | 11.3 | 18.6 | 7.6 |
| JGIH28 | 181.4 | 61.3 | 21.9 | 15.1 | 41.2 | 11.7 |
| JGIH30 | 161.4 | 32.2 | 16.8 | 21.7 | 21.7 | 8.6 |
| JGIH31 | 92.7 | 27.3 | 9.3 | 21.8 | 14.4 | 7.4 |
| JGIH35 | 101.2 | 19.3 | 7.7 | −1.2 | 3.3 | 5.5 |
| JGIH36 | 115.0 | 20.2 | 16.4 | 15.7 | 22.0 | 7.4 |
| JGIH41 | 75.7 | 40.1 | 3.6 | 15.2 | 10.0 | 8.3 |
| JGIH48 | 133.5 | 132.0 | 17.7 | 33.9 | 13.3 | 17.8 |
| JGIH50 | 126.8 | 42.1 | 8.9 | −1.0 | 9.2 | 8.5 |
| JGIH52 | 0.0 | 23.9 | 4.5 | −1.0 | 8.2 | 8.9 |
| JGIH59 | 0.0 | 50.1 | 13.1 | 19.8 | 29.3 | 12.3 |
| JGIH60 | 68.6 | 61.4 | 19.8 | 15.1 | 29.3 | 13.3 |
| JGIH61 | 108.5 | 93.6 | 13.6 | 31.7 | 35.2 | 18.6 |
| JGIH64 | 193.3 | 82.3 | 20.5 | 26.6 | 31.3 | 13.4 |
| JGIH65 | 349.3 | 120.2 | 37.0 | 42.3 | 34.8 | 19.7 |
| JGIH66 | 84.9 | 37.2 | 4.3 | 18.1 | 13.7 | 10.1 |
| JGIH67 | 100.6 | 58.2 | 13.6 | 24.6 | 32.5 | 13.1 |
| JGIH76 | 61.9 | 46.1 | 22.9 | 18.6 | 1.9 | 8.4 |

TABLE 6

| | | | Uniprot |
|---|---|---|---|
| Reaction | Gene | Organism | accession |
| PTA-FOK | CcPta-Ack | Clostridium | A0A0J8D6J2 |
| | | cylindrosporum | A0A0J8DB00 |
| | EcPta-Ack | Escherichia coli | P0A9M8 |
| | | | P0A6A3 |
| ACS | EcACS | Escherichia coli | P27550 |
| | StACSstab | Salmonella | Q8ZKF6_PROSS |
| | | typhimurium | |
| | MhACS | Marinithermus | F2NQX2 |
| | | hydrothemalis | |
| | ArACS | Angustibacter sp. | A0A0Q7JEV7 |
| | | Root456 | |
| ACT | CaAbfT | Clostridium | Q9RM86 |
| | | aminobutyricum | |
| | OfFrc | Oxalobacter | O06644 |
| | | formigenes | |
| ACR1 | LmACR | Listeria | Q8Y7U1 |
| | | monocytogenes | |
| HACS | ApbHACS | Alphaproteobacteria | A0A3C0TX30 |
| | | bacterium | |
| | DhcHACS | Dehalococcoidia | A0A315XEK8 |
| | | bacterium | |
| | RcbHACS | Rhodocyclaceae | MBL8483477.1 |
| | | bacterium | (GenBank) |
| | CfgHACS | Chloroflexi | A0A2A9HFZ7 |
| | | bacterium (strain | |
| | | G233) | |
| | CoHACS | Capsaspora | A0A0D2WIE0 |
| | | owczarzaki (strain | |
| | | ATCC 30864) | |
| | CfhHACS | Chloroflexi | A0A2N2LHX4 |
| | | bacterium HGW- | |
| | | Chloroflexi-9 | |
| ACR2 | LmACR | Listeria | Q8Y7U1 |
| | | monocytogenes | |
| | StPduP | Salmonella | Q9XDN1 |
| | | typhimurium | |
| ALD | EcAldA | Escherichia coli | P25553 |
| ADH1 | EcfucO | Escherichia coli | P0A9S1 |
| DDR | KoPddABC | Klebsiella oxytoca | Q59470 |
| | | | Q59471 |
| | | | Q59472 |
| ADH2 | EcAdhE | Escherichia coli | P0A9Q7 |
| PTA2- | CcPta-Ack | Clostridium | A0A0J8D6J2 |
| CAK | | cylindrosporum | A0A0J8DB00 |
| | EcPta-Ack | Escherichia coli | P0A9M8 |
| | | | P0A6A3 |
| TES | EcYciA | Escherichia coli | P0A8Z0 |
| HADH | EcGlcD | Escherichia coli | P0AEP9 |
| AADH | MtAld | Mycobacterium | P9WQB1 |
| | | tuberculosis | |
| | BsAld | Bacillus subtilis | Q08352 |
| TA1 | ALATI | Homo Sapiens | P24298 |
| ALDH2 | aldH1 | Aquifex aeolicus | O66573 |
| | dhaS | Anoxybacillus | B7GJB2 |
| | | flavithermus | |
| TA2 | EcSerC | Escherichia coli | P23721 |
| | GOT1 | Sus scrofa | P00503 |
| ADH3 | EcYdfG | Escherichia coli | P39831 |
| | EcGldA | Escherichia coli | P0A9S5 |

TABLE 7

| | | | Uniprot |
|---|---|---|---|
| Reaction | Gene | Organism | accession |
| FMD | MmFmdA | Methylophilus | Q50228 |
| | | methylotrophus | |
| | | (Bacterium W3A1) | |
| HACS | ApbHACS | Alphaproteobacteria | A0A3C0TX30 |
| | | bacterium | |
| | DhcHACS | Dehalococcoidia | A0A315XEK8 |
| | | bacterium | |
| | RcbHACS | Rhodocyclaceae | MBL8483477.1 |
| | | bacterium | (GenBank) |
| | CfgHACS | Chloroflexi | A0A2A9HFZ7 |
| | | bacterium (strain | |
| | | G233) | |
| | CoHACS | Capsaspora | A0A0D2W1E0 |
| | | owczarzaki (strain | |
| | | ATCC 30864) | |
| | CfhHACS | Chloroflexi | A0A2N2LHX4 |
| | | bacterium HGW- | |
| | | Chloroflexi-9 | |
| ACR2 | LmACR | Listeria | Q8Y7U1 |
| | | monocytogenes | |
| ALD | EcAldA | Escherichia coli | P25553 |
| ADH1 | EcfucO | Escherichia coli | P0A9S1 |
| DDR | KoPddABC | Klebsiella oxytoca | Q59470 |
| | | | Q59471 |
| | | | Q59472 |
| ADH2 | EcAdhE | Escherichia coli | P0A9Q7 |
| PTA2- | CcPta-Ack | Clostridium | A0A0J8D6J2 |
| CAK | | cylindrosporum | A0A0J8DB00 |
| HADH | EcPta-Ack | Escherichia coli | P0A9M8 |
| | | | P0A6A3 |
| TES | EcYciA | Escherichia coli | P0A8Z0 |
| HADH | EcGlcD | Escherichia coli | P0AEP9 |
| AADH | MtAld | Mycobacterium | P9WQB1 |
| | | tuberculosis | |
| | BsAld | Bacillus subtilis | Q08352 |
| TA1 | ALAT1 | Homo Sapiens | P24298 |
| ALDH2 | aldH1 | Aquifex aeolicus | O66573 |
| | dhaS | Anoxybacillus | B7GJB2 |
| | | flavithermus | |
| TA2 | EcSerC | Escherichia coli | P23721 |
| | GOT1 | Sus scrofa | P00503 |
| ADH3 | EcYdfG | Escherichia coli | P39831 |
| | EcGldA | Escherichia coli | P0A9S5 |

TABLE 8
R group for C₁ carboxylic acids and corresponding aldehydes for CA platform

| | Carboxylic acid | Aldehyde |
|---|---|---|
| R group | Compound and Structure | Compound and Structure |
| H | 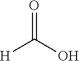 | 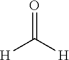 |
| | Formic acid | Formaldehyde |
| NH₂ | 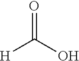 | 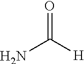 |
| | Formic acid | Formamide |
| Phenyl | 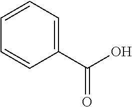 | 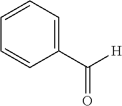 |
| | Benzoic acid | Benzaldehyde |

TABLE 9
R group for C₂+ carboxylic acids and corresponding aldehydes for CA platform

| | Carboxylic acid | Aldehyde |
|---|---|---|
| R group | Compound and Structure | Compound and Structure |
| H | 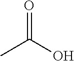 | 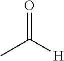 |
| | Acetic acid | Acetaldehyde |
| CH₃ | 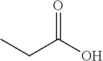 | 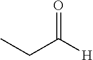 |
| | Propionic acid | Propionaldehyde |
| CH₂CH₃ | 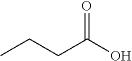 | 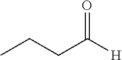 |
| | Butyric acid | Butyraldehyde |
| (CH₂)₂CH₃ | 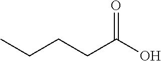 | 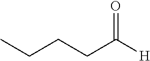 |
| | Pentanoic acid | Valeraldehyde |
| (CH₂)₃CH₃ | 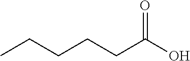 | 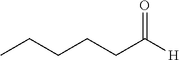 |
| | Hexanoic acid | Hexanal |
| (CH₂)₄CH₃ | 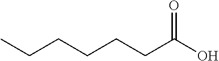 | 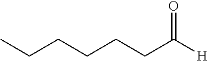 |
| | Heptanoic acid | Heptanal |
| (CH₂)₅CH₃ | 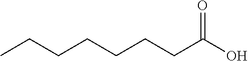 | 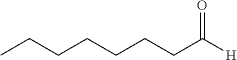 |
| | Octanoic acid | Octanal |
| (CH₂)₆CH₃ | 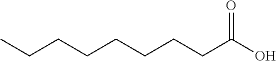 | 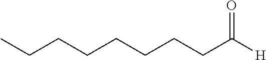 |
| | Nonanoic acid | Nonanal |
| OH | 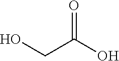 | 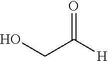 |
| | Glycolic acid | Glycolaldehyde |
| CH₃OH | 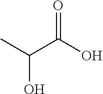 | 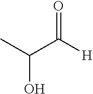 |
| | Lactic acid | Lactaldehyde |
| OHCH₂OH | 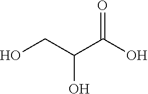 | 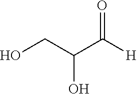 |
| | Glyceric acid | Glyceraldehyde |
| OOH | 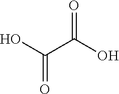 | 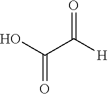 |
| | Oxalic acid | Glyoxylic acid |
| CH₂OH | 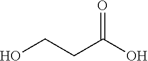 | 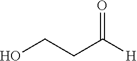 |
| | 3-Hydroxypropionic acid | 3-Hydroxypropionaldehyde |
| COOH | 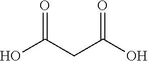 | 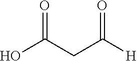 |
| | Malonic acid | Malonic semialdehyde |
| CH₂COOH | 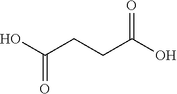 | 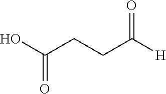 |
| | Succinic acid | Succinic semialdehyde |
| (CH₃)₂ | 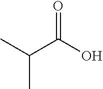 | 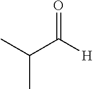 |
| | Isobutyric acid | Isobutyraldehyde |
| CH(CH₃)₂ | 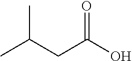 | 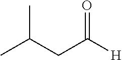 |
| | Isovaleric acid | Isovaleraldehyde |
| Phenyl | 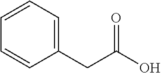 | 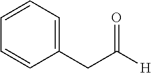 |
| | Phenylacetic acid | Phenylacetaldehyde |

TABLE 10
R₁ and R₂ groups for carboxylic acids and corresponding ketones for CA platform

| | R₂ | Carboxylic acid | Ketone |
|---|---|---|---|
| R₁ group | group | Compound and Structure | Compound and Structure |
| H | CH₃ | 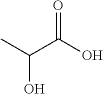 | 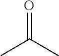 |
| | | Lactic acid | Acetone |
| CH₃ | CH₃ | 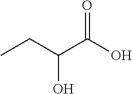 | 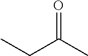 |
| | | 2-Hydroxybutyric acid | 2-Butanone |
| CH₂CH₃ | CH₃ | 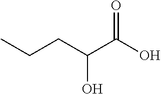 | 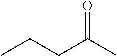 |
| | | 2-Hydroxypentanoic acid | 2-Pentanone |
| (CH₂)₃CH₃ | CH₃ | 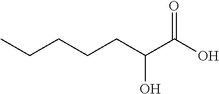 | 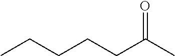 |
| | | 2-Hydroxyheptanoic acid | 2-Heptanone |
| OH | CH₃ | 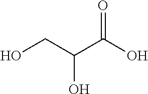 | 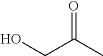 |
| | | 2,3-Dihydroxypropionic acid | Hydroxyacetone |
| (CH₃)₂ | CH₃ | 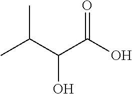 | 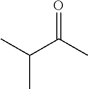 |
| | | 2-Hydroxyisovaleric acid | 3-Methyl-2-butanone |
| O | CH₃ | 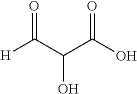 | 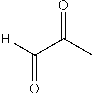 |
| | | Tartronic semialdehyde | Methylglyoxal |
| COCH₃ | CH₃ | 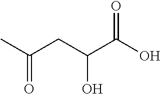 | 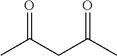 |
| | | 2-Hydroxy-4-oxopentanoic acid | Acetylacetone |

SEQ ID NOs: 1 and 2
RuHACL
(SEQ ID NO: 1)
MSEVDGATLIARSLKQQGIDHLFGVVGFPITAIAAAAQKEGVAYLGMRNEQSAAYAAA
AYGYLTGRPGAAVVVTGPGVVHGLSGLANAQQNCWPMILIGGASETYRGGMGAFQEE
RQVLIASPFCKFAHGIESVARIPFYVEMATRNAIYGRPGATYLDMPDDIIRGTCETDKIAQ
AERVPEAPRSVAPAENVEAALDLLEKAQRPLVLLGKGMAWSRGEDEVRAFIERTQVPF
VRSPMGKGVMPDDHPLSASAARTLALQQADVIFLMGARLNWIFHFGLPPRYAKDVKVI
QLDIAPEEIGHNKPTEVALVGDGKAIMAQLNKALVNRQWFHPKDTPWRQALTKKAAE
NVATIKPQVDDDQGPAGYYRALRDVAAWMPKNAILSAEGANTMDIGLTQLASSNARS
VLNAGTYGTMGVGLGQAIAAAVSDPSRPVIHLSGDSAIGFSGMEMETLVRYNLPVKIVV
LNNGGIGPGMPEIPENPMFNLKPNALIYGARYDKVMEAFGGKGIFVKEPKDIRKALDEA
MAFKGPALVNVVLSQGSTRKAQQFAWHS*
MeOXC4
(SEQ ID NO: 2)
MTVQAQNIDAITAGAMPHEEPELTDGFHLVIDALKLNGIETIYNVPGIPITDLGRLAQAEG
LRVISFRHEQNAGNAAAIAGFLTKKPGICLTVSAPGFLNGLTALANATTNCFPMILISGSS
EREIVDLQQGDYEEMDQLAIAKPLCKAAFRVLHAADIGIGVARAIRAAVSGRPGGVYLD
LPAKLFSQVIDADLGARSLVKVIDAAPAQLPAPAAIARALDVLKSAERPLIILGKGAAYA
QADEAVRALVEESGIPYVPMSMAKGLLPDTHPLSAGAARSTALKDSDVVLLVGARLNW
LLSHGKGKTWGEPGSKRFIQIDIEPREMDSNVEIVAPVVGDIGSCVEALLDGIRKDWKGA
PSNWLETLRGKREANIAKMAPKLMKNSSPMCFHSALGALRTVIKERPDAILVNEGANTL
DLARGIIDMYQPRKRLDVGTWGVMGIGMGFAVAAAVETGKPVLAVEGDSAFGFSGME
VETICRYELPVCIVIFNNNGIARGTDTDPTGRDPGTTVFVKNSRYDKMMEAFGGVGVNV
TTPDELKRAVDEAMNSGKPTLINAEIDPAAGSEAGNIGSLNPQSTLKKK*

Unless otherwise indicated, all numbers expressing reaction conditions, quantities of ingredients, and so forth, as used in this specification and the claims are to be understood as being modified in all instances by the term “about.” While this invention has been particularly shown and described with references to preferred embodiments thereof, it will be understood by those skilled in the art that various changes in form and details may be made therein without departing from the scope of the invention encompassed by the appended claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. The relevant teachings of all patents, published applications and references cited herein are incorporated by reference in their entirety.

1. A genetically modified microorganism that produces a branched product from a ketone and formyl-CoA, the microorganism comprising:
a. at least one heterologous DNA molecule encoding an enzyme that catalyzes the condensation of the ketone with the formyl-CoA to produce a branched 2-hydroxyacyl-CoA that is one carbon longer than the ketone; and
b. one or more heterologous DNA molecules encoding one or more enzymes that catalyze the conversion of the 2-hydroxyacyl-CoA to the product, wherein the product is selected from the group consisting of a 2-hydroxyacid, an ab-unsaturated acid, a 1,2-diol, an alcohol, and a 3-hydroxyacid.

2. The microorganism of claim 1, wherein the enzyme that catalyzes the condensation of the ketone with the formyl-CoA is a 2-hydroxyacyl-CoA synthase (HACS).

3. The microorganism of claim 1, wherein the ketone has the formula (I): 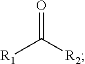
wherein:
R₁ and R₂ are each independently selected from the group consisting of C₁-C₁₂ alkyl, C₂-C₁₂ alkenyl, C₃-C₁₅ cycloalkenyl, aryl, heteroaryl, heterocyclyl, C(O)OR_{b}, C(O)R_{b}, SR_{b}, and N(R_{b})₂; wherein the C₁-C₁₂ alkyl, the C₂-C₁₂ alkenyl, the C₃-C₁₅ cycloalkenyl, the aryl, the heteroaryl, or the heterocyclyl are each optionally substituted by one or more Rₐ;
wherein when one of R₁ and R₂ is C(O)OR_{b} or C(O)R_{b}, the other of R₁ and R₂ is not C(O)OR_{b} or C(O)R_{b};
alternatively, R₁ and R₂ are taken together to form a C₃ to C₇ cycloalkyl, a C₃ to C₇ cycloalkenyl, or a 3- to 7-membered heterocyclyl, wherein the cycloalkyl, cycloalkenyl, or heterocyclyl are each optionally substituted by one or more Rₐ;
each Rₐ is independently selected from the group consisting of hydrogen, optionally substituted C₁-C₁₂ alkyl, optionally substituted C₂-C₁₂ alkenyl, optionally substituted C₂-C₁₂ alkynyl, optionally substituted C₃-C₁₅ cycloalkyl, optionally substituted C₃-C₁₅ cycloalkenyl, optionally substituted aryl, optionally substituted heteroaryl, optionally substituted heterocyclyl, C(O)R_{b}, C(O)OR_{b}, OR_{b}, N(R_{b})₂, halo, and oxo; and
each R_{b} is independently hydrogen or a C₁-C₆ alkyl.

4. The microorganism of claim 3, wherein R₁ and R₂ are different.

5. The microorganism of claim 3, wherein R₁ and R₂ are the same.

6. The microorganism of claim 3, wherein R₂ is a C₁-C₆ alkyl.

7. The microorganism of claim 6, wherein R₂ is methyl or ethyl.

8. The microorganism of claim 6, wherein R₁ is a C₁-C₆ alkyl.

9. The microorganism of claim 6, wherein R₁ and R₂ are each independently C₁-C₆ alkyl substituted by one or more Rₐ wherein the Rₐ is selected from the group consisting of hydrogen, OH and C(O)OH.

10. The microorganism of claim 3, wherein R₁ and R₂ are taken together to form a 3- to 7-membered heterocyclyl, wherein the heterocyclyl comprises a heteroatom selected from oxygen and nitrogen, and wherein the heterocyclyl is optionally substituted by one or more Rₐ.

11. The microorganism of claim 3, wherein the ketone is selected from the group consisting of those in the Table below:
TABLE B 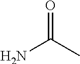
Acetamide 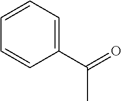
Acetophenone 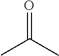
Acetone 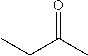
Butanone 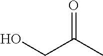
Hydroxyacetone 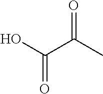
Pyruvic acid 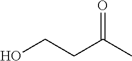
4-hydroxybutan-2-one 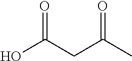
Acetoacetic acid 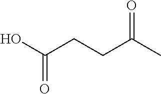
Levulinic acid 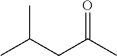
4-methylpentan-2-one 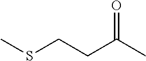
4-Methylthio-2-butanone 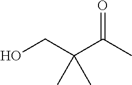
4-hydroxy-3,3-dimethylbutan-2-one 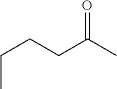
hexan-2-one 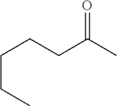
heptan-2-one 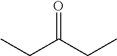
pentan-3-one 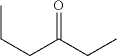
hexan-3-one 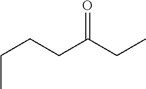
heptan-3-one 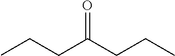
heptan-4-one 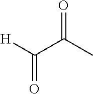
2-oxopropanal 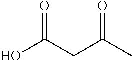
3-oxobutanoic acid
acetoacetate 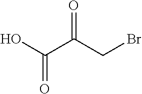
3-bromo-2-oxopropanoic acid 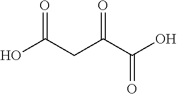
2-oxosuccinic acid
Oxaloacetate 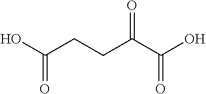
2-oxopentanedioic acid
a-Ketoglutaric acid 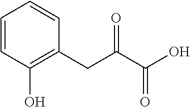 [SMILES: O=C(O)C(=O)Cc1ccccc1O]
3-(2-hydroxyphenyl)-2-oxopropanoic acid 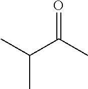
3-methylbutan-2-one 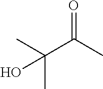
3-hydroxy-3-methylbutan-2-one 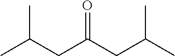
2,6-dimethylheptan-4-one
Diisobutyl ketone 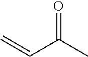
but-3-en-2-one 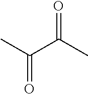
biacetyl 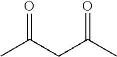
Acetylacetone 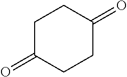 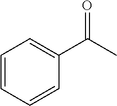
acetophenone 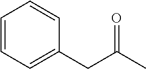
1-phenylpropan-2-one 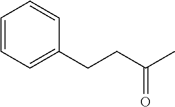
4-phenylbutan-2-one 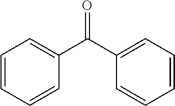 [SMILES: O=C(c1ccccc1)c1ccccc1]
benzophenone 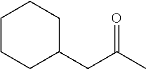
1-cyclohexylpropan-2-one 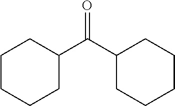 [SMILES: O=C(C1CCCCC1)C1CCCCC1]
Dicyclohexyl ketone 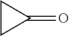
cyclopropanone 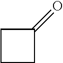
cyclobutanone 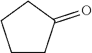
cyclopentanone 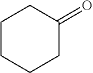
cyclohexanone 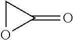
oxiran-2-one 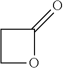
oxetan-2-one 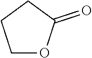
dihydrofuran-2(3H)-one 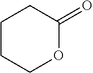
tetrahydro-2H-pyran-2-one 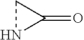
aziridin-2-one 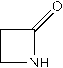
azetidin-2-one 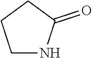
pyrrolidin-2-one 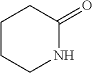
piperidin-2-one 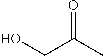
hydroxyacetone 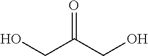
dihydroxyacetone 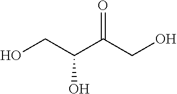
d-erythrulose 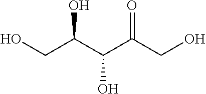
d-ribulose 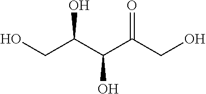
d-xylulose 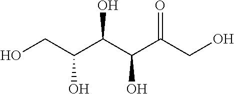 [SMILES: O=C(CO)[C@@H](O)[C@H](O)[C@H](O)CO]
d-fructose 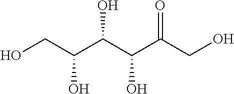 [SMILES: O=C(CO)[C@H](O)[C@@H](O)[C@H](O)CO]
d-sorbose 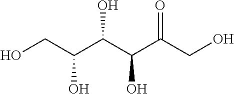 [SMILES: O=C(CO)[C@@H](O)[C@@H](O)[C@H](O)CO]
d-tagatose 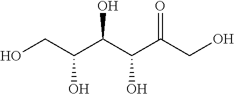 [SMILES: O=C(CO)[C@H](O)[C@H](O)[C@H](O)CO]
d-psicose

12. The microorganism of claim 2, wherein the HACS that catalyzes the condensation of the ketone with the formyl-CoA is selected from those in the Table below:
HACS
AcHACS
ApbHACS
AtsHACS
ApbHACS
BtbHACS
DhcHACS
OxtHACS
RhsHACS
RcbHACS
AspHACS
CfbHACS
CoHACS
CfhHACS
PspHACS
PdsHACS
RuHACS/RuHACL
MeOXC4

13. The microorganism of claim 1, wherein the product is a 2-hydroxyacid and wherein the one or more enzyme that catalyzes the conversion of the 2-hydroxyacyl-CoA to the product comprises:
i. a thioesterase (TES),
ii. an acyl-CoA transferase (ACT), or
iii. a phosphotransacylase (PTA) and a carboxylate kinase (CAK);
or wherein the one or more enzymes that catalyzes the conversion of the 2-hydroxyacyl-CoA to the product comprises:
i. an acyl-CoA reductase for catalyzing the conversion of the said 2-hydroxyacyl-CoA to a 2-hydroxyaldehyde; and
ii. an aldehyde dehydrogenase (ALD) enzyme for converting the 2-hydroxyaldehyde to the 2-hydroxyacid.

14. (canceled)

15. The microorganism of claim 1, wherein the product is a 1,2-diol and wherein the one or more enzymes that catalyze the conversion of the 2-hydroxyacyl-CoA to the product comprises:
i. an acyl-CoA reductase for catalyzing the conversion of the said 2-hydroxyacyl-CoA to a 2-hydroxyaldehyde; and
ii. an alcohol dehydrogenase for catalyzing the conversion of the 2-hydroxyaldehyde to the 1,2-diol.

16. The microorganism of claim 1, wherein the product is an ab-unsaturated acid and wherein the one or more enzymes that catalyze the conversion of the 2-hydroxyacyl-CoA to the product comprises:
i. an acyl-CoA dehydratase (HACD) for catalyzing the dehydration of the 2-hydroxyacyl-CoA to a 2-enoyl-CoA; and
ii. an acyl-CoA transferase, a thioesterase (TES), or a phosphotransacylase (PTA) and a carboxylate kinase (CAK) for catalyzing the conversion of the 2-enoyl-CoA to the ab-unsaturated acid.

17. The microorganism of claim 1, wherein the product is a 3-hydroxyacid and wherein the one or more enzymes that catalyze the conversion of the 2-hydroxyacyl-CoA to the product comprises:
i. an acyl-CoA dehydratase (HACD) for catalyzing the dehydration of the 2-hydroxyacyl-CoA to a 2-enoyl-CoA;
ii. an enoyl-CoA hydratase (ECH) for catalyzing the conversion of the 2-enoyl-CoA to a 3-hydroxy-acyl-CoA; and
iii. an acyl-CoA transferase, a thioesterase, or a phosphotransacylase (PTA) and a carboxylate kinase (CAK) for catalyzing the conversion of the 3-hydroxy-acyl-CoA to the 3-hydroxyacid.

18. The microorganism of claim 1, wherein the product is an alcohol and wherein the one or more enzymes that catalyze the conversion of the 2-hydroxyacyl-CoA to the product comprises:
i. an acyl-CoA reductase for catalyzing the conversion of said 2-hydroxyacyl-CoA to a 2-hydroxyaldehyde;
ii. an alcohol dehydrogenase for catalyzing the conversion of said 2-hydroxyaldehyde to the 1,2-diol;
iii. a diol dehydratase for catalyzing the conversion of the 1,2-diol to an aldehyde; and
iv. an alcohol dehydrogenase for catalyzing the conversion of the aldehyde to the alcohol.

19. The microorganism of claim 1, wherein the product is an alcohol and wherein the one or more enzymes that catalyze the conversion of the 2-hydroxyacyl-CoA to the product comprises:
i. an acyl-CoA dehydratase (HACD) for catalyzing the dehydration of the 2-hydroxyacyl-CoA to a 2-enoyl-CoA;
ii. a trans-2-enoyl-CoA reductase (TER) for catalyzing the conversion of the 2-enoyl-CoA to the acyl-CoA;
iii. an acyl-CoA reductase for catalyzing the conversion of for the acyl-CoA to an aldehyde; and
iv. an alcohol dehydrogenase for catalyzing the conversion of the aldehyde to the alcohol.

20. The microorganism of claim 1, further comprising one or more enzymes for preparing the formyl-CoA from a 1-carbon substrate; optionally wherein:
a. the 1-carbon substate is methanol and the one or more enzymes comprises methanol dehydrogenase for catalyzing the conversion of methanol to formaldehyde, and an acyl-CoA reductase for catalyzing the conversion of the formaldehyde to the formyl-CoA;
b. the 1-carbon substate is formaldehyde and the one or more enzymes comprises an acyl-CoA reductase for catalyzing the conversion of the formaldehyde to the formyl-CoA; or
c. the 1-carbon substrate is formate and the one or more enzymes comprises an acyl-CoA synthetase, an acyl-CoA transferase, or a carboxylate kinase and a phosphotransacylase for catalyzing the conversion of the formate to the formyl-CoA.

21. The microorganism of claim 1, further comprising one or more enzymes for preparing the ketone from a carboxylic acid; wherein the microorganism comprises:
a. one or more enzymes that catalyze the conversion of the carboxylic acid into the corresponding acyl-CoA; and
b. one or more enzymes that catalyze the conversion of the acyl-CoA of the preceding step into the ketone.

22. (canceled)

23. (canceled)

24. The microorganism of claim 15, wherein the ketone is acetone and the 1,2-diol is 2-methylpropane-1,2-diol.

25. The microorganism of claim 16, wherein the ketone is acetone and the αβ-unsaturated acid is methacrylic acid.

26. The microorganism of claim 17, wherein the ketone is acetone and the 3-hydroxyacid is 3-hydroxyisobutyric acid.

27. The microorganism of claim 18, wherein the ketone is acetone and the alcohol is isobutanol.

28. The microorganism of claim 15, wherein the ketone is butan-2-one and the 1,2-diol is 2-methylbutane-1,2-diol.

29. (canceled)

30. (canceled)

31. (canceled)

32. The microorganism of claim 15, wherein the ketone is pentan-2-one and the 1,2-diol is 2-methylbutane-1,2-diol.

33. (canceled)

34. (canceled)

35. (canceled)

36. The microorganism of claim 15, wherein the ketone is heptan-2-one and the 1,2-diol is 2-methylheptane-1,2-diol.

37. (canceled)

38. (canceled)

39. (canceled)

40. The microorganism of claim 15, wherein the ketone is hydroxyacetone and the 1,2-diol is 2-methylpropane-1,2,3-triol.

41. (canceled)

42. (canceled)

43. The microorganism of claim 15, wherein the ketone is 3-methyl-2-butanone and the 1,2-diol is 2,3-dimethylbutane-1,2-diol.

44. (canceled)

45. (canceled)

46. (canceled)

47. The microorganism of claim 15, wherein the ketone is methylglyoxal and the 1,2-diol is 2-methylpropane-1,2,3-triol.

48. (canceled)

49. The microorganism of claim 15, wherein the ketone is pentane-2,4-dione and the 1,2-diol is 4,5-dihydroxy-4-methylpentan-2-one.

50. (canceled)

51. (canceled)

52. (canceled)

53. A method for producing a branched product from a ketone and formyl-CoA, the method comprising growing the microorganism of any one the preceding claims in the presence of a ketone or a carboxylic acid precursor thereof and in the presence of the formyl-CoA or a 1-carbon precursor thereof; the method comprising the steps of:
a. condensing the ketone with the formyl-CoA to produce the branched 2-hydroxyacyl-CoA; and
b. converting the 2-hydroxyacyl-CoA to the product;
wherein when the microorganism is cultured in the presence of the carboxylic acid precursor, the microorganism expresses one more enzymes that catalyze the conversion of the carboxylic acid precursor to the ketone; and
wherein when the microorganism is cultured in the presence of the 1-carbon precursor, the microorganism expresses one or more enzymes that catalyze the conversion of the 1-carbon precursor to the formyl-CoA.

54. The method of claim 53, further comprising isolating the product from the culture medium.

55. A method for producing a branched product from a ketone and formyl-CoA, the method comprising the steps of:
a. growing a genetically modified microorganism in a culture comprising a ketone or a carboxylic acid precursor thereof and comprising formyl-CoA or a 1-carbon precursor thereof;
wherein the microorganism expresses an enzyme that catalyzes the condensation of the ketone with the formyl-CoA to produce a branched 2-hydroxyacyl-CoA that is one carbon longer than the ketone; and
further wherein the microorganism expresses one or more enzymes that catalyze the conversion of the 2-hydroxyacyl-CoA to the product;
b. condensing the ketone with the formyl-CoA to produce the branched 2-hydroxyacyl-CoA; and
c. converting the 2-hydroxyacyl-CoA to the product;
wherein when the culture comprises the carboxylic acid precursor, the microorganism expresses one more enzymes that catalyze the conversion of the carboxylic acid precursor to the ketone; and
wherein when the culture comprises the 1-carbon precursor, the microorganism expresses one or more enzymes that catalyze the conversion of the 1-carbon precursor to the formyl-CoA; and
wherein the product is selected from the group consisting of product is selected from the group consisting of a 2-hydroxyacid, an αβ-unsaturated acid, a 1,2-diol, an alcohol, and a 3-hydroxyacid.

56-107. (canceled)


SEQUENCE LISTING (2 sequences)

SEQ ID NO: 1 - protein, 552 aa, synthetic construct
  source 1..552: note = Rhodospirillales bacterium URHD0017 engineered 2-hydroxyphytanoyl-CoA lyase
MSEVDGATLIARSLKQQGIDHLFGVVGFPITAIAAAAQKEGVAYLGMRNEQSAAYAAAAYGYLTGRPGAAVVVTGPGVVHGLSGLANAQQNCWPMILIGGASETYRGGMGAFQEERQVLIASPFCKFAHGIESVARIPFYVEMATRNAIYGRPGATYLDMPDDIIRGTCETDKIAQAERVPEAPRSVAPAENVEAALDLLEKAQRPLVLLGKGMAWSRGEDEVRAFIERTQVPFVRSPMGKGVMPDDHPLSASAARTLALQQADVIFLMGARLNWIFHFGLPPRYAKDVKVIQLDIAPEEIGHNKPTEVALVGDGKAIMAQLNKALVNRQWFHPKDTPWRQALTKKAAENVATIKPQVDDDQGPAGYYRALRDVAAWMPKNAILSAEGANTMDIGLTQLASSNARSVLNAGTYGTMGVGLGQAIAAAVSDPSRPVIHLSGDSAIGFSGMEMETLVRYNLPVKIVVLNNGGIGPGMPEIPENPMFNLKPNALIYGARYDKVMEAFGGKGIFVKEPKDIRKALDEAMAFKGPALVNVVLSQGSTRKAQQFAWHS

SEQ ID NO: 2 - protein, 583 aa, synthetic construct
  source 1..583: note = Methylorubrum extorquens engineered oxalyl CoA decarboxylase
MTVQAQNIDAITAGAMPHEEPELTDGFHLVIDALKLNGIETIYNVPGIPITDLGRLAQAEGLRVISFRHEQNAGNAAAIAGFLTKKPGICLTVSAPGFLNGLTALANATTNCFPMILISGSSEREIVDLQQGDYEEMDQLAIAKPLCKAAFRVLHAADIGIGVARAIRAAVSGRPGGVYLDLPAKLFSQVIDADLGARSLVKVIDAAPAQLPAPAAIARALDVLKSAERPLIILGKGAAYAQADEAVRALVEESGIPYVPMSMAKGLLPDTHPLSAGAARSTALKDSDVVLLVGARLNWLLSHGKGKTWGEPGSKRFIQIDIEPREMDSNVEIVAPVVGDIGSCVEALLDGIRKDWKGAPSNWLETLRGKREANIAKMAPKLMKNSSPMCFHSALGALRTVIKERPDAILVNEGANTLDLARGIIDMYQPRKRLDVGTWGVMGIGMGFAVAAAVETGKPVLAVEGDSAFGFSGMEVETICRYELPVCIVIFNNNGIARGTDTDPTGRDPGTTVFVKNSRYDKMMEAFGGVGVNVTTPDELKRAVDEAMNSGKPTLINAEIDPAAGSEAGNIGSLNPQSTLKKK